(19) United States
(12) Patent Application Publication
(10) Pub. No.: US 2011/0263642 A1
(43) Pub. Date: Oct. 27, 2011

(54) METHODS FOR TREATMENT OF MULTIPLE SCLEROSIS

(75) Inventors: Stephen J. Klaus, San Francisco, CA (US); Thomas B. Neff, Atherton, CA (US); Gail Walkinshaw, Mountain View, CA (US)

(73) Assignee: FibroGen, Inc., San Francisco, CA (US)

(21) Appl. No.: 12/737,917

(22) PCT Filed: Aug. 26, 2009
(86) PCT No.: PCT/US09/04880

§ 371 Date: Jul. 7, 2011

Related U.S. Application Data
(60) Provisional application No. 61/190,243, filed on Aug. 26, 2008.

(51) Int. Cl. A61K 31/47; A61P 37/06

(52) U.S. Cl. 514/309

(57) ABSTRACT

The present invention relates to methods and compounds useful for treating multiple sclerosis.

This application claims the benefit of U.S. Provisional Application Ser. No. 61/190,243, filed on 26 Aug. 2008, incorporated by reference herein in its entirety.

### FIELD OF THE INVENTION

The present invention relates to methods and compounds useful for treating multiple sclerosis.

### BACKGROUND

Multiple sclerosis (MS) is a chronic degenerative disease affecting the central nervous system, characterized by demyelination of nerve axons. MS may cause numerous physical and mental symptoms, and often progresses to both physical and cognitive disability. Disease onset usually occurs in young adults (20-40 yrs), is more common in women, and affects an estimated 350,000 people in the United States and more than 1 million people around the world.

The disease course of MS is varied and may lie dormant or progress steadily over time. Several subtypes of MS have been described based on patterns of progression. (See Lublin and Reingold (1996) Neurology 46:907-911.) Relapsing-remitting MS (RRMS) occurs in approximately 80-85% of MS patients and is characterized by unpredictable attacks (i.e., relapses) followed by periods of months to years of relative quiet remission with no new signs of disease activity. Deficits suffered during the attacks associated with RRMS may either resolve or may be permanent. Secondary progressive MS (SPMS), which affects approximately 50-80% of patients with RRMS, is characterized by gradual progression of disability (e.g., neurologic decline) with or without superimposed relapses. Primary progressive MS (PPMS), which tends to affect people who are older at time of disease onset, is characterized by a gradual and continuous progression of disability from disease onset without superimposed relapses. Progressive relapsing MS (PRMS) is characterized by gradual progression of disability from disease onset and later accompanied by one or more relapses. PRMS affects approximately 5% of MS patients. It is not fully understood whether these different disease progressions are based on the same or different pathophysiological processes.

MS has no cure. Several current therapies have proven beneficial in restoring function after an attack (relapse), preventing or reducing the degree or frequency of new attacks (relapses), or preventing or reducing the extent of disability. However, many current MS therapies have been associated with adverse effects or are poorly tolerated.

Accordingly, there exists a need for therapies which are effective at treating MS and at alleviating or reducing the symptoms of MS. The present invention meets these needs by providing methods and compounds for treating MS.

### SUMMARY OF THE INVENTION

The present invention relates to methods and compounds useful for treating multiple sclerosis. In one embodiment, the present invention provides a method for treating MS in a subject, the method comprising administering to the subject an effective amount of an agent that stabilizes HIF-1α, thereby treating MS. In one embodiment, the agent that stabilizes HIF-1α is a compound that inhibits the activity of a HIF prolyl hydroxylase enzyme, and the present invention provides methods for treating MS in a subject, wherein the method comprises administering to the subject an effective amount of a compound that inhibits the activity of a HIF prolyl hydroxylase enzyme, thereby treating MS.

The present methods for treatment of MS in a subject are applicable to any subtype of MS or pattern of disease progression (e.g., relapsing-remitting MS, secondary progressive MS, primary progressive MS, or progressive relapsing MS). Treatment according to the present invention is generally applicable to a subject having MS of any level or degree of disease activity. In certain embodiments, methods of the present invention are useful for treating MS is a subject having relapsing-remitting MS, wherein the methods comprise administering to the subject an effective amount of a compound that inhibits the activity of a HIF prolyl hydroxylase enzyme. In other embodiments, methods of the present invention are useful for treating MS in a subject having secondary progressive MS, wherein the methods comprise administering to the subject an effective amount of a compound that inhibits the activity of a HIF prolyl hydroxylase enzyme. In yet other embodiments, methods of the present invention are useful for treating MS in a subject having primary progressive MS, wherein the methods comprise administering to the subject an effective amount of a compound that inhibits the activity of a HIF prolyl hydroxylase enzyme. In yet further embodiments, methods of the present invention are useful for treating MS in a subject having progressive relapsing MS, wherein the methods comprise administering to the subject an effective amount of a compound that inhibits the activity of a HIF prolyl hydroxylase enzyme.

Methods for reducing or ameliorating one or more symptoms of MS are provided by the present invention, the methods comprising administering to a subject having MS an agent that inhibits HIF hydroxylase activity. In certain embodiments, the agent is a compound that inhibits HIF prolyl hydroxylase activity. In certain aspects, methods of the present invention are useful for reducing or ameliorating one or more of the following symptoms of MS: weakness or diminished dexterity in one or more limbs, muscle weakness, abnormal muscle spasms, or difficulty in moving (e.g., disturbance of gait); difficulties with coordination and balance (ataxia); problems in speech (dysarthria) or swallowing (dysphagia); visual problems (nystagmus, optic neuritis, or diplopia); fatigue and acute or chronic pain syndromes; and bladder and bowel difficulties.

In various embodiments, a compound used in the present methods is a structural mimetic of 2-oxoglutarate. In certain embodiments, the compound is a structural mimetic of 2-oxoglutarate that inhibits HIF prolyl hydroxylase activity competitively with respect to 2-oxoglutarate. In particular embodiments, compounds used in the present methods and medicaments provided herein are structural mimetics of 2-oxoglutarate, wherein the compound inhibits the target HIF prolyl hydroxylase enzyme competitively with respect to 2-oxoglutarate and noncompetitively with respect to iron.

In particular embodiments, compounds for use in the present invention include cyclic carboxamides, wherein the cyclic group is a carbocycle or a heterocycle. Therefore, in certain embodiments, the compounds used are carbocyclic carboxamides or heterocyclic carboxamides. In other embodiments, carbocyclic carboxamides for use in the present invention are naphthalene carboxamides. In yet other embodiments, heterocyclic carboxamides for use in the present invention are isoquinoline carboxamides, chromene carboxamides, thiochromene carboxamides, pyrrolopyridazine carboxamides, pyrrolopyridine carboxamides.

In certain embodiments, carbocyclic carboxamides for use in the present invention are hydroxy naphthalene carboxamides, oxo naphthalene carboxamides, and hydroxy oxo naphthalene carboxamides.

Heterocyclic carboxamides for use in the present invention include hydroxy isoquinoline carboxamides, hydroxy chromene carboxamides, oxo chromene carboxamides, hydroxy oxo chromene carboxamides, hydroxy thiochromene carboxamides, oxo thiochromene carboxamides, hydroxy oxo thiochromene carboxamides, oxo pyrrolopyridazine carboxamides, hydroxy pyrrolopyridazine carboxamides, hydroxy oxo pyrrolopyridazine carboxamides, and hydroxy pyrrolopyridine carboxamides.

In other embodiments, compounds for use in the present invention include variously substituted 4-hydroxy-isoquinoline-3-carbonyl glycines, 4-hydroxy-chromene-3-carbonyl glycines, 2-oxo-chromene-3-carbonyl glycines, 4-hydroxy-2-oxo-chromene-3-carbonyl glycines, 4-hydroxy-thiochromene-3-carbonyl glycines, 2-oxo-thiochromene-3-carbonyl glycines, 4-hydroxy-2-oxo-thiochromene-3-carbonyl glycines, 1-hydroxy-naphthalene-2-carbonyl glycines, 3-oxo-naphthalene-2-carbonyl glycines, 1-hydroxy-3-oxo-naphthalene-2-carbonyl glycines, 2-oxo-pyrrolopyridazine-3-carbonyl glycines, 4-hydroxy-pyrrolopyridazine-3-carbonyl glycines, 4-hydroxy-2-oxo-pyrrolopyridazine-3-carbonyl glycines, and 4-hydroxy-pyrrolo[2,3-c]pyridine-5-carbonyl glycines.

In particular embodiments, the compound used in the present invention is selected from the group consisting of [(1-Cyano-4-hydroxy-5-phenoxy-isoquinoline-3-carbonyl)-amino]-acetic acid (Compound A), [(1-Cyano-4-hydroxy-S-p-tolyloxy-isoquinoline-3-carbonyl)-amino]-acetic acid (Compound B), [(4-Hydroxy-1-pyridin-3-yl-8-p-tolyloxy-isoquinoline-3-carbonyl)-amino]-acetic acid (Compound C), {[7-(3-Fluoro-5-methoxy-phenoxy)-4-hydroxy-isoquinoline-3-carbonyl]-amino}-acetic acid (Compound D), {[4-Hydroxy-8-(3-methoxy-phenoxy)-isoquinoline-3-carbonyl]-amino}-acetic acid (Compound E), [(4-Hydroxy-7-phenoxy-isoquinoline-3-carbonyl)-amino]-acetic acid (Compound F), {[4-Hydroxy-2-oxo-1-(4-trifluoromethyl-benzyl)-1,2-dihydro-pyrrolo[1,2-b]pyridazine-3-carbonyl]-amino}-acetic acid (Compound G), (S)-2-{[6-Chloro-4-hydroxy-2-oxo-1-(4-trifluoromethyl-benzyl)-1,2-dihydro-pyrrolo[1,2-b]pyridazine-3-carbonyl]-amino}-propionic acid (Compound H), {[6-Chloro-1-(4-chloro-benzyl)-4-hydroxy-2-oxo-1,2-dihydro-pyrrolo[1,2-b]pyridazine-3-carbonyl]-amino}-acetic acid (Compound I), {[4-Hydroxy-2-oxo-7-(4-phenoxy-phenyl)-2H-chromene-3-carbonyl]-amino}-acetic acid (Compound J), [(6-Hexyloxy-4-hydroxy-2-oxo-2H-chromene-3-carbonyl)-amino]-acetic acid (Compound K), {[4-Hydroxy-7-(4-methoxy-phenyl)-2-oxo-2H-thiochromene-3-carbonyl]-amino}-acetic acid (Compound L), [(7-Butoxy-4-hydroxy-2-oxo-2H-thiochromene-3-carbonyl)-amino]-acetic acid (Compound M), [(7-Chloro-1-hydroxy-4,4-dimethyl-3-oxo-3,4-dihydro-naphthalene-2-carbonyl)-amino]-acetic acid (Compound N), {[7-Cyano-1-(2-fluoro-benzyl)-4-hydroxy-1H-pyrrolo[2,3-c]pyridine-5-carbonyl]-amino}-acetic acid (Compound O), [(1-Biphenyl-4-ylmethyl-7-cyano-4-hydroxy-1H-pyrrolo[2,3-c]pyridine-5-carbonyl)-amino]-acetic acid (Compound P), and {[2,3-Dichloro-7-cyano-4-hydroxy-1-(4-methoxy-benzyl)-1H-pyrrolo[2,3-c]pyridine-5-carbonyl]-amino}-acetic acid (Compound Q).

It is further contemplated that a compound for use in the present invention is a compound encompassed by one of Formulae I, Ia, Ib, Ic, and Id; Formula II; Formulae III and IIIa; Formulae IVA, IVB, IVC, and IVD; Formulae V, VA, VB, VC, and VD; Formula VI; Formula VII; Formula VIII; Formula IX; Formula X; and Formula XI. Each of these formulae are detailed, infra.

It is further contemplated that, in various embodiments, the methods of the present invention are used in combination with administration of one or more other therapeutic agents. Other therapeutic agents (subsequent or coordinate administration) for use in the present methods include interferon beta-1a (Avonex), interferon beta-1a (Rebif), interferon beta-1b (Betaseron, Extavia), glatiramer acetate (Copaxone), mitoxantrone (Novantrone), natalizumab (Tysabri), angiotensin-receptor blockers, or angiotensin converting-enzyme inhibitors.

These and other embodiments of the present invention will readily occur to those of skill in the art in light of the disclosure herein, and all such embodiments are specifically contemplated.

### BRIEF DESCRIPTION OF THE DRAWINGS

FIGS. 1A, 1B, and 1C set forth data showing methods and compounds of the present invention reduced disease severity in an EAE animal model of multiple sclerosis.

FIG. 2 sets forth data showing methods and compounds of the present invention reduced disease severity in an EAE animal model of multiple sclerosis.

FIGS. 3A and 3B set forth data showing methods and compounds of the present invention reduced disease severity in an EAE animal model of multiple sclerosis.

FIGS. 4A and 4B set forth data showing methods and compounds of the present invention reduced disease severity in an EAE animal model of multiple sclerosis.

FIG. 5 sets forth data showing methods and compounds of the present invention reduced disease severity in an EAE animal model of chronic progressive multiple sclerosis.

### DESCRIPTION OF THE INVENTION

Before the present compositions and methods are described, it is to be understood that the invention is not limited to the particular methodologies, protocols, cell lines, assays, and reagents described, as these may vary. It is also to be understood that the terminology used herein is intended to describe particular embodiments of the present invention, and is in no way intended to limit the scope of the present invention as set forth in the appended claims.

It must be noted that as used herein and in the appended claims, the singular forms “a,” “an,” and “the” include plural references unless context clearly dictates otherwise. Thus, for example, a reference to “a HIF-specific 2-oxoglutarate dioxygenase enzyme” may include a plurality of such enzymes; a reference to a “compound that inhibits the activity of a hypoxia-inducible factor prolyl hydroxylase enzyme” may be a reference to one or more compounds that inhibits the activity of a hypoxia-inducible factor prolyl hydroxylase enzyme, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications cited herein are incorporated herein by reference in their entirety for the purpose of describing and disclosing the methodologies, reagents, and tools reported in the publications that might be used in connection with the invention. Nothing herein is to be construed as an admission that the invention is not entitled to antedate such disclosure by virtue of prior invention.

The practice of the present invention will employ, unless otherwise indicated, conventional methods of chemistry, biochemistry, molecular biology, cell biology, genetics, immunology and pharmacology, within the skill of the art. Such techniques are explained fully in the literature. See, e.g., Gennaro, A. R., ed. (1990) Remington's Pharmaceutical Sciences, 18th ed., Mack Publishing Co.; Hardman, J. G., Limbird, L. E., and Gilman, A. G., eds. (2001) The Pharmacological Basis of Therapeutics, 10th ed., McGraw-Hill Co.; Colowick, S. et al., eds., Methods In Enzymology, Academic Press, Inc.; Weir, D. M., and Blackwell, C. C., eds. (1986) Handbook of Experimental Immunology, Vols. I-IV, Blackwell Scientific Publications; Maniatis, T. et al., eds. (1989) Molecular Cloning: A Laboratory Manual, 2nd edition, Vols. I-III, Cold Spring Harbor Laboratory Press; Ausubel, F. M. et al., eds. (1999) Short Protocols in Molecular Biology, 4th edition, John Wiley & Sons; Ream et al., eds. (1998) Molecular Biology Techniques: An Intensive Laboratory Course, Academic Press; Newton, C. R., and Graham, A., eds. (1997) PCR (Introduction to Biotechniques Series), 2nd ed., Springer Verlag.

The section headings are used herein for organizational purposes only, and are not to be construed as in any way limiting the subject matter described herein.

### Methods

The present invention relates in part to the discovery that stabilization of HIF-1α in a subject is effective at treating multiple sclerosis (MS).

In one embodiment, the present invention provides a method for treating MS in a subject, the method comprising administering to the subject an effective amount of an agent that stabilizes HIF-1α, thereby treating MS. In one embodiment, the agent that stabilizes HIF-1α is a compound that inhibits the activity of a HIF prolyl hydroxylase enzyme, and the present invention provides methods for treating MS in a subject, wherein the method comprises administering to the subject an effective amount of a compound that inhibits the activity of a HIF prolyl hydroxylase enzyme, thereby treating MS. The present invention also provides compounds for use in manufacturing a medicament for treating MS, wherein the compound inhibits the activity of a HIF prolyl hydroxylase enzyme. The present invention also provides compounds for use in manufacturing a medicament for treating MS in a subject, wherein the compound inhibits the activity of a HIF prolyl hydroxylase enzyme. In another embodiment, the present invention provides compounds for use in manufacturing a medicament for treating MS, wherein the compound inhibits the activity of HIF prolyl hydroxylase. The present invention also provides compounds for use in manufacturing a medicament for treating MS in a subject, wherein the compound inhibits the activity of HIF prolyl hydroxylase.

The disease course of MS varies, and is often associated with intermittent periods of disease remission and disease exacerbation (i.e., disease relapse). Several subtypes of MS have been described based on patterns of progression: relapsing-remitting MS (RRMS), characterized by unpredictable attacks (i.e., relapses) followed by periods of months to years of relative quiet remission with no new signs of disease activity; secondary progressive MS (SPMS), characterized by gradual progression of disability (e.g., neurologic decline) with or without superimposed relapses; primary progressive MS (PPMS), characterized by a gradual and continuous progression of disability from disease onset without superimposed relapses and often affects individuals who are older at time of disease onset; progressive relapsing MS (PRMS), characterized by gradual progression of disability from disease onset and later accompanied by one or more relapses. (See Lublin and Reingold (1996) Neurology 46:907-911.)

The present methods for treatment of MS in a subject are applicable to any subtype of MS or pattern of disease progression (e.g., relapsing-remitting MS, secondary progressive MS, primary progressive MS, or progressive relapsing MS). Treatment according to the present invention is generally applicable to a subject having MS of any level or degree of disease activity. In certain embodiments, methods of the present invention are useful for treating MS is a subject having relapsing-remitting MS, wherein the methods comprise administering to the subject an effective amount of a compound that inhibits the activity of a HIF prolyl hydroxylase enzyme. In other embodiments, methods of the present invention are useful for treating MS in a subject having secondary progressive MS, wherein the methods comprise administering to the subject an effective amount of a compound that inhibits the activity of a HIF prolyl hydroxylase enzyme. In yet other embodiments, methods of the present invention are useful for treating MS in a subject having primary progressive MS, wherein the methods comprise administering to the subject an effective amount of a compound that inhibits the activity of a HIF prolyl hydroxylase enzyme. In yet further embodiments, methods of the present invention are useful for treating MS in a subject having progressive relapsing MS, wherein the methods comprise administering to the subject an effective amount of a compound that inhibits the activity of a HIF prolyl hydroxylase enzyme.

Methods for treating a subject having MS, as provided in the present invention, can be applied at any point in the course of the disease. In certain embodiments, methods of the present invention are applied to a subject having MS during a time period of disease remission. In such embodiments, the present methods provide benefit by extending the time period of disease remission or by preventing, reducing, or delaying the onset of active disease or relapses. In other embodiments, methods of the present invention are applied to a subject having MS during a period of active disease (e.g., during a period of relapse). In such embodiments, the present methods provide benefit by reducing the duration of the period of active disease or relapse, reduce the severity of disease relapse, reduce or ameliorate one or more symptoms of MS, or treat MS.

MS can present with a variety of symptoms, including weakness or diminished dexterity in one or more limbs, muscle weakness, abnormal muscle spasms, or difficulty in moving (e.g., disturbance of gait); difficulties with coordination and balance (ataxia); problems in speech (dysarthria) or swallowing (dysphagia); visual problems (nystagmus, optic neuritis, or diplopia); fatigue and acute or chronic pain syndromes; and bladder and bowel difficulties. Methods for reducing or ameliorating one or more symptoms of MS are provided by the present invention, the methods comprising administering to a subject having MS an agent that inhibits HIF hydroxylase activity. In certain embodiments, the agent is a compound that inhibits HIF prolyl hydroxylase activity. In certain aspects, methods of the present invention are useful for reducing or ameliorating one or more of the following symptoms of MS: weakness or diminished dexterity in one or more limbs, muscle weakness, abnormal muscle spasms, or difficulty in moving (e.g., disturbance of gait); difficulties with coordination and balance (ataxia); problems in speech (dysarthria) or swallowing (dysphagia); visual problems (nystagmus, optic neuritis, or diplopia); fatigue and acute or chronic pain syndromes; and bladder and bowel difficulties.

As used herein, reducing or ameliorating one or more symptoms of MS refers to a qualitative or quantitative reduction in detectable symptoms, including but not limited to, a detectable effect on the rate of recovery from disease, length of time in remission, reduction in the number and/or severity of relapses, etc. Various methods have been described for assessing disease activity and severity of MS as well as response to treatment in subjects with MS. (See, e.g., Kurtzke (1983) Neurology 33:1444-1452.)

The methods of the present invention may be combined with the administration of one or more other therapeutic agents. In particular, the methods of the present invention may be combined with the administration of one or more therapeutic agents that may be effective in the treatment of MS. Such agents include: interferon beta-1a (Avonex); interferon beta-1a (Rebif); interferon beta-1b (Betaseron, Extavia); glatiramer acetate (Copaxone); mitoxantrone (Novantrone); natalizumab (Tysabri); angiotensin-receptor blockers; and angiotensin converting-enzyme inhibitors. Such agents may be administered in simultaneous, separate, or sequential (i.e., before or after) administration with the compounds of the present invention.

### Subjects

The present methods are directed to treating MS or to reducing or ameliorating one or more symptoms of MS in a subject in need, wherein the subject has MS. The subject can be a subject having any MS disease subtype or having MS with any pattern of disease progression. In certain embodiments, the subject has relapsing-remitting MS. In other embodiments, the subject has secondary progressive MS. In yet other embodiments, the subject has primary progressive MS. In further embodiments, the subject has progressive relapsing MS.

It is further contemplated that in certain embodiments, the subject is a subject with MS having one or more symptoms of MS, including, but not limited to, a subject having weakness or diminished dexterity in one or more limbs, muscle weakness, abnormal muscle spasms, or difficulty in moving (e.g., disturbance of gait); difficulties with coordination and balance (ataxia); problems in speech (dysarthria) or swallowing (dysphagia); visual problems (nystagmus, optic neuritis, or diplopia); fatigue and acute or chronic pain syndromes; and bladder and bowel difficulties.

### Compounds

Compounds for use in the methods or medicaments provided herein stabilize hypoxia-inducible factor alpha (HIFα) and are inhibitors of hypoxia-inducible factor (HIF) prolyl hydroxylase enzymes. A compound that inhibits the activity of HIF prolyl hydroxylase enzyme refers to any compound that reduces or otherwise moldulates the activity of at least one HIF prolyl hydroxylase enzyme. The term “HIF prolyl hydroxylase,” as used herein, refers to any enzyme that is capable of hydroxylating a proline residue within an alpha subunit of HIF. Such HIF prolyl hydroxylases include protein members of the EGL-9 (EGLN) 2-oxoglutarate- and iron-dependent dioxygenase family described by Taylor (2001) Gene 275:125-132; and characterized by Aravind and Koonin (2001) Genome Biol 2:RESEARCH0007; Epstein et al. (2001) Cell 107:43-54; and Bruick and McKnight (2001) Science 294:1337-1340.

Functionally, prolyl hydroxylase inhibitors for use in the methods of the present invention are defined by their ability to inhibit an activity of a 2-oxoglutarate dioxygenase enzyme, wherein the enzyme has specific activity toward hypoxia inducible factor. Such compounds are defined herein as prolyl hydroxylase inhibitors (PHIs). Preferably, the PHIs for use in the invention are small molecule compounds. A compound that inhibits the activity of a HIF prolyl hydroxylase enzyme refers to any compound that reduces or otherwise modulates the activity of at least one HIF prolyl hydroxylase enzyme. A compound may additionally show inhibitory activity toward one or more other 2-oxoglutarate- and iron-dependent dioxygenase enzymes, e.g. factor inhibiting HIF (FIH; GenBank Accession No. AAL27308), procollagen prolyl 4-hydroxylase (CP4H), etc.

It is contemplated herein that a “compound that inhibits HIF prolyl hydroxylase” suitable for use in the claimed methods can be any compound that inhibits HIF prolyl hydroxylase activity. As noted herein, the compound that inhibits HIF prolyl hydroxylase activity can be a structural mimetic of 2-oxoglutarate. In certain embodiments, the compound is a structural mimetic of 2-oxoglutarate that inhibits HIF prolyl hydroxylase activity competitively with respect to 2-oxoglutarate. In particular embodiments, compounds used in the present methods and medicaments provided herein are structural mimetics of 2-oxoglutarate, wherein the compound inhibits the target HIF prolyl hydroxylase enzyme competitively with respect to 2-oxoglutarate and noncompetitively with respect to iron.

### Cyclic Carboxamides

In specific structural embodiments, a compound suitable for use in the present invention is a cyclic carboxamide. The cyclic group is variously a carbocycle or a heterocycle. It is specifically contemplated that the cyclic group may contain additional substitutions at ring positions not occupied by the carboxamide moiety; for example, substitution of one or more atoms within the ring with a hydroxyl (—OH) or oxo (═O) group.

Accordingly, in certain embodiments, the compound is a carbocyclic carboxamide. The carbocyclic group can be a single ring group, e.g., a benzene, or can contain multiple condensed rings, e.g., a napthalene.

In particular embodiments, a compound suitable for use in the present invention is a heterocyclic carboxamide. In selected embodiments, the heterocycle can be a single ring, for example, a pyridine, a pyrimidine, or a pyridazine. In other embodiments, the specified heterocyclic structure is a multiple condensed ring, for example, a quinoline, a cinnoline, an isoquinoline, a pyrrolopyridine, a napthyridine, a β-carboline, a chromene (coumarin), or a thiochromene (thiocoumarin).

Carboxamide compounds particularly suitable for use in the present invention include carboxamides substituted at the amide to form a carbonyl glycine. Therefore, in certain embodiments, a compound for use in the present invention is a cyclic carbonyl glycine, and in particular, a carbocyclic carbonyl glycine or a heterocyclic carbonyl glycine. Specifically encompassed by the term “carbonyl glycine” are structural and functional analogs thereof, including, in particular, carbonyl glycineamides (wherein the carboxyl moiety on the glycine is replaced with carboxamide). Also encompassed are prodrugs thereof, such as carbonyl glycine esters (wherein the carboxyl moiety is esterified with a substituent such as an alkyl, e.g., methyl). In certain embodiments of the present invention, specific substitution at the a carbon of the glycine of a suitable heterocyclic carbonyl glycine compound results in replacement of the glycine with a comparable amino acid selected from the group consisting of alanine, valine, leucine, and isoleucine.

### Isoquinoline Carboxamides

In some embodiments of the present invention, a cyclic carboxamide for use in the present invention is a heterocyclic carboxamide and, more specifically, an isoquinoline carboxamide. The carboxamide can be positioned on the isoquinoline at any stereochemically appropriate point on the heterocycle. Isoquinoline carboxamides particularly suited for use in the present invention include isoquinoline-3-carboxamides.

Examples of such isoquinoline carboxamides include [(1-Cyano-4-hydroxy-5-phenoxy-isoquinoline-3-carbonyl)-amino]-acetic acid (Compound A), [(1-Cyano-4-hydroxy-5-p-tolyloxy-isoquinoline-3-carbonyl)-amino]-acetic acid (Compound B), [(4-Hydroxy-1-pyridin-3-yl-8-p-tolyloxy-isoquinoline-3-carbonyl)-amino]-acetic acid (Compound C), {[7-(3-Fluoro-5-methoxy-phenoxy)-4-hydroxy-isoquinoline-3-carbonyl]-amino}-acetic acid (Compound D), [4-Hydroxy-8-(3-methoxy-phenoxy)-isoquinoline-3-carbonyl)-amino]-acetic acid (Compound E), [(4-Hydroxy-7-phenoxy-isoquinoline-3-carbonyl)-amino]-acetic acid (Compound F), and other compounds readily identified by those skilled in the art, including those described and claimed in, e.g., U.S. Pat. No. 6,093,730 and in U.S. Patent Application Publication Nos. 2004/0254215 and 2007/0298104.

In other embodiments, an isoquinoline carboxamide according to the present invention is additionally substituted with a hydroxyl group; specifically a hydroxy isoquinoline carboxamide. The hydroxyl can be positioned on the isoquinoline at any stereochemically appropriate point on the heterocycle. Accordingly, isoquinoline carboxamides particularly suited for use in the present invention include 4-hydroxy-isoquinoline-3-carboxamides. Examples of such compounds include Compound A, Compound B, Compound C, Compound D, Compound E, Compound F, and other compounds readily identified by those skilled in the art, including those described and claimed in, e.g., U.S. Pat. No. 6,093,730 and U.S. Patent Application Publication Nos. 2004/0254215 and 2007/0298104.

In certain embodiments in which a compound of the invention is an isoquinoline carboxamide, the amide on the carboxamide moiety of the isoquinoline carboxamide is substituted to form a glycine, and the compound for use in the present invention is an isoquinoline carbonyl glycine. As the present invention particularly encompasses use of isoquinoline-3-carboxamides, isoquinoline-3-carbonyl glycines are specifically contemplated herein, as are more substituted examples thereof, including 4-hydroxy-isoquinoline-3-carbonyl glycines. Examples of such compounds include Compound A, Compound B, Compound C, Compound D, Compound E, Compound F, and other compounds readily identified by those skilled in the art, including those described and claimed in, e.g., U.S. Pat. No. 6,093,730 and U.S. Patent Application Publication Nos. 2004/0254215 and 2007/0298104.

### Chromene (Coumarin) Carboxamides

In other embodiments of the present invention, a cyclic carboxamide for use in the present invention is a heterocyclic carboxamide and, more specifically, a chromene carboxamide (coumarin). The carboxamide can be positioned on the chromene at any stereochemically appropriate point on the heterocycle. Chromene carboxamides particularly suited for use in the present invention include chromene-3-carboxamides. Examples of such compounds include {[4-Hydroxy-2-oxo-7-(4-phenoxy-phenyl)-2H-chromene-3-carbonyl]-amino}-acetic acid (Compound J), [(6-Hexyloxy-4-hydroxy-2-oxo-2H-chromene-3-carbonyl)-amino]-acetic acid (Compound K), and other compounds readily identified by those skilled in the art, including those described and claimed in, e.g., International Publication No. WO 2009/100250.

In some embodiments, the chromene carboxamide according to the present invention is additionally substituted with a hydroxyl group; specifically a hydroxy chromene carboxamide. The hydroxyl can be positioned on the chromene at any stereochemically appropriate point on the heterocycle. Chromene carboxamides particularly suited for use in the present invention include 4-hydroxy-chromene-3-carboxamides. Examples of such compounds include Compound J, Compound K, and other compounds readily identified by those skilled in the art, including those described and claimed in, e.g., International Publication No. WO 2009/100250.

In yet other embodiments, the chromene carboxamide according to the present invention is additionally substituted with an oxo group; specifically an oxo chromene carboxamide. The oxo can be positioned on the chromene at any stereochemically appropriate point on the heterocycle. Chromene carboxamides particularly suited for use in the present invention include 2-oxo-chromene-3-carboxamides. Examples of such compounds include Compound J, Compound K, and other compounds readily identified by those skilled in the art, including those described and claimed in, e.g., International Publication No. WO 2009/100250.

In specific embodiments, the chromene carboxamide according to the present invention is additionally substituted with a hydroxyl group and an oxo group; specifically a hydroxy oxo chromene carboxamide. The oxo and hydroxyl can be independently positioned on the chromene at any stereochemically appropriate point on the heterocycle. Chromene carboxamides particularly suited for use in the present invention include 4-hydroxy-2-oxo-chromene-3-carboxamides. Examples of such compounds include Compound J, Compound K, and other compounds readily identified by those skilled in the art, including those described and claimed in, e.g., International Publication No. WO 2009/100250.

In certain embodiments in which a compound of the invention is a chromene carboxamide, the amide on the carboxamide moiety of the chromene carboxamide is substituted to form a glycine, and the compound for use in the present invention is a chromene carbonyl glycine. As the present invention particularly encompasses use of chromene-3-carboxamides, chromene-3-carbonyl glycines are specifically contemplated herein, as are more substituted examples thereof, including 4-hydroxy-chromene-3-carbonyl glycines, 2-oxo-chromene-3-carbonyl glycines, and 4-hydroxy-2-oxo-chromene-3-carbonyl glycines. Examples of such compounds include Compound J, Compound K, and other compounds readily identified by those skilled in the art, including those described and claimed in, e.g., International Publication No. WO 2009/100250.

Thiochromene (Thiocoumarin) Carboxamides In other embodiments of the present invention, a cyclic carboxamide for use in the present invention is a heterocyclic carboxamide and, more specifically, a thiochromene carboxamide (thiocoumarin). The carboxamide can be positioned on the thiochromene at any stereochemically appropriate point on the heterocycle. Thiochromene carboxamides particularly suited for use in the present invention include thiochromene-3-carboxamides. Examples of such compounds include {[4-Hydroxy-7-(4-methoxy-phenyl)-2-oxo-2H-thiochromene-3-carbonyl]-amino}-acetic acid (Compound L), [(7-Butoxy-4-hydroxy-2-oxo-2H-thiochromene-3-carbonyl)-amino]-acetic acid (Compound M), and other compounds readily identified by those skilled in the art, including those described and claimed in, e.g., U.S. Provisional Application Ser. No. 61/114,971.

In some embodiments, the thiochromene carboxamide according to the present invention is additionally substituted with a hydroxyl group; specifically a hydroxy thiochromene carboxamide. The hydroxyl can be positioned on the thiochromene at any stereochemically appropriate point on the heterocycle. Thiochromene carboxamides particularly suited for use in the present invention include 4-hydroxy-thiochromene-3-carboxamides. Examples of such compounds include Compound L, Compound M, and other compounds readily identified by those skilled in the art, including those described and claimed in, e.g., U.S. Provisional Application Ser. No. 61/114,971.

In certain embodiments, the thiochromene carboxamide according to the present invention is additionally substituted with an oxo group; specifically an oxo thiochromene carboxamide. The oxo can be positioned on the thiochromene at any stereochemically appropriate point on the heterocycle. Thiochromene carboxamides particularly suited for use in the present invention include 2-oxo-thiochromene-3-carboxamides. Examples of such compounds include Compound L, Compound M, and other compounds readily identified by those skilled in the art, including those described and claimed in, e.g., U.S. Provisional Application Ser. No. 61/114,971.

In specific embodiments, the thiochromene carboxamide according to the present invention is additionally substituted with a hydroxyl group and an oxo group; specifically a hydroxy oxo thiochromene carboxamide. The oxo and hydroxyl can be independently positioned on the thiochromene at any stereochemically appropriate point on the heterocycle. Thiochromene carboxamides particularly suited for use in the present invention include 4-hydroxy-2-oxo-thiochromene-3-carboxamides. Examples of such compounds include Compound L, Compound M, and other compounds readily identified by those skilled in the art, including those described and claimed in, e.g., U.S. Provisional Application Ser. No. 61/114,971.

In certain embodiments in which a compound of the invention is a thiochromene carboxamide, the amide on the carboxamide moiety of the thiochromene carboxamide is substituted to form a glycine, and the compound for use in the present invention is a thiochromene carbonyl glycine. As the present invention particularly encompasses use of thiochromene-3-carboxamides, thiochromene-3-carbonyl glycines are specifically contemplated herein, as are more substituted examples thereof, including 4-hydroxy-thiochromene-3-carbonyl glycines, 2-oxo-thiochromene-3-carbonyl glycines, and 4-hydroxy-2-oxo-thiochromene-3-carbonyl glycines. Examples of such compounds include Compound L, Compound M, and other compounds readily identified by those skilled in the art, including those described and claimed in, e.g., U.S. Provisional Application Ser. No. 61/114,971.

### Naphthalenone Carboxamides

In one embodiment of the present invention, a cyclic carboxamide for use in the present invention is a carbocyclic carboxamide and, more specifically, a naphthalene carboxamide. The carboxamide can be positioned on the naphthalene at any stereochemically appropriate point on the carbocycle. Naphthalene carboxamides particularly suited for use in the present invention include naphthalene-2-carboxamides. Examples of such compounds include [(7-Chloro-1-hydroxy-4,4-dimethyl-3-oxo-3,4-dihydro-naphthalene-2-carbonyl)-amino]-acetic acid (Compound N) and other compounds readily identified by those skilled in the art, including those described and claimed in, e.g., International Publication No. WO 2008/076427.

In some embodiments, the naphthalene carboxamide according to the present invention is additionally substituted with a hydroxyl group; specifically a hydroxy naphthalene carboxamide. The hydroxyl can be positioned on the naphthalene at any stereochemically appropriate point on the carbocycle. Naphthalene carboxamides particularly suited for use in the present invention include 1-hydroxy-naphthalene-2-carboxamides. Examples of such compounds include Compound N and other compounds readily identified by those skilled in the art, including those described and claimed in, e.g., International Publication No. WO 2008/076427.

In other embodiments, the naphthalene carboxamide according to the present invention is additionally substituted with an oxo group; specifically an oxo naphthalene carboxamide. The oxo can be positioned on the naphthalene at any stereochemically appropriate point on the carbocycle. Naphthalene carboxamides particularly suited for use in the present invention include 3-oxo-naphthalene-2-carboxamides. Examples of such compounds include Compound N and other compounds readily identified by those skilled in the art, including those described and claimed in, e.g., International Publication No. WO 2008/076427.

In specific embodiments, the naphthalene carboxamide according to the present invention is additionally substituted with an oxo group and a hydroxyl group; specifically a hydroxy oxo naphthalene carboxamide. The oxo and hydroxyl can be independently positioned on the naphthalene at any stereochemically appropriate point on the carbocycle. Naphthalene carboxamides particularly suited for use in the present invention include 1-hydroxy-3-oxo-naphthalene-2-carboxamide. Examples of such compounds include Compound N and other compounds readily identified by those skilled in the art, including those described and claimed in, e.g., International Publication No. WO 2008/076427.

In certain embodiments in which a compound of the invention is a naphthalene carboxamide, the amide on the carboxamide moiety of the naphthalene carboxamide is substituted to form a glycine, and the compound for use in the present invention is a naphthalene carbonyl glycine. As the present invention particularly encompasses use of naphthalene-2-carboxamides, naphthalene-2-carbonyl glycines are specifically contemplated herein, as are more substituted examples thereof, including 1-hydroxy-naphthalene-2-carbonyl glycines, 3-oxo-naphthalene-2-carbonyl glycines, and 1-hydroxy-3-oxo-naphthalene-2-carbonyl glycines. Examples of such compounds include Compound N and other compounds readily identified by those skilled in the art, including those described and claimed in, e.g., International Publication No. WO 2008/076427.

### Pyrrolopyridazinone Carboxamides

In another embodiment of the present invention, a cyclic carboxamide for use in the present invention is a heterocyclic carboxamide and, more specifically, a pyrrolopyridazine carboxamide. The carboxamide can be positioned on the pyrrolopyridazine at any stereochemically appropriate point on the heterocycle. Pyrrolopyridazine carboxamides particularly suited for use in the present invention include pyrrolopyridazine-3-carboxamides. Examples of such compounds include {[4-Hydroxy-2-oxo-1-(4-trifluoromethyl-benzyl)-1,2-dihydro-pyrrolo[1,2-b]pyridazine-3-carbonyl]-amino}-acetic acid (Compound G), (S)-2-[6-Chloro-4-hydroxy-2-oxo-1-(4-trifluoromethyl-benzyl)-1,2-dihydro-pyrrolo[1,2-b]pyridazine-3-carbonyl)-amino]-propionic acid (Compound H), {[6-Chloro-1-(4-chloro-benzyl)-4-hydroxy-2-oxo-1,2-dihydro-pyrrolo[1,2-b]pyridazine-3-carbonyl]-amino}-acetic acid (Compound I), and other compounds readily identified by those skilled in the art, including those described and claimed in, e.g., International Application No. PCT/US09/54473.

In some embodiments, the pyrrolopyridazine carboxamide according to the present invention is additionally substituted with an oxo group; specifically an oxo pyrrolopyridazine carboxamide. The oxo can be positioned on the pyrrolopyridazine at any stereochemically appropriate point on the heterocycle. Pyrrolopyridazine carboxamides particularly suited for use in the present invention include 2-oxo-pyrrolopyridazine-3-carboxamides. Examples of such compounds include Compound G, Compound H, Compound I, and other compounds readily identified by those skilled in the art, including those described and claimed in, e.g., International Application No. PCT/US09/54473.

In certain embodiments, the pyrrolopyridazine carboxamide according to the present invention is additionally substituted with a hydroxyl group; specifically a hydroxy pyrrolopyridazine carboxamide.

The hydroxyl can be positioned on the pyrrolopyridazine at any stereochemically appropriate point on the heterocycle. Pyrrolopyridazine carboxamides particularly suited for use in the present invention include 4-hydroxy-pyrrolopyridazine-3-carboxamides. Examples of such compounds include Compound G, Compound H, Compound I, and other compounds readily identified by those skilled in the art, including those described and claimed in, e.g., International Application No. PCT/US09/54473.

In particular embodiments, the pyrrolopyridazine carboxamide according to the present invention is additionally substituted with a hydroxyl group and an oxo group; specifically a hydroxy oxo pyrrolopyridazine carboxamide. The oxo and hydroxyl can be independently positioned on the pyrrolopyridazine at any stereochemically appropriate point on the heterocycle. Pyrrolopyridazine carboxamides particularly suited for use in the present invention include 4-hydroxy-2-oxo-pyrrolopyridazine-3-carboxamides. Examples of such compounds include Compound G, Compound H, Compound I, and other compounds readily identified by those skilled in the art, including those described and claimed in, e.g., International Application No. PCT/US09/54473.

In certain embodiments in which a compound of the invention is a pyrrolopyridazine carboxamide, the amide on the carboxamide moiety of the pyrrolopyridazine carboxamide is substituted to form a glycine, and the compound for use in the present invention is a pyrrolopyridazine carbonyl glycine. As the present invention particularly encompasses use of pyrrolopyridazine-3-carboxamides, pyrrolopyridazine-3-carbonyl glycines are specifically contemplated herein, as are more substituted examples thereof, including 2-oxo-pyrrolopyridazine-3-carbonyl glycines, 4-hydroxy-pyrrolopyridazine-3-carbonyl glycines, and 4-hydroxy-2-oxo-pyrrolopyridazine-3-carbonyl glycines. Examples of such compounds include Compound G, Compound H, Compound I, and other compounds readily identified by those skilled in the art, including those described and claimed in, e.g., International Application No. PCT/US09/54473.

### Pyrrolopyridine Carboxamides

In other embodiments of the present invention, a cyclic carboxamide for use in the present invention is a heterocyclic carboxamide and, more specifically, a pyrrolopyridine carboxamide. The carboxamide can be positioned on the pyrrolopyridine at any stereochemically appropriate point on the heterocycle. Pyrrolopyridine carboxamides particularly suited for use in the present invention include pyrrolo[2,3-c]pyridine-5-carboxamides. Examples of such compounds include {[7-Cyano-1-(2-fluoro-benzyl)-4-hydroxy-1H-pyrrolo[2,3-c]pyridine-5-carbonyl]amino}-acetic acid (Compound O), [(1-Biphenyl-4-ylmethyl-7-cyano-4-hydroxy-1H-pyrrolo[2,3-c]pyridine-5-carbonyl)-amino]-acetic acid (Compound P), {[2,3-Dichloro-7-cyano-4-hydroxy-1-(4-methoxy-benzyl)-1H-pyrrolo[2,3-c]pyridine-5-carbonyl]-amino}-acetic acid (Compound Q), and other compounds readily identified by those skilled in the art, including those described and claimed in, e.g., U.S. Patent Application No. 2008/0004309.

In some embodiments, the pyrrolopyridine carboxamide according to the present invention is additionally substituted with a hydroxyl group; specifically a hydroxy pyrrolopyridine carboxamide. The hydroxyl can be positioned on the pyrrolopyridine at any stereochemically appropriate point on the heterocycle. Pyrrolopyridine carboxamides particularly suited for use in the present invention include 4-hydroxy-pyrrolo[2,3-c]pyridine-5-carboxamides. Examples of such compounds include Compound O, Compound P, Compound Q, and other compounds readily identified by those skilled in the art, including those described and claimed in, e.g., U.S. Patent Application No. 2008/0004309.

In certain embodiments in which a compound of the invention is a pyrrolopyridine carboxamide, the amide on the carboxamide moiety of the pyrrolopyridine carboxamide is substituted to form a glycine, and the compound for use in the present invention is a pyrrolopyridine carbonyl glycine. As the present invention particularly encompasses use of pyrrolo[2,3-c]pyridine-5-carboxamides, pyrrolo[2,3-c]pyridine-5-carbonyl glycines are specifically contemplated herein, as are more substituted examples thereof, including 4-hydroxy-pyrrolo[2,3-c]pyridine-5-carbonyl glycines. Examples of such compounds include Compound O, Compound P, Compound Q, and other compounds readily identified by those skilled in the art, including those described and claimed in, e.g., U.S. Patent Application No. 2008/0004309.

Exemplary compounds for use in the present invention include [(1-Cyano-4-hydroxy-5-phenoxy-isoquinoline-3-carbonyl)-amino]-acetic acid (Compound A), [(1-Cyano-4-hydroxy-5-p-tolyloxy-isoquinoline-3-carbonyl)-amino]-acetic acid (Compound B), [(4-Hydroxy-1-pyridin-3-yl-8-p-tolyloxy-isoquinoline-3-carbonyl)-amino]-acetic acid (Compound C), {[7-(3-Fluoro-5-methoxy-phenoxy)-4-hydroxy-isoquinoline-3-carbonyl]-amino}-acetic acid (Compound D), {[4-Hydroxy-8-(3-methoxy-phenoxy)-isoquinoline-3-carbonyl]-amino}-acetic acid (Compound E), [(4-Hydroxy-7-phenoxy-isoquinoline-3-carbonyl)-amino]-acetic acid (Compound F), {[4-Hydroxy-2-oxo-1-(4-trifluoromethyl-benzyl)-1,2-dihydro-pyrrolo[1,2-b]pyridazine-3-carbonyl]-amino}-acetic acid (Compound G), (S)-2-{[6-Chloro-4-hydroxy-2-oxo-1-(4-trifluoromethyl-benzyl)-1,2-dihydro-pyrrolo[1,2-b]pyridazine-3-carbonyl]-amino}-propionic acid (Compound H), {[6-Chloro-1-(4-chloro-benzyl)-4-hydroxy-2-oxo-1,2-dihydro-pyrrolo[1,2-b]pyridazine-3-carbonyl]-amino}-acetic acid (Compound I), {[4-Hydroxy-2-oxo-7-(4-phenoxy-phenyl)-2H-chromene-3-carbonyl]-amino}-acetic acid (Compound J), [(6-Hexyloxy-4-hydroxy-2-oxo-2H-chromene-3-carbonyl)-amino]-acetic acid (Compound K), {[4-Hydroxy-7-(4-methoxy-phenyl)-2-oxo-2H-thiochromene-3-carbonyl]-amino}-acetic acid (Compound L), [(7-Butoxy-4-hydroxy-2-oxo-2H-thiochromene-3-carbonyl)-amino]-acetic acid (Compound M), [(7-Chloro-1-hydroxy-4,4-dimethyl-3-oxo-3,4-dihydro-naphthalene-2-carbonyl)-amino]-acetic acid (Compound N), ([7-Cyano-1-(2-fluoro-benzyl)-4-hydroxy-1H-pyrrolo[2,3-c]pyridine-5-carbonyl)-amino]-acetic acid (Compound O), [(1-Biphenyl-4-ylmethyl-7-cyano-4-hydroxy-1H-pyrrolo[2,3-c]pyridine-5-carbonyl)-amino]-acetic acid (Compound P), and {[2,3-Dichloro-7-cyano-4-hydroxy-1-(4-methoxy-benzyl)-1H-pyrrolo[2,3-c]pyridine-5-carbonyl]-amino}-acetic acid (Compound Q).

Additional examples of compounds suitable for use according to the present invention are presented below.

In other particular embodiments, a compound of the invention is a heterocyclic carbonyl glycine of formula VI.

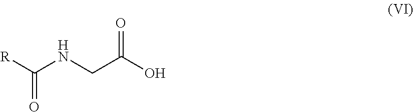

wherein R is a heterocyclic moiety.

In certain embodiments, the heterocyclic carbonyl glycine is a quinoline carboxamide, an isoquinoline carboxamide, a pyridine carboxamide, a cinnoline carboxamide, or a beta-carboline carboxamide. Heterocyclic carbonyl glycines effectively stabilize HIFα. In specific aspects, a compound of the invention is a compound that inhibits prolyl hydroxylase activity (e.g., a prolyl hydroxylase inhibitor). In more particular aspects, a compound of the invention is a compound that inhibits HIF prolyl hydroxylase activity. Prolyl hydroxylase inhibitors (PHIS) specifically contemplated for use in the present methods are described, e.g., in Majamaa et al., supra; Kivirikko and Myllyharju (1998) Matrix Biol 16:357-368; Bickel et al. (1998) Hepatology 28:404-411; Friedman et al. (2000) Proc Natl Acad Sci USA 97:4736-4741; Franklin (1991) Biochem Soc Trans 19):812 815; Franklin et al. (2001) Biochem J 353:333-338. Examples of compounds that may be used in the methods and medicaments provided herein can be found, e.g., in Majamaa et al. (1984) Eur. J. Biochem. 138:239-245; Majamaa et al. (1985) Biochem. J. 229:127-133; Kivirikko, and Myllyharju (1998) Matrix Biol. 16:357-368; Bickel et al. (1998) Hepatology 28:404-411; Friedman et al. (2000) Proc. Natl. Acad. Sci. USA 97:4736-4741; Franklin (1991) Biochem. Soc. Trans. 19):812-815; and Franklin et al. (2001) Biochem. J. 353:333-338. Additionally, compounds that inhibit HIF prolyl hydroxylase enzyme activity or that stabilize HIFα have been described in, e.g., International Publication Nos. WO 2003/049686, WO 2002/074981, WO 03/053977, WO 2003/080566, WO 2004/108121, WO 2004/108681, WO 2006/094292, WO 2007/038571, WO 2007/090068, WO 2007/070359, WO 2007/103905, and WO 2007/115315. All compounds listed in the above-patent and patent applications are hereby incorporated into the present application by reference herein in their entirety.

In various embodiments, a compound for use in the present methods is a heterocyclic carbonyl glycine, in particular, a heterocyclic carbonyl glycine of Formula VI. In certain embodiments, the compound used in the present methods is a compound selected from the group consisting of the compounds of Formula I, Formula II, Formula III, Formula IV, Formula V, and Formula VI. Formula I includes, but is not limited to, compounds of Formulae Ia, Ib, Ic, and Id. Formula III includes, but is not limited to, the compounds of Formula Ma. Formula IV includes, but is not limited to, compounds of Formulae IVA, IVB, IVC, and IVD. Formula V includes, but is not limited to, compounds of Formulae VA, VB, VC, and VD.

As stated, supra, compounds suitable for use in the present invention include isoquinoline carboxamides. In various embodiments, isoquinoline carboxamides according to the present invention are isoquinoline-3-carboxamides. In one embodiment, a compound for use in the methods of the present invention is [(1-Cyano-4-hydroxy-5-phenoxy-isoquinoline-3-carbonyl)-amino]-acetic acid (Compound A), [(1-Cyano-4-hydroxy-5-p-tolyloxy-isoquinoline-3-carbonyl)-amino]-acetic acid (Compound B), [(4-Hydroxy-1-pyridin-3-yl-8-p-tolyloxy-isoquinoline-3-carbonyl)-amino]-acetic acid (Compound C), {[7-(3-Fluoro-5-methoxy-phenoxy)-4-hydroxy-isoquinoline-3-carbonyl]-amino}-acetic acid (Compound D), {[4-Hydroxy-8-(3-methoxy-phenoxy)-isoquinoline-3-carbonyl]-amino}-acetic acid (Compound E), or [(4-Hydroxy-7-phenoxy-isoquinoline-3-carbonyl)-amino]-acetic acid (Compound F).

In certain embodiments, compounds used in the methods of the invention are heterocyclic carboxamides selected from a compound of the formula (I)

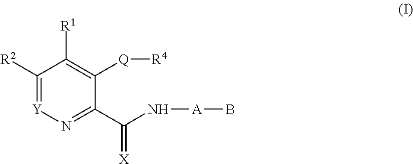

wherein
A is 1,2-arylidene, 1,3-arylidene, 1,4-arylidene; or (C₁-C₄)-alkylene, optionally substituted by one or two halogen, cyano, nitro, trifluoromethyl, (C₁-C₆)-alkyl, (C₁-C₆)-hydroxyalkyl, (C₁-C₆)-alkoxy, —O—[CH₂]ₓ—C_{f}H_{(2f+1-g)}Hal_{g}, (C₁-C₆)-fluoroalkoxy, (C₁-C₈)-fluoroalkenyloxy, (C₁-C₈)-fluoroalkynyloxy, —OCF₂Cl, —O—CF₂—CHFCl; (C₁-C₆)-alkylmercapto, (C₁-C₆)-alkylsulfinyl, (C₁-C₆)-alkylsulfonyl, (C₁-C₆)-alkylcarbonyl, (C₁-C₆)-alkoxycarbonyl, carbamoyl, N—(C₁-C₄)-alkylcarbamoyl, N,N-di-(C₁-C₄)-alkylcarbamoyl, (C₁-C₆)-alkylcarbonyloxy, (C₃-C₈)-cycloalkyl, phenyl, benzyl, phenoxy, benzyloxy, anilino, N-methylanilino, phenylmercapto, phenylsulfonyl, phenylsulfinyl, sulfamoyl, N—(C₁-C₄)-alkylsulfamoyl, N,N-di-(C₁-C₄)-alkylsulfamoyl; or by a substituted (C₆-C₁₂)-aryloxy, (C₇-C₁₁)-aralkyloxy, (C₆-C₁₂)-aryl, (C₇-C₁₁)-aralkyl radical, which carries in the aryl moiety one to five identical or different substituents selected from halogen, cyano, nitro, trifluoromethyl, (C₁-C₆)-alkyl, (C₁-C₆)-alkoxy, —O—[CH₂]ₓ—C_{f}H_{(2f+1-g)}Hal_{g}, —OCF₂Cl, —O—CF₂—CHFCl, (C₁-C₆)-alkylmercapto, (C₁-C₆)-alkylsulfinyl, (C₁-C₆)-alkylsulfonyl, (C₁-C₆)-alkylcarbonyl, (C₁-C₆)-alkoxycarbonyl, carbamoyl, N—(C₁-C₄)-alkylcarbamoyl, N,N-di-(C₁-C₄)-alkylcarbamoyl, (C₁-C₆)-alkylcarbonyloxy, (C₃-C₈)-cycloalkyl, sulfamoyl, N—(C₁-C₄)-alkylsulfamoyl, N,N-di-(C₁-C₄)-alkylsulfamoyl; or wherein A is —CR⁵R⁶ and R⁵ and R⁶ are each independently selected from hydrogen, (C₁-C₆)-alkyl, (C₃-C₇)-cycloalkyl, aryl, or a substituent of the α-carbon atom of an α-amino acid, wherein the amino acid is a natural L-amino acid or its D-isomer.
B is —CO₂H, —NH₂, —NHSO₂CF₃, tetrazolyl, imidazolyl, 3-hydroxyisoxazolyl, —CONHCOR′″, —CONHSOR′″, CONHSO₂R′″, where R′″ is aryl, heteroaryl, (C₃-C₇)-cycloalkyl, or (C₁-C₄)-alkyl, optionally monosubstituted by (C₆-C₁₂)-aryl, heteroaryl, OH, SH, (C₁-C₄)-alkyl, (C₁-C₄)-alkoxy, (C₁-C₄)-thioalkyl, (C₁-C₄)-sulfinyl, (C₁-C₄)-sulfonyl, CF₃, Cl, Br, F, I, NO₂, —COOH, (C₂-C₅)-alkoxycarbonyl, NH₂, mono-(C₁-C₄-alkyl)-amino, di-(C₁-C₄-alkyl)-amino, or (C₁-C₄)-perfluoroalkyl; or wherein B is a CO₂-G carboxyl radical, where G is a radical of an alcohol G-OH in which G is selected from (C₁-C₂₀)-alkyl radical, (C₃-C₈) cycloalkyl radical, (C₂-C₂₀)-alkenyl radical, (C₃-C₈)-cycloalkenyl radical, retinyl radical, (C₂-C₂₀)-alkynyl radical, (C₄-C₂₀)-alkenynyl radical, where the alkenyl, cycloalkenyl, alkynyl, and alkenynyl radicals contain one or more multiple bonds; (C₆-C₁₆)-carbocyclic aryl radical, (C₇-C₁₆)-carbocyclic aralkyl radical, heteroaryl radical, or heteroaralkyl radical, wherein a heteroaryl radical or heteroaryl moiety of a heteroaralkyl radical contains 5 or 6 ring atoms; and wherein radicals defined for G are substituted by one or more hydroxyl, halogen, cyano, trifluoromethyl, nitro, carboxyl, (C₁-C₁₂)-alkyl, (C₃-C₈)-cycloalkyl, (C₅-C₈)-cycloalkenyl, (C₆-C₁₂)-aryl, (C₇-C₁₆)-aralkyl, (C₂-C₁₂)-alkenyl, (C₂-C₁₂)-alkynyl, (C₁-C₁₂)-alkoxy, (C₁-C₁₂)-alkoxy-(C₁-C₁₂)-alkyl, (C₁-C₁₂)-alkoxy-(C₁-C₁₂)-alkoxy, (C₆-C₁₂)-aryloxy, (C₇-C₁₆)-aralkyloxy, (C₁-C₈)-hydroxyalkyl, —O—[CH₂]ₓ—C_{f}H_{(2f+1-g)}—F_{g}, —OCF₂Cl, —OCF₂—CHFCl, (C₁-C₁₂)-alkylcarbonyl, (C₃-C₈)-cycloalkylcarbonyl, (C₆-C₁₂)-arylcarbonyl, (C₇-C₁₆)-aralkylcarbonyl, cinnamoyl, (C₂-C₁₂)-alkenylcarbonyl, (C₂-C₁₂)-alkynylcarbonyl, (C₁-C₁₂)-alkoxycarbonyl, (C₁-C₁₂)-alkoxy-(C₁-C₁₂)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₁₂)-alkenyloxycarbonyl, (C₂-C₁₂)-alkynyloxycarbonyl, acyloxy, (C₁-C₁₂)-alkoxycarbonyloxy, (C₁-C₁₂)-alkoxy-(C₁-C₁₂)-alkoxycarbonyloxy, (C₆-C₁₂)-aryloxycarbonyloxy, (C₇-C₁₆) aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N,N-di(C₁-C₁₂)-alkylcarbamoyl, N—(C₃-C₈)-cycloalkyl-carbamoyl, N—(C₆-C₁₆)-arylcarbamoyl, N—(C₇-C₁₆)-aralkylcarbamoyl, N—(C₁-C₁₀)-alkyl-N—(C₆-C₁₆)-arylcarbamoyl, N—(C₁-C₁₀)-alkyl-N—(C₇-C₁₆)-aralkylcarbamoyl, N—((C₁-C₁₀)-alkoxy-(C₁-C₁₀)-alkyl)-carbamoyl, N—((C₆-C₁₂)-aryloxy-(C₁-C₁₀)-alkyl)-carbamoyl, N—((C₇-C₁₆)-aralkyloxy-(C₁-C₁₀)-alkyl)-carbamoyl, N—(C₁-C₁₀)-alkyl-N—((C₁-C₁₀)-alkoxy-(C₁-C₁₀)-alkyl)-carbamoyl, N—(C₁-C₁₀)-alkyl-N—((C₆-C₁₆)-aryloxy-(C₁-C₁₀)-alkyl)-carbamoyl, N—(C₁-C₁₀)-alkyl-N—((C₇-C₁₆)-aralkyloxy-(C₁-C₁₀)-alkyl)-carbamoyl, carbamoyloxy, N—(C₁-C₁₂)-alkylcarbamoyloxy, N,N-di-(C₁-C₁₂)-alkylcarbamoyloxy, N—(C₃-C₈)-cycloalkylcarbamoyloxy, N—(C₆-C₁₂)-arylcarbamoyloxy, N—(C₇-C₁₆)-aralkylcarbamoyloxy, N—(C₁-C₁₀)-alkyl-N—(C₆-C₁₂)-arylcarbamoyloxy, N(C₁-C₁₀)-alkyl-N—(C₇-C₁₆)-aralkylcarbamoyloxy, N—((C₁-C₁₀)-alkyl)-carbamoyloxy, N—(C₆-C₁₂)-aryloxy-(C₁-C₁₀)-alkyl)-carbamoyloxy, N—((C₇-C₁₆)-aralkyloxy-(C₁-C₁₀)-alkyl)-carbamoyloxy, N—(C₁-C₁₀)-alkyl-N—((C₁-C₁₀)-alkoxy-(C₁-C₁₀)-alkyl)-carbamoyloxy, N—(C₁-C₁₀)-alkyl-N—(C₆-C₁₂)-aryloxy-(C₁-C₁₀)-alkyl)-carbamoyloxy, N—(C₁-C₁₀)-alkyl-N—((C₇-C₁₆)-aralkyloxy-(C₁-C₁₀)-alkyl)-carbamoyloxy, amino, (C₁-C₁₂)-alkylamino, di-(C₁-C₁₂)-alkylamino, (C₃-C₈)-cycloalkylamino, (C₂-C₁₂)-alkenylamino, (C₂-C₁₂)-alkynylamino, N—(C₆-C₁₂)-arylamino, N—(C—C₁₁)-aralkylamino, N-alkyl-aralkylamino, N-alkyl-arylamino, (C₁-C₁₂)-alkoxyamino, (C₁-C₁₂)-alkoxy-N—(C₁-C₁₀)-alkylamino, (C₁-C₁₂)-alkylcarbonylamino, (C₃-C₈)-cycloalkylcarbonylamino, (C₆-C₁₂) arylcarbonylamino, (C₇-C₁₆)-aralkylcarbonylamino, (C₁-C₁₂)-alkylcarbonyl-N—(C₁-C₁₀)-alkylamino, (C₃-C₈)-cycloalkylcarbonyl-N—(C₁-C₁₀)-alkylamino, (C₆-C₁₂)-arylcarbonyl-N—(C₁-C₁₀)alkylamino, (C₇-C₁₁)-aralkylcarbonyl-N—(C₁-C₁₀)-alkylamino, (C₁-C₁₂)-alkylcarbonylamino-(C₁-C₈)-alkyl, (C₃-C₈)-cycloalkylcarbonylamino-(C₁-C₈)alkyl, (C₆-C₁₂)-arylcarbonylamino-(C₁-C₈)-alkyl, (C₇-C₁₂)-aralkylcarbonylamino(C₁-C₈)-alkyl, amino-(C₁-C₁₀)-alkyl, N—(C₁-C₁₀) alkylamino-(C₁-C₁₀)-alkyl, (C₃-C₈)-cycloalkylamino-(C₁-C₁₀)-alkyl, (C₁-C₁₂)-alkylmercapto, (C₁-C₁₂)-alkylsulfinyl, (C₁-C₁₂)-alkylsulfonyl, (C₆-C₁₆)-arylmercapto, (C₆-C₁₆)-arylsulfinyl, (C₆-C₁₂)-arylsulfonyl, (C₇-C₁₆)-aralkylmercapto, (C₇-C₁₆)-aralkylsulfinyl, (C₇-C₁₆)-aralkylsulfonyl, sulfamoyl, N—(C₁-C₁₀)-alkylsulfamoyl, N,N-di(C₁-C₁₀)-alkylsulfamoyl, (C₃-C₈)-cycloalkylsulfamoyl, N—(C₆-C₁₂)-alkylsulfamoyl, N—(C₇-C₁₆)-aralkylsulfamoyl, N—(C₁-C₁₀)-alkyl-N—(C₆-C₁₂)-arylsulfamoyl, N—(C₁-C₁₀)-alkyl-N—(C₇-C₁₆)-aralkylsulfamoyl, (C₁-C₁₀)-alkylsulfonamido, N—(C₁-C₁₀)-alkyl)-(C₁-C₁₀)-alkylsulfonamido, (C₇-C₁₆)-aralkylsulfonamido, or N—((C₁-C₁₀)-alkyl-(C₇-C₁₆)-aralkylsulfonamido; wherein radicals which are aryl or contain an aryl moiety, may be substituted on the aryl by one to five identical or different hydroxyl, halogen, cyano, trifluoromethyl, nitro, carboxyl, (C₁-C₁₂)-alkyl, (C₃-C₈)-cycloalkyl, (C₆-C₁₂)-aryl, (C₇-C₁₆)-aralkyl, (C₁-C₁₂)-alkoxy, (C₁-C₁₂)-alkoxy-(C₁-C₁₂)allyl, (C₁-C₁₂)-alkoxy-(C₁ C₁₂)alkoxy, (C₆-C₁₂)-aryloxy, (C₇-C₁₆)-aralkyloxy, (C₁-C₈)-hydroxyalkyl, (C₁-C₁₂)-alkylcarbonyl, (C₃-C₈)-cycloalkyl-carbonyl, (C₆-C₁₂)-arylcarbonyl, (C₇-C₁₆) aralkylcarbonyl, (C₁-C₁₂)-alkoxycarbonyl, (C₁-C₁₂)-alkoxy-(C₁-C₁₂)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₁₂)-alkenyloxycarbonyl, (C₂-C₁₂)-alkynyloxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₁-C₁₂)-alkoxy-(C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N,N-di-(C₁-C₁₂)-alkylcarbamoyl, N—(C₃-C₈)-cycloalkylcarbamoyl, N—(C₆-C₁₂)-arylcarbamoyl, N—(C₇-C₁₆)-aralkylcarbamoyl, N—(C₁-C₁₀)-alkyl-N—(C₆-C₁₂)-arylcarbamoyl, N—(C₁-C₁₀)-alkyl-N—(C₇-C₁₆)-aralkylcarbamoyl, N—((C₁-C₁₀)-alkoxy-(C₁-C₁₀)-alkyl)-carbamoyl, N—(C₆-C₁₂)-aryloxy-(C₁-C₁₀)-alkyl)-carbamoyl, N—(C₇-C₁₆)-aralkyloxy-(C₁-C₁₀)-alkyl)-carbamoyl, N—(C₁-C₁₀)-alkyl-N—((C₁-C₁₀)-alkoxy-(C₁-C₁₀)-alkyl)-carbamoyl, N—(C₁-C₁₀)-alkyl-N—((C₆-C₁₂)-aryloxy-(C₁-C₁₀)-alkyl)-carbamoyl, N—(C₁-C₁₀)-alkyl-N—((C₇-C₁₆)-aralkyloxy-(C₁-C₁₀)-alkyl)-carbamoyl, carbamoyloxy, N—(C₁-C₁₂)-alkylcarbamoyloxy, N,N-di-(C₁-C₁₂)-alkylcarbamoyloxy, N—(C₃-C₈)-cycloalkylcarbamoyloxy, N—(C₆-C₁₂)-arylcarbamoyloxy, N—(C₇-C₁₆)-aralkylcarbamoyloxy, N—(C₁-C₁₀)-alkyl-N—(C₆-C₁₂)-arylcarbamoyloxy, N(C₁-C₁₀)-alkyl-N—(C₇-C₁₆)-aralkylcarbamoyloxy, N—((C₁-C₁₀)-alkyl)-carbamoyloxy, N—((C₆-C₁₂)-aryloxy-(C₁-C₁₀)-alkyl)-carbamoyloxy, N—((C₇-C₁₆)-aralkyloxy-(C₁-C₁₀)-alkyl)-carbamoyloxy, N—(C₁-C₁₀)-alkyl-N—(C₁-C₁₀)— alkoxy-(C₁-C₁₀)-alkyl)-carbamoyloxy, N—(C₁-C₁₀)-alkyl-N—(C₆-C₁₂)-aryloxy-(C₁-C₁₀)-alkyl)-carbamoyloxy, N—(C₁-C₁₀-alkyl-N—(C₇-C₁₆)-aralkyloxy-(C₁-C₁₀)-alkyl)-carbamoyloxy, amino, (C₁-C₁₂)-alkylamino, di-(C₁-C₁₂)-alkylamino, (C₃-C₈)-cycloalkylamino, (C₃-C₁₂)-alkenylamino, (C₃-C₁₂)-alkynylamino, N—(C₆-C₁₂)-arylamino, N—(C₇-C₁₁)-aralkylamino, N-alkylaralkylamino, N-alkyl-arylamino, (C₁-C₁₂)-alkoxyamino, (C₁-C₁₂)-alkoxy-N—(C₁-C₁₀)-alkylamino, (C₁-C₁₂)-alkylcarbonylamino, (C₃-C₈)-cycloalkylcarbonylamino, (C₆-C₁₂)-arylcarbonylamino, (C₇-C₁₆)-alkylcarbonylamino, (C₁-C₁₂)-alkylcarbonyl-N—(C₁-C₁₀)-alkylamino, (C₃-C₈)-cycloalkylcarbonyl-N—(C₁-C₁₀)-alkylamino, (C₆-C₁₂)-arylcarbonyl-N—(C₁-C₁₀)-alkylamino, (C₇-C₁₁)-aralkylcarbonyl-N—(C₁-C₁₀)-alkylamino, (C₁-C₁₂)-alkylcarbonylamino-(C₁-C₈)-alkyl, (C₃-C₈)-cycloalkylcarbonylamino-(C₁-C₈)-alkyl, (C₆-C₁₂)-arylcarbonylamino-(C₁-C₈)-alkyl, (C₇-C₁₆)-aralkylcarbonylamino-(C₁-C₈)-alkyl, amino-(C₁-C₁₀)-alkyl, N—(C₁-C₁₀)-alkylamino-(C₁-C₁₀)-alkyl, (C₃-C₈)-cycloalkylamino-(C₁-C₁₂)-alkylmercapto, (C₁-C₁₂)-alkylsulfinyl, (C₁-C₁₂)-alkylsulfonyl, (C₆-C₁₂)-arylmercapto, (C₆-C₁₂)-arylsulfinyl, (C₆-C₁₂)-arylsulfonyl, (C₇-C₁₆)-aralkylmercapto, (C₇-C₁₆)-aralkylsulfinyl, or (C₇-C₁₆)-aralkylsulfonyl;

### X is O or S;

Q is O, S, NR′, or a bond;
where, if Q is a bond, R⁴ is halogen, nitrile, or trifluoromethyl;
or where, if Q is O, S, or NR′, R⁴ is hydrogen, (C₁-C₁₀)-alkyl radical, (C₂-C₁₀)-alkenyl radical, (C₂-C₁₀)-alkynyl radical, wherein alkenyl or alkynyl radical contains one or two C—C multiple bonds; unsubstituted fluoroalkyl radical of the formula —[CH₂]ₓC_{f}—H_{(2f+1-g)}—F_{g}, (C₁-C₈)-alkoxy-(C₁-C₆)-alkyl radical, (C₁-C₆)-alkoxy-(C₁-C₄)-alkoxy-(C₁-C₄)-alkyl radical, aryl radical, heteroaryl radical, (C₇-C₁₁)-aralkyl radical, or a radical of the formula Z

—[CH₂]ᵥ—[O]_{w}—[CH₂]ₜ-E (Z)

where
E is a heteroaryl radical, a (C₃-C₈)-cycloalkyl radical, or a phenyl radical of the formula F

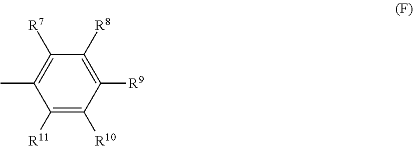

v is 0-6,
w is 0 or 1,
t is 0-3, and
R⁷, R⁸, R⁹, R¹⁰, and R¹¹ are identical or different and are hydrogen, halogen, cyano, nitro, trifluoromethyl, (C₁-C₆)-alkyl, (C₃-C₈)-cycloalkyl, (C₁-C₆)-alkoxy, —O—[CH₂]ₓ—C_{f}H_{(2f+1-g)}—F_{g}, —OCF₂—Cl, —O—CF₂—CHFCl, (C₁-C₆)-alkylmercapto, (C₁-C₆)-hydroxyalkyl, (C₁-C₆)-alkoxy-(C₁-C₆)-alkoxy, (C₁-C₆)-alkoxy-(C₁-C₆)-alkyl, (C₁-C₆)-alkylsulfinyl, (C₁-C₆)-alkylsulfonyl, (C₁-C₆)-alkylcarbonyl, (C₁-C₈)-alkoxycarbonyl, carbamoyl, N—(C₁-C₈)-alkylcarbamoyl, N,N-di-(C₁-C₈)-alkylcarbamoyl, or (C₇-C₁₁)-aralkylcarbamoyl, optionally substituted by fluorine, chlorine, bromine, trifluoromethyl, (C₁-C₆)-alkoxy, N—(C₃-C₈)-cycloalkylcarbamoyl, N—(C₃-C₈)-cycloalkyl-(C₁-C₄)-alkylcarbamoyl, (C₁-C₆)-alkylcarbonyloxy, phenyl, benzyl, phenoxy, benzyloxy, NR^{Y}R^{Z} wherein R^{y} and R^{z} are independently selected from hydrogen, (C₁-C₁₂)-alkyl, (C₁-C₈)-alkoxy-(C₁-C₈)-alkyl, (C₇-C₁₂)-aralkoxy-(C₁-C₈)-alkyl, (C₆-C₁₂)-aryloxy-(C₁-C₈)-alkyl, (C₃-C₁₀)-cycloalkyl, (C₃-C₁₂)-alkenyl, (C₃-C₁₂)-alkynyl, (C₆-C₁₂)-aryl, (C₇-C₁₁)-aralkyl, (C₁-C₁₂)-alkoxy, (C₇-C₁₂)aralkoxy, (C₁-C₁₂)-alkylcarbonyl, (C₃-C₈)-cycloalkylcarbonyl, (C₆-C₁₂) arylcarbonyl, (C₇-C₁₆)-aralkylcarbonyl; or further wherein R^{y} and R^{z} together are —[CH₂]ₕ, in which a CH₂ group can be replaced by O, S, N—(C₁-C₄)-alkylcarbonylimino, or N—(C₁-C₄)-alkoxycarbonylimino; phenylmercapto, phenylsulfonyl, phenylsulfinyl, sulfamoyl, N—(C₁-C₈)-alkylsulfamoyl, or N,N-di-(C₁-C₈)-alkylsulfamoyl; or alternatively R⁷ and R⁸, R⁸ and R⁹, R⁹ and R¹⁰, or R¹⁰ and R¹¹, together are a chain selected from —[CH₂]ₙ— or —CH═CH—CH═CH—, where a CH₂ group of the chain is optionally replaced by O, S, SO, SO₂, or NR^{Y}; and n is 3, 4, or 5; and if E is a heteroaryl radical, said radical can carry 1-3 substituents selected from those defined for R⁷-R¹¹, or if E is a cycloalkyl radical, the radical can carry one substituent selected from those defined for R⁷-R¹¹;
or where, if Q is NR′, R⁴ is alternatively R″, where R′ and R″ are identical or different and are hydrogen, (C₆-C₁₂)-aryl, (C₇-C₁₁)-aralkyl, (C₁-C₈)-alkyl, (C₁-C₈)-alkoxy-(C₁-C₈)-alkyl, (C₇-C₁₂)-aralkoxy-(C₁-C₈)-alkyl, (C₆-C₁₂)-aryloxy-(C₁-C₈)-alkyl, (C₁-C₁₀)-alkylcarbonyl, optionally substituted (C₇-C₁₆)-aralkylcarbonyl, or optionally substituted C₆-C₁₂)-arylcarbonyl; or R′ and R″ together are —[CH₂]ₕ, in which a CH₂ group can be replaced by O, S, N-acylimino, or N—(C₁-C₁₀)-alkoxycarbonylimino, and h is 3 to 7.

### Y is N or CR³;

R¹, R² and R³ are identical or different and are hydrogen, hydroxyl, halogen, cyano, trifluoromethyl, nitro, carboxyl, (C₁-C₂₀)-alkyl, (C₃-C₈)-cycloalkyl, (C₃-C₈)cycloalkyl-(C₁-C₁₂)-alkyl, (C₃-C₈)-cycloalkoxy, (C₃-C₈)-cycloalkyl-(C₁-C₁₂)-alkoxy, (C₃-C₈)-cycloalkyloxy-(C₁-C₁₂)-alkyl, (C₃-C₈)-cycloalkyloxy-(C₁-C₁₂)-alkoxy, (C₃-C₈)-cycloalkyl-(C₁-C₈)-alkyl-(C₁-C₆)-alkoxy, (C₃-C₈)-cycloalkyl-(C₁-C₈)-alkoxy-(C₁-C₆)-alkyl, (C₃-C₈)-cycloalkyloxy-(C₁-C₈)-alkoxy-(C₁-C₆)-alkyl, (C₃-C₈)-cycloalkoxy-(C₁-C₈)-alkoxy-(C₁-C₈)-alkoxy, (C₆-C₁₂)-aryl, (C₇-C₁₆)-aralkyl, (C₇-C₁₆)-aralkenyl, (C₇-C₁₆)-aralkynyl, (C₂-C₂₀)-alkenyl, (C₂-C₂₀)-alkynyl, (C₁-C₂₀)-alkoxy, (C₂-C₂₀)-alkenyloxy, (C₂-C₂₀)-alkynyloxy, retinyloxy, (C₁-C₂₀)-alkoxy-(C₁-C₁₂)-alkyl, (C₁-C₁₂)-alkoxy-(C₁-C₁₂)-alkoxy, (C₁-C₁₂)-alkoxy-(C₁-C₈)-alkoxy-(C₁-C₈)-alkyl, (C₆-C₁₂)-aryloxy, (C₇-C₁₆)-aralkyloxy, (C₆-C₁₂)-aryloxy-(C₁-C₆)-alkoxy, (C₇-C₁₆)-aralkoxy-(C₁-C₆)-alkoxy, (C₁-C₁₆)-hydroxyalkyl, (C₆-C₁₆)-aryloxy-(C₁-C₈)-alkyl, (C₇-C₁₆)-aralkoxy-(C₁-C₈)-alkyl, (C₆-C₁₂)-aryloxy-(C₁-C₈)-alkoxy-(C₁-C₆)-alkyl, (C₇-C₁₂)-aralkyloxy-(C₁-C₈)-alkoxy-(C₁-C₆)-alkyl, (C₂-C₂₀)-alkenyloxy-(C₁-C₆)-alkyl, (C₂-C₂₀)-alkynyloxy-(C₁-C₆)-alkyl, retinyloxy-(C₁-C₆)-alkyl, —O—[CH₂]ₓCfH_{(2f+1-g)}F_{g}, —OCF₂Cl, —OCF₂—CHFCl, (C₁-C₂₀)-alkylcarbonyl, (C₃-C₈)-cycloalkylcarbonyl, (C₆-C₁₂)-arylcarbonyl, (C₇-C₁₆)-aralkylcarbonyl, cinnamoyl, (C₂-C₂₀)-alkenylcarbonyl, (C₂-C₂₀)-alkynylcarbonyl, (C₁-C₂₀)-alkoxycarbonyl, (C₁-C₁₂)-alkoxy-(C₁-C₁₂)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₂₀)-alkenyloxycarbonyl, retinyloxycarbonyl, (C₂-C₂₀)-alkynyloxycarbonyl, (C₆-C₁₂)-aryloxy-(C₁-C₆)-alkoxycarbonyl, (C₇-C₁₆)-aralkoxy-(C₁-C₆)-alkoxycarbonyl, (C₃-C₈)-cycloalkyl-(C₁-C₆)-alkoxycarbonyl, (C₃-C₈)-cycloalkoxy-(C₁-C₆)-alkoxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₁-C₁₂)-alkoxy-(C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N,N-di-(C₁-C₁₂)-alkylcarbamoyl, N—(C₃-C₈)-cycloalkylcarbamoyl, N,N-dicyclo-(C₃-C₈)-alkylcarbamoyl, N—(C₁-C₁₀)-alkyl-N—(C₃-C₈)-cycloalkylcarbamoyl, N—((C₃-C₈)-cycloalkyl-(C₁-C₆)-alkyl)-carbamoyl, N—(C₁-C₆)-alkyl-N—((C₃-C₈)-cycloalkyl-(C₁-C₆)-alkyl)-carbamoyl, N-(+)-dehydroabietylcarbamoyl, N—(C₁-C₆)-alkyl-N-(+)-dehydroabietylcarbamoyl, N—(C₆-C₁₂)-arylcarbamoyl, N—(C₇-C₁₆)-aralkylcarbamoyl, N—(C₁-C₁₀)-alkyl-N—(C₆-C₁₆)-arylcarbamoyl, N—(C₁-C₁₀)-alkyl-N—(C₇-C₁₆)-aralkylcarbamoyl, N—((C₁-C₁₈)-alkoxy-(C₁-C₁₀)-alkyl)-carbamoyl, N—((C₆-C₁₆)-aryloxy-(C₁-C₁₀)-alkyl)-carbamoyl, N—((C₇-C₁₆)-aralkyloxy-(C₁-C₁₀)-alkyl)-carbamoyl, N—(C₁-C₁₀)-alkyl-N—((C₁-C₁₀)-alkoxy-(C₁-C₁₀)-alkyl)-carbamoyl, N—(C₁-C₁₀)-alkyl-N—(C₆-C₁₂)-aryloxy-(C₁-C₁₀)-alkyl)-carbamoyl, N—(C₁-C₁₀)-alkyl-N—((C₇-C₁₆)-aralkyloxy-(C₁-C₁₀)-alkyl)-carbamoyl; CON(CH₂)ₕ, in which a CH₂ group can be replaced by O, S, N—(C₁-C₈)-alkylimino, N—(C₃-C₈)-cycloalkylimino, N—(C₃-C₈)-cycloalkyl-(C₁-C₄)-alkylimino, N—(C₆-C₁₂)-arylimino, N—(C₇-C₁₆)-aralkylimino, N—(C₁-C₄)-alkoxy-(C₁-C₆)-alkylimino, and h is from 3 to 7; a carbamoyl radical of the formula R

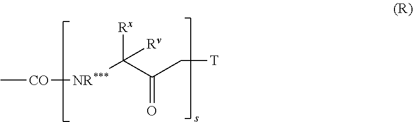

in which
R^{x} and R^{v} are each independently selected from hydrogen, (C₁-C₆)-alkyl, (C₃-C₇)-cycloalkyl, aryl, or the substituent of an α-carbon of an α-amino acid, to which the L- and D-amino acids belong,
s is 1-5,
T is OH, or NR*R**, and R*, R** and R*** are identical or different and are selected from hydrogen, (C₆-C₁₂)-aryl, (C₇-C₁₁)-aralkyl, (C₁-C₈)-alkyl, (C₃-C₈)-cycloalkyl, (+)-dehydroabietyl, (C₁-C₈)-alkoxy-(C₁-C₈)-alkyl, (C₇-C₁₂)-aralkoxy-(C₁-C₈)-alkyl, (C₆-C₁₂)-aryloxy-(C₁-C₈)-alkyl, (C₁-C₁₀)-alkanoyl, optionally substituted (C₇-C₁₆)-aralkanoyl, optionally substituted (C₆-C₁₂)-aroyl; or R* and R** together are —[CH₂]ₕ, in which a CH₂ group can be replaced by O, S, SO, SO₂, N-acylamino, N—(C₁-C₁₀)-alkoxycarbonylimino, N—(C₁-C₈)-alkylimino, N—(C₃-C₈)-cycloalkylimino, N—(C₃-C₈)-cycloalkyl-(C₁-C₄)-alkylimino, N—(C₆-C₁₂)-arylimino, N—(C₇-C₁₆)-aralkylimino, N—(C₁-C₄)-alkoxy-(C₁-C₆)-alkylimino, and h is from 3 to 7; carbamoyloxy, N—(C₁-C₁₂)-alkylcarbamoyloxy, N,N-di-(C₁-C₁₂)-alkylcarbamoyloxy, N—(C₃-C₈)-cycloalkylcarbamoyloxy, N—(C₆-C₁₂)-arylcarbamoyloxy, N—(C₇-C₁₆)-aralkylcarbamoyloxy, N—(C₁-C₁₀)-alkyl-N—(C₆-C₁₂)-arylcarbamoyloxy, N—(C₁-C₁₀)-alkyl-N—(C₇-C₁₆)-aralkylcarbamoyloxy, N—((C₁-C₁₀)-alkyl)-carbamoyloxy, N—((C₆-C₁₂)-aryloxy-(C₁-C₁₀)-alkyl)-carbamoyloxy, N—((C₇-C₁₆)-aralkyloxy-(C₁-C₁₀)-alkyl)-carbamoyloxy, N—(C₁-C₁₀)-alkyl-N—((C₁-C₁₀)-alkoxy-(C₁-C₁₀)-alkyl)-carbamoyloxy, N—(C₁-C₁₀)-alkyl-N—((C₆-C₁₂)-aryloxy-(C₁-C₁₀)-alkyl)-carbamoyloxy, N—(C₁-C₁₀)-alkyl-N—((C₇-C₁₆)-aralkyloxy-(C₁-C₁₀)-alkyl)-carbamoyloxyamino, (C₁-C₁₂)-alkylamino, di-(C₁-C₁₂)-alkylamino, (C₃-C₈)-cycloalkylamino, (C₃-C₁₂)-alkenylamino, (C₃-C₁₂)-alkynylamino, N—(C₆-C₁₂)-arylamino, N—(C₇-C₁₁)-aralkylamino, N-alkyl-aralkylamino, N-alkyl-arylamino, (C₁-C₁₂)-alkoxyamino, (C₁-C₁₂)-alkoxy-N—(C₁-C₁₀)-alkylamino, (C₁-C₁₂)-alkanoylamino, (C₃-C₈)-cycloalkanoylamino, (C₆-C₁₂)-aroylamino, (C₇-C₁₆)-aralkanoylamino, (C₁-C₁₂)-alkanoyl-N—(C₁-C₁₀)-alkylamino, (C₃-C₈)-cycloalkanoyl-N—(C₁-C₁₀)-alkylamino, (C₆-C₁₂)-aroyl-N—(C₁-C₁₀)-alkylamino, (C₇-C₁₁)-aralkanoyl-N—(C₁-C₁₀)-alkylamino, (C₁-C₁₂)-alkanoylamino-(C₁-C₈)-alkyl, (C₃-C₈)-cycloalkanoylamino-(C₁-C₈)-alkyl, (C₆-C₁₂)-aroylamino-(C₁-C₈)-alkyl, (C₇-C₁₆)-aralkanoylamino-(C₁-C₈)-alkyl, amino-(C₁-C₁₀)-alkyl, N—(C₁-C₁₀)-alkylamino-(C₁-C₅₀)-alkyl, N,N-di(C₁-C₁₀)-alkylamino-(C₁-C₁₀)-alkyl, (C₃-C₈)-cycloalkylamino(C₁-C₁₀)-alkyl, (C₁-C₂₀)-alkylmercapto, (C₁-C₂₀)-alkylsulfinyl, (C₁-C₂₀)-alkylsulfonyl, (C₆-C₁₂)-arylmercapto, (C₆-C₁₂)-arylsulfinyl, (C₆-C₁₂)-arylsulfonyl, (C₇-C₁₆)-aralkylmercapto, (C₇-C₁₆)-aralkylsulfinyl, (C₇-C₁₆)-aralkylsulfonyl, (C₁-C₁₂)-alkylmercapto-(C₁-C₆)-alkyl, (C₁-C₁₂)-alkylsulfinyl-(C₁-C₆)-alkyl, (C₁-C₁₂)-alkylsulfonyl-(C₁-C₆)-alkyl, (C₆-C₁₂)-arylmercapto-(C₁-C₆)-alkyl, (C₆-C₁₂)-arylsulfinyl-(C₁-C₆)-alkyl, (C₆-C₁₂)-arylsulfonyl-(C₁-C₆)-alkyl, (C₇-C₁₆)-aralkylmercapto-(C₁-C₆)-alkyl, (C₇-C₁₆)-aralkylsulfinyl-(C₁-C₆)-alkyl, (C₇-C₁₆)-aralkylsulfonyl-(C₁-C₆)-alkyl, sulfamoyl, N—(C₁-C₁₀)-alkylsulfamoyl, N,N-di-(C₁-C₅₀)-alkylsulfamoyl, (C₃-C₈)-cycloalkylsulfamoyl, N—(C₆-C₁₂)-arylsulfamoyl, N—(C₇-C₁₆)-aralkylsulfamoyl, N—(C₁-C₁₀)-alkyl-N—(C₆-C₁₂)-arylsulfamoyl, N—(C₁-C₁₀)-alkyl-N—(C₇-C₁₆)-aralkylsulfamoyl, (C₁-C₁₀)-alkylsulfonamido, N—((C₁-C₁₀)-alkyl)-(C₁-C₁₀)-alkylsulfonamido, (C₇-C₁₆)-aralkylsulfonamido, and N—((C₁-C₁₀)-alkyl-(C₇-C₁₆)-aralkylsulfonamido; where an aryl radical may be substituted by 1 to 5 substituents selected from hydroxyl, halogen, cyano, trifluoromethyl, nitro, carboxyl, (C₂-C₁₆)-alkyl, (C₃-C₈)-cycloalkyl, (C₃-C₈)-cycloalkyl-(C₁-C₁₂)-alkyl, (C₃-C₈)-cycloalkoxy, (C₃-C₈)-cycloalkyl-(C₁-C₁₂)-alkoxy, (C₃-C₈)-cycloalkyloxy-(C₁-C₁₂)-alkyl, (C₃-C₈)-cycloalkyloxy-(C₁-C₁₂)-alkoxy, (C₃-C₈)-cycloalkyl-(C₁-C₈)-alkyl-(C₁-C₆)-alkoxy, (C₃-C₈)-cycloalkyl(C₁-C₈)-alkoxy-(C₁-C₆)-alkyl, (C₃-C₈)-cycloalkyloxy-(C₁-C₈)-alkoxy-(C₁-C₆)-alkyl, (C₃-C₈)-cycloalkoxy-(C₁-C₈)-alkoxy-(C₁-C₈)-alkoxy, (C₆-C₁₂)-aryl, (C₇-C₁₆)-aralkyl, (C₂-C₁₆)-alkenyl, (C₂-C₁₂)-alkynyl, (C₁-C₁₆)-alkoxy, (C₁-C₁₆)-alkenyloxy, (C₁-C₁₂)-alkoxy-(C₁-C₁₂)-alkyl, (C₁-C₁₂)-alkoxy-(C₁-C₁₂)-alkoxy, (C₁-C₁₂)-alkoxy(C₁-C₈)-alkoxy-(C₁-C₈)-alkyl, (C₆-C₁₂)-aryloxy, (C₇-C₁₆)-aralkyloxy, (C₆-C₁₂)-aryloxy-(C₁-C₆)-alkoxy, (C₇-C₁₆)-aralkoxy-(C₁-C₆)-alkoxy, (C₁-C₈)-hydroxyalkyl, (C₆-C₁₆)-aryloxy-(C₁-C₈)-alkyl, (C₇-C₁₆)-aralkoxy-(C₁-C₈)-alkyl, (C₆-C₁₂)-aryloxy-(C₁-C₈)-alkoxy-(C₁-C₆)-alkyl, (C₇-C₁₂)-aralkyloxy-(C₁-C₈)-alkoxy-(C₁-C₆)-alkyl, —O—[CH₂]ₓ—C_{f}H_{(2f+1-g)}F_{g}, —OCF₂Cl, —OCF₂—CHFCl, (C₁-C₁₂)-alkylcarbonyl, (C₃-C₈)-cycloalkylcarbonyl, (C₆-C₁₂)-arylcarbonyl, (C₇-C₁₆)-aralkylcarbonyl, (C₁-C₁₂)-alkoxycarbonyl, (C₁-C₁₂)-alkoxy-(C₁-C₁₂)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₁₂)-alkenyloxycarbonyl, (C₂-C₁₂)-alkynyloxycarbonyl, (C₆-C₁₂)-aryloxy-(C₁-C₆)-alkoxycarbonyl, (C₇-C₁₆)-aralkoxy-(C₁-C₆)-alkoxycarbonyl, (C₃-C₈)-cycloalkyl-(C₁-C₆)-alkoxycarbonyl, (C₃-C₈)-cycloalkoxy-(C₁-C₆)-alkoxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₁-C₁₂)-alkoxy-(C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N,N-di(C₁-C₁₂)-alkylcarbamoyl, N—(C₃-C₈)-cycloalkylcarbamoyl, N,N-dicyclo-(C₃-C₈)-alkylcarbamoyl, N—(C₁-C₁₀)-alkyl-N—(C₃-C₈)-cycloalkylcarbamoyl, N—((C₃-C₈)-cycloalkyl-(C₁-C₆)-alkyl)carbamoyl, N—(C₁-C₆)-alkyl-N—((C₃-C₈)-cycloalkyl-(C₁-C₆)-alkyl)carbamoyl, N-(+)-dehydroabietylcarbamoyl, N—(C₁-C₆)-alkyl-N-(+)-dehydroabietylcarbamoyl, N—(C₆-C₁₂)-arylcarbamoyl, N—(C₇-C₁₆)-aralkylcarbamoyl, N—(C₁-C₁₀)-alkyl-N—(C₆-C₁₆)-arylcarbamoyl, N—(C₁-C₁₀)-alkyl-N—(C₇-C₁₆)-aralkylcarbamoyl, N—((C₁-C₁₆)-alkoxy-(C₁-C₁₀)-alkyl)carbamoyl, N—((C₆-C₁₆)-aryloxy-(C₁-C₁₀)-alkyl)carbamoyl, N—((C₇-C₁₆)-aralkyloxy-(C alkyl)carbamoyl, N—(C₁-C₁₀)-alkyl-N—((C₁-C₁₀)-alkoxy-(C₁-C₁₀)-alkyl)carbamoyl, N—(C₁-C₁₀)-alkyl-N—((C₆-C₁₂)-aryloxy-(C₁-C₁₀)-alkyl)carbamoyl, N—(C₁-C₁₀)-alkyl-N—((C₇-C₁₆)-aralkyloxy-(C₁-C₁₀)-alkyl)-carbamoyl, CON(CH₂)ₕ, in which a CH₂ group can be replaced by, O, S, N—(C₁-C₈)-alkylimino, N—(C₃-C₈)-cycloalkylimino, N—(C₃-C₈)-cycloalkyl-(C₁-C₄)-alkylimino, N—(C₆-C₁₂)-arylimino, N—(C₇-C₁₆)-aralkylimino, N—(C₁-C₄)-alkoxy-(C₁-C₆)-alkylimino, and h is from 3 to 7; carbamoyloxy, N—(C₁-C₁₂)-alkylcarbamoyloxy, N,N-di-(C₁-C₁₂)-alkylcarbamoyloxy, N—(C₃-C₈)-cycloalkylcarbamoyloxy, N—(C₆-C₁₆)-arylcarbamoyloxy, N—(C₇-C₁₆)-aralkylcarbamoyloxy, N—(C₁-C₁₀)-alkyl-N—(C₆-C₁₂)-arylcarbamoyloxy, N—(C₁-C₁₀)-alkyl-N—(C₇-C₁₆)-aralkylcarbamoyloxy, N—((C₁-C₁₀)-alkyl)carbamoyloxy, N—((C₆-C₁₂)-aryloxy-(C₁-C₁₀)-alkyl)carbamoyloxy, N—((C₇-C₁₆)-aralkyloxy-(C₁-C₁₀)-alkyl)carbamoyloxy, N—(C₁-C₁₀)-alkyl-N—((C₁-C₁₀)-alkoxy-(C₁-C₁₀)-alkyl)carbamoyloxy, N—(C₁-C₁₀)-alkyl-N—((C₆-C₁₂)-aryloxy-(C₁-C₁₀)-alkyl)carbamoyloxy, N—(C₁-C₁₀)-alkyl-N—((C₇-C₁₆)-aralkyloxy-(C₁-C₁₀)-alkyl)carbamoyloxy, amino, (C₁-C₁₂)-alkylamino, di-(C₁-C₁₂)-alkylamino, (C₃-C₈)-cycloalkylamino, (C₃-C₁₂)-alkenylamino, (C₃-C₁₂)-alkynylamino, N—(C₆-C₁₂)-arylamino, N—(C₇-C₁₁)-aralkylamino, N-alkyl-aralkylamino, N-alkyl-arylamino, (C₁-C₁₂)-alkoxyamino, (C₁-C₁₂)-alkoxy-N—(C₁-C₁₀)-alkylamino, (C₁-C₁₂)-alkanoylamino, (C₃-C₈)-cycloalkanoylamino, (C₆-C₁₂)-aroylamino, (C₇-C₁₆)-aralkanoylamino, (C₁-C₁₂)-alkanoyl-N—(C₁-C₁₀)-alkylamino, (C₃-C₈)-cycloalkanoyl-N—(C₁-C₁₀)-alkylamino, (C₆-C₁₂)-aroyl-N—(C₁-C₁₀)-alkylamino, (C₇-C₁₁)-aralkanoyl-N—(C₁-C₁₀)-alkylamino, (C₁-C₁₂)-alkanoylamino-(C₁-C₈)-alkyl, (C₃-C₈)-cycloalkanoylamino-(C₁-C₈)-alkyl, (C₆-C₁₂)-aroylamino-(C₁-C₈)-alkyl, (C₇-C₁₆)-aralkanoylamino-(C₁-C₈)-alkyl, amino-(C₁-C₁₀)-alkyl, N—(C₁-C₁₀)-alkylamino-(C₁-C₁₀)-alkyl, N,N-di-(C₁-C₁₀)-alkylamino-(C₁-C₁₀)-alkyl, (C₃-C₈)-cycloalkylamino-(C₁-C₁₀)-alkyl, (C₁-C₁₂)-alkylmercapto, (C₁-C₁₂)-alkylsulfinyl, (C₁-C₁₂)-alkylsulfonyl, (C₆-C₁₆)-arylmercapto, (C₆-C₁₆)-arylsulfinyl, (C₆-C₁₆)-arylsulfonyl, (C₇-C₁₆)-aralkylmercapto, (C₇-C₁₆)-aralkylsulfinyl, or (C₇-C₁₆)-aralkylsulfonyl;
or wherein R¹ and R², or R² and R³ form a chain [CH₂]ₒ, which is saturated or unsaturated by a C═C double bond, in which 1 or 2 CH₂ groups are optionally replaced by O, S, SO, SO₂, or NR′, and R′ is hydrogen, (C₆-C₁₂)-aryl, (C₁-C₈)-alkyl, (C₁-C₈)-alkoxy-(C₁-C₈)-alkyl, (C₇-C₁₂)-aralkoxy-(C₁-C₈)-alkyl, (C₆-C₁₂)-aryloxy-(C₁-C₈)-alkyl, (C₁-C₁₀)-alkanoyl, optionally substituted (C₇-C₁₆)-aralkanoyl, or optionally substituted (C6-C12)-aroyl; and o is 3, 4 or 5;
or wherein the radicals R¹ and R², or R² and R³, together with the pyridine or pyridazine carrying them, form a 5,6,7,8-tetrahydroisoquinoline ring, a 5,6,7,8-tetrahydroquinoline ring, or a 5,6,7,8-tetrahydrocinnoline ring;
or wherein R¹ and R², or R² and R³ form a carbocyclic or heterocyclic 5- or 6-membered aromatic ring;
or where R¹ and R², or R² and R³, together with the pyridine or pyridazine carrying them, form an optionally substituted heterocyclic ring systems selected from thienopyridines, furanopyridines, pyridopyridines, pyrimidinopyridines, imidazopyridines, thiazolopyridines, oxazolopyridines, quinoline, isoquinoline, and cinnoline; where quinoline, isoquinoline or cinnoline preferably satisfy the formulae Ia, Ib and Ic:

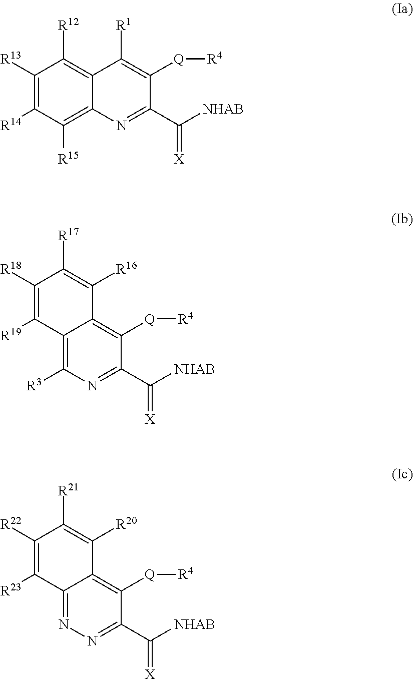

and the substituents R¹² to R²³ in each case independently of each other have the meaning of R¹, R² and R³;
or wherein the radicals R¹ and R², together with the pyridine carrying them, form a compound of Formula Id:

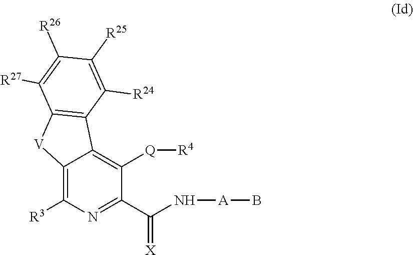

where V is S, O, or NR^{k}, and R^{k} is selected from hydrogen, (C₁-C₆)-alkyl, aryl, or benzyl; where an aryl radical may be optionally substituted by 1 to 5 substituents as defined above; and
R²⁴, R²⁵, R²⁶, and R²⁷ in each case independently of each other have the meaning of R¹, R² and R³;
f is 1 to 8;
g is 0 or 1 to (2f+1);
x is 0 to 3; and
h is 3 to 7;
including the physiologically active salts and prodrugs derived therefrom.

Compounds of Formulae (I), (Ia), (Ib), (Ic), and (Id) are representative of the heterocyclic carboxamides identified, supra, as being suitable for use in the present invention. Exemplary compounds according to Formula (I) are described in European Patent Nos. EP0650960 and EP0650961. All compounds listed in EP0650960 and EP0650961, in particular, those listed in the compound claims and the final products of the working examples, are hereby incorporated into the present application by reference herein.

Additionally, exemplary compounds according to Formula (I) are described in U.S. Pat. No. 5,658,933. All compounds listed in U.S. Pat. No. 5,658,933, in particular, those listed in the compound claims and the final products of the working examples, are hereby incorporated into the present application by reference herein.

Additional compounds according to Formula (I) are substituted heterocyclic carboxyamides described in U.S. Pat. No. 5,620,995; 3-hydroxypyridine-2-carboxamidoesters described in U.S. Pat. No. 6,020,350; sulfonamidocarbonylpyridine-2-carboxamides described in U.S. Pat. No. 5,607,954; and sulfonamidocarbonyl-pyridine-2-carboxamides and sulfonamidocarbonyl-pyridine-2-carboxamide esters described in U.S. Pat. Nos. 5,610,172 and 5,620,996. All compounds listed in these patents, in particular, those compounds listed in the compound claims and the final products of the working examples, are hereby incorporated into the present application by reference herein.

Exemplary compounds according to Formula (Ia) are described in U.S. Pat. Nos. 5,719,164 and 5,726,305. All compounds listed in the foregoing patents, in particular, those listed in the compound claims and the final products of the working examples, are hereby incorporated into the present application by reference herein.

As discussed, supra, compounds according the present invention are in some embodiments heterocyclic carboxamides; in particular, quinoline carboxamides. In certain embodiments, compounds for use in the invention are quinoline-2-carboxamides. In one embodiment, the compound is selected from a compound of the Formula Ia wherein
A is —CR⁵R⁶—, and R⁵ and R⁶ are each independently selected from the group consisting of hydrogen, (C₁-C₆)-alkyl, (C₃-C₇)-cycloalkyl, aryl, or a substituent of the α-carbon atom of an α-amino acid, wherein the amino acid is a natural L-amino acid or its D-isomer;
B is —CO₂H or a CO₂-G carboxyl radical, where G is a radical of an alcohol G-OH in which G is selected from the group consisting of (C₁-C₂₀)-alkyl radical, (C₃-C₈) cycloalkyl radical, (C₂-C₂₀)-alkenyl radical, (C₃-C₈)-cycloalkenyl radical, retinyl radical, (C₂-C₂₀)-alkynyl radical, (C₄-C₂₀)-alkenynyl radical;
X is O;
Q is O;
R⁴ is selected from the group consisting of hydrogen, (C₁-C₁₀)-alkyl, (C₂-C₁₀)-alkenyl, (C₂-C₁₀)-alkynyl, wherein alkenyl or alkynyl contains one or two C—C multiple bonds; unsubstituted fluoroalkyl radical of the formula —[CH₂]ₓ—C_{f}H_{(2f+1-g)} —F_{g}, aryl, heteroaryl, and (C₇-C₁₁)-aralkyl;
R¹, R¹², R¹³, R¹⁴ and R¹⁵ are identical or different and are selected from the group consisting of hydrogen, hydroxyl, halogen, cyano, trifluoromethyl, nitro, carboxyl; (C₁-C₂₀)-alkyl, (C₃-C₈)-cycloalkyl, (C₃-C₈)-cycloalkoxy, (C₆-C₁₂)-aryl, (C₇-C₁₆)-aralkyl, (C₇-C₁₆)-aralkenyl, (C₇-C₁₆)-aralkynyl, (C₂-C₂₀)-alkenyl, (C₂-C₂₀)-alkynyl, (C₁-C₂₀)-alkoxy, (C₂-C₂₀)-alkenyloxy, (C₂-C₂₀)-alkynyloxy, retinyloxy, (C₆-C₁₂)-aryloxy, (C₇-C₁₆)-aralkyloxy, (C₁-C₁₆)-hydroxyalkyl, —O—[CH₂]ₓCfH_{(2f+1-g)}F_{g}, —OCF₂Cl, —OCF₂—CHFCl, (C₁-C₂₀)-alkylcarbonyl, (C₃-C₃)-cycloalkylcarbonyl, (C₆-C₁₂)-arylcarbonyl, (C₇-C₁₆)-aralkylcarbonyl, cinnamoyl, (C₂-C₂₀)-alkenylcarbonyl, (C₂-C₂₀)-alkynylcarbonyl, (C₁-C₂₀)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₂₀)-alkenyloxycarbonyl, retinyloxycarbonyl, (C₂-C₂₀)-alkynyloxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N,N-di-(C₁-C₁₂)-alkylcarbamoyl, N—(C₃-C₈)-cycloalkylcarbamoyl, N,N-dicyclo-(C₃-C₈)-alkylcarbamoyl, N—(C₁-C₁₀)-alkyl-N—(C₃-C₈)-cycloalkylcarbamoyl, N—((C₃-C₈)-cycloalkyl-(C₁-C₆)-alkyl)-carbamoyl, N-(+)-dehydroabietylcarbamoyl, N—(C₁-C₆)-alkyl-N-(+)-dehydroabietylcarbamoyl, N—(C₆-C₁₂)-arylcarbamoyl, N—(C₇-C₁₆)-aralkylcarbamoyl, N—(C₁-C₁₀)-alkyl-N—(C₆-C₁₆)-arylcarbamoyl, N—(C₁-C₁₀)-alkyl-N—(C₇-C₁₆)-aralkylcarbamoyl, carbamoyloxy, N—(C₁-C₁₂)-alkylcarbamoyloxy, N,N-di-(C₁-C₁₂)-alkylcarbamoyloxy, N—(C₃-C₈)-cycloalkylcarbamoyloxy, N—(C₆-C₁₂)-arylcarbamoyloxy, N—(C₇-C₁₆)-aralkylcarbamoyloxy, N—(C₁-C₁₀)-alkyl-N—(C₆-C₁₂)-arylcarbamoyloxy, N—(C₁-C₁₀)-alkyl-N—(C₇-C₁₆)-aralkylcarbamoyloxy, N—((C₁-C₁₀)-alkyl)-carbamoyloxy, N—(C₁-C₁₀)-alkyl-N—((C₇-C₁₆)-aralkyloxy-(C₁-C₁₀)-alkyl)-carbamoyloxyamino, (C₁-C₁₂)-alkylamino, di-(C₁-C₁₂)-alkylamino, (C₃-C₈)-cycloalkylamino, (C₃-C₁₂)-alkenylamino, (C₃-C₁₂)-alkynylamino, N—(C₆-C₁₂)-arylamino, N—(C₇-C₁₁)-aralkylamino, N-alkyl-aralkylamino, N-alkyl-arylamino, (C₁-C₁₂)-alkoxyamino, (C₁-C₁₂)-alkoxy-N—(C₁-C₁₀)-alkylamino, (C₁-C₁₂)-alkanoylamino, (C₃-C₈)-cycloalkanoylamino, (C₆-C₁₂)-aroylamino, (C₇-C₁₆)-aralkanoylamino, (C₁-C₁₂)-alkanoyl-N—(C₁-C₁₀)-alkylamino, (C₃-C₈)-cycloalkanoyl-N—(C₁-C₁₀)-alkylamino, (C₆-C₁₂)-aroyl-N—(C₁-C₁₀)-alkylamino, (C₇-C₁₁)-aralkanoyl-N—(C₁-C₁₀)-alkylamino, amino-(C₁-C₁₀)-alkyl, (C₁-C₂₀)-alkylmercapto, (C₁-C₂₀)-alkylsulfinyl, (C₁-C₂₀-alkylsulfonyl, (C₆-C₁₂)-arylmercapto, (C₆-C₁₂)-arylsulfinyl, (C₆-C₁₂)-arylsulfonyl, (C₇-C₁₆)-aralkylmercapto, (C₇-C₁₆)-aralkylsulfinyl, (C₇-C₁₆)-aralkylsulfonyl, sulfamoyl, N—(C₁-C₁₀)-alkylsulfamoyl, N,N-di-(C₁-C₁₀)-alkylsulfamoyl, (C₃-C₈)-cycloalkylsulfamoyl, N—(C₆-C₁₂)-arylsulfamoyl, N—(C₇-C₁₆)-aralkylsulfamoyl, N—(C₁-C₁₀)-alkyl-N—(C₆-C₁₂)-arylsulfamoyl, N—(C₁-C₁₀)-alkyl-N—(C₇-C₁₆)-aralkylsulfamoyl, (C₁-C₁₀)-alkylsulfonamido, (C₇-C₁₆)-aralkylsulfonamido, and N—((C₁-C₁₀)-alkyl-(C₇-C₁₆)-aralkylsulfonamido; where an aryl radical may be substituted by 1 to 5 substituents selected from hydroxyl, halogen, cyano, trifluoromethyl, nitro, carboxyl, (C₂-C₁₆)-alkyl, (C₃-C₈)-cycloalkyl, (C₃-C₈)-cycloalkoxy, (C₆-C₁₂)-aryl, (C₇-C₁₆)-aralkyl, (C₂-C₁₆)-alkenyl, (C₂-C₁₂)-alkynyl, (C₁-C₁₆)-alkoxy, (C₁-C₁₆)-alkenyloxy, (C₆-C₁₂)-aryloxy, (C₇-C₁₆)-aralkyloxy, (C₁-C₈)-hydroxyalkyl, —O—[CH₂]ₓ—C_{f}H_{(2f+1-g)}F_{g}, —OCF₂Cl, and —OCF₂—CHFCl;
x is 0 to 3;
f is 1 to 8; and
g is 0 or 1 to (2f+1);
including the physiologically active salts, esters, and prodrugs derived therefrom.

In certain embodiments, the quinoline-2-carboxamide is selected from a compound of the Formula Ia wherein
A is —CHR⁵ and R⁵ is hydrogen or methyl;
B is —CO₂H;
X is O;
Q is O;
R⁴ is hydrogen; and
R¹, R¹², R¹³, R¹⁴ and R¹⁵ are identical or different and are selected from the group consisting hydrogen, chloro, aryl, aryloxy, and substituted aryloxy,
including the physiologically active salts, esters, and prodrugs derived therefrom.

Exemplary compounds according to Formula (Ib) are described in U.S. Pat. No. 6,093,730. All compounds listed in U.S. Pat. No. 6,093,730, in particular, those listed in the compound claims and the final products of the working examples, are hereby incorporated into the present application by reference herein.

As discussed previously, compounds according to the present invention include isoquinoline carboxamides. In certain embodiments, compounds for use in the invention are isoquinoline-3-carboxamides. In one embodiment, the isoquinoline-3-carboxamide is selected from a compound of the Formula Ib wherein
A is —CR⁵R⁶—, and R⁵ and R⁶ are each independently selected from the group consisting of hydrogen, (C₁-C₆)-alkyl, (C₃-C₇)-cycloalkyl, aryl, or a substituent of the α-carbon atom of an α-amino acid, wherein the amino acid is a natural L-amino acid or its D-isomer;
B is —CO₂H or a CO₂-G carboxyl radical, where G is a radical of an alcohol G-OH in which G is selected from the group consisting of (C₁-C₂₀)-alkyl radical, (C₃-C₈) cycloalkyl radical, (C₂-C₂₀)-alkenyl radical, (C₃-C₈)-cycloalkenyl radical, retinyl radical, (C₂-C₂₀)-alkynyl radical, (C₄-C₂₀)-alkenynyl radical;
X is O;
Q is O;
R⁴ is selected from the group consisting of hydrogen, (C₁-C₁₀)-alkyl, (C₂-C₁₀)-alkenyl, (C₂-C₁₀)-alkynyl, wherein alkenyl or alkynyl contains one or two C—C multiple bonds; unsubstituted fluoroalkyl radical of the formula —[CH₂]ₓ—C_{f}H_{(2f+1-g)}—F_{g}, aryl, heteroaryl, and (C₇-C₁₁)-aralkyl;
R³, R¹⁶, R¹⁷, R¹⁸ and R¹⁹ are identical or different and are selected from the group consisting of hydrogen, hydroxyl, halogen, cyano, trifluoromethyl, nitro, carboxyl; (C₁-C₂₀)-alkyl, (C₃-C₈)-cycloalkyl, (C₃-C₈)-cycloalkoxy, (C₆-C₁₂)-aryl, (C₇-C₁₆)-aralkyl, (C₇-C₁₆)-aralkenyl, (C₇-C₁₆)-aralkynyl, (C₂-C₂₀)-alkenyl, (C₂-C₂₀)-alkynyl, (C₁-C₂₀)-alkoxy, (C₂-C₂₀)-alkenyloxy, (C₂-C₂₀)-alkynyloxy, retinyloxy, (C₆-C₁₂)-aryloxy, (C₇-C₁₆)-aralkyloxy, (C₁-C₁₆)-hydroxyalkyl, —O—[CH₂]ₓCfH_{(2f+1-g)}F_{g}, —OCF₂Cl, —OCF₂—CHFCl, (C₁-C₂₀)-alkylcarbonyl, (C₃-C₈)-cycloalkylcarbonyl, (C₆-C₁₂)-arylcarbonyl, (C₇-C₁₆)-aralkylcarbonyl, cinnamoyl, (C₂-C₂₀)-alkenylcarbonyl, (C₂-C₂₀)-alkynylcarbonyl, (C₁-C₂₀)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₂₀)-alkenyloxycarbonyl, retinyloxycarbonyl, (C₂-C₂₀)-alkynyloxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N,N-di-(C₁-C₁₂)-alkylcarbamoyl, N—(C₃-C₈)-cycloalkylcarbamoyl, N,N-dicyclo-(C₃-C₈)-alkylcarbamoyl, N—(C₁-C₁₀)-alkyl-N—(C₃-C₈)-cycloalkylcarbamoyl, N—(C₃-C₈)-cycloalkyl-(C₁-C₆)-alkyl)-carbamoyl, N-(+)-dehydroabietylcarbamoyl, N—(C₁-C₆)-alkyl-N-(+)-dehydroabietylcarbamoyl, N—(C₆-C₁₂)-arylcarbamoyl, N—(C₇-C₁₆)-aralkylcarbamoyl, N—(C₁-C₁₀)-alkyl-N—(C₆-C₁₆)-arylcarbamoyl, N—(C₁-C₁₀)-alkyl-N—(C₇-C₁₆)-aralkylcarbamoyl, carbamoyloxy, N—(C₁-C₁₂)-alkylcarbamoyloxy, N,N-di-(C₁-C₁₂)-alkylcarbamoyloxy, N—(C₃-C₈)-cycloalkylcarbamoyloxy, N—(C₆-C₁₂)-arylcarbamoyloxy, N—(C₇-C₁₆)-aralkylcarbamoyloxy, N—(C₁-C₁₀)-alkyl-N—(C₆-C₁₂)-arylcarbamoyloxy, N—(C₁-C₁₀)-alkyl-N—(C₇-C₁₆)-aralkylcarbamoyloxy, N—((C₁-C₁₀)-alkyl)-carbamoyloxy, N—(C₁-C₁₀)-alkyl-N—(C₇-C₁₆)-aralkyloxy-(C₁-C₁₀)-alkyl)-carbamoyloxyamino, (C₁-C₁₂)-alkylamino, di-(C₁-C₁₂)-alkylamino, (C₃-C₈)-cycloalkylamino, (C₃-C₁₂)-alkenylamino, (C₃-C₁₂)-alkynylamino, N—(C₆-C₁₂)-arylamino, N—(C₇-C₁₁)-aralkylamino, N-alkyl-aralkylamino, N-alkyl-arylamino, (C₁-C₁₂)-alkoxyamino, (C₁-C₁₂)-alkoxy-N—(C₁-C₁₀)-alkylamino, (C₁-C₁₂)-alkanoylamino, (C₃-C₈)-cycloalkanoylamino, (C₆-C₁₂)-aroylamino, (C₇-C₁₆)-aralkanoylamino, (C₁-C₁₂)-alkanoyl-N—(C₁-C₁₀)-alkylamino, (C₃-C₈)-cycloalkanoyl-N—(C₁-C₁₀)-alkylamino, (C₆-C₁₂)-aroyl-N—(C₁-C₁₀)-alkylamino, (C₇-C₁₁)-aralkanoyl-N—(C₁-C₁₀)-alkylamino, amino-(C₁-C₁₀)-alkyl, (C₁-C₂₀)-alkylmercapto, (C₁-C₂₀)-alkylsulfinyl, (C₁-C₂₀)-alkylsulfonyl, (C₆-C₁₂)-arylmercapto, (C₆-C₁₂)-arylsulfinyl, (C₆-C₁₂)-arylsulfonyl, (C₇-C₁₆)-aralkylmercapto, (C₇-C₁₆)-aralkylsulfinyl, (C₇-C₁₆)-aralkylsulfonyl, sulfamoyl, N—(C₁-C₁₀)-alkylsulfamoyl, N,N-di-(C₁-C₁₀)-alkylsulfamoyl, (C₃-C₈)-cycloalkylsulfamoyl, N—(C₆-C₁₂)-arylsulfamoyl, N—(C₇-C₁₆)-aralkylsulfamoyl, N—(C₁-C₁₀)-alkyl-N—(C₆-C₁₂)-arylsulfamoyl, N—(C₁-C₁₀)-alkyl-N—(C₇-C₁₆)-aralkylsulfamoyl, (C₁-C₁₀)-alkylsulfonamido, (C₇-C₁₆)-aralkylsulfonamido, and N—(C₁-C₁₀)-alkyl-(C₇-C₁₆)-aralkylsulfonamido; where an aryl radical may be substituted by 1 to 5 substituents selected from hydroxyl, halogen, cyano, trifluoromethyl, nitro, carboxyl, (C₂-C₁₆)-alkyl, (C₃-C₈)-cycloalkyl, (C₃-C₈)-cycloalkoxy, (C₆-C₁₂)-aryl, (C₇-C₁₆)-aralkyl, (C₂-C₁₆)-alkenyl, (C₂-C₁₂)-alkynyl, (C₁-C₁₆)-alkoxy, (C₁-C₁₆)-alkenyloxy, (C₆-C₁₂)-aryloxy, (C₇-C₁₆)-aralkyloxy, (C₁-C₈)-hydroxyalkyl, —O—[CH₂]ₓ—C_{f}H_{(2f+1-g)}F_{g}, —OCF₂Cl, and —OCF₂—CHFCl;
x is 0 to 3;
f is 1 to 8; and
g is 0 or 1 to (2f+1);
including the physiologically active salts, esters, and prodrugs derived therefrom.

In one particular embodiment therein, the isoquinoline-3-carboxamide is selected from a compound of the Formula Ib wherein
A is —CHR⁵ where R⁵ is selected hydrogen or methyl; Bis —CO₂H;
X is O;
Q is O;
R⁴ is hydrogen, (C₁-C₃)-alkyl, or substituted (C₁-C₃)-alkyl;
R³ is hydrogen, chloro, or cyano; and
R¹⁶, R¹⁷, R¹⁸ and R¹⁹ are independently selected from the group consisting of hydrogen, halo, alkyl, substituted alkyl, aryl, heteroaryl, substituted heteroaryl, —OR⁷⁰, —SR⁷⁰, —SOR⁷⁰, and —SO₂R⁷⁰ wherein R⁷⁰ is selected from the group consisting of alkyl, substituted alkyl, cylcoalkyl, substituted cycloalkyl, aryl, substituted aryl, heteroaryl, and substituted heteroaryl;
including the physiologically active salts, esters, and prodrugs derived therefrom.

In other embodiments, isoquinoline-3-carboxamides for use in the present invention include those disclosed in International Publication No. WO 2004/108681 and as represented by Formula IV, IVA, IVB, IVC, IVD, VA, VB, VC and VD below.

As discussed, supra, compounds according the present invention are in some embodiments heterocyclic carboxamides. In certain embodiments, heterocyclic carboxamides for use in the invention may be thienopyridine carboxamides. In particular embodiments, the thienopyridine carboxamide is selected from a thienopyridine-5-carboxamide or a thienopyridine-6-carboxamide. In another embodiment, thienopyridine carboxamide compounds for use in the present invention are as disclosed in International Publication No. WO 2006/094292, represented by Formula II

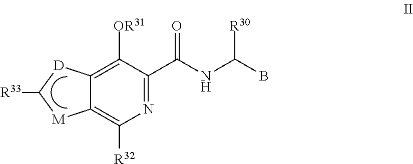

wherein
R³⁰ is selected from the group consisting of hydrogen, (C₁-C₆)-alkyl, (C₃-C₇)-cycloalkyl, aryl, or a substituent of the α-carbon atom of an α-amino acid, wherein the amino acid is a natural L-amino acid or its D-isomer;
B is —CO₂H or a CO₂-G carboxyl radical, where G is a radical of an alcohol G-OH in which G is selected from the group consisting of (C₁-C₂₀)-alkyl radical, (C₃-C₈) cycloalkyl radical, (C₂-C₂₀)-alkenyl radical, (C₃-C₈)-cycloalkenyl radical, retinyl radical, (C₂-C₂₀)-alkynyl radical, (C₄-C₂₀)-alkenynyl radical;
R³¹ is selected from the group consisting of hydrogen, (C₁-C₁₀)-alkyl, (C₂-C₁₀)-alkenyl, (C₂-C₁₀)-alkynyl, wherein alkenyl or alkynyl contains one or two C—C multiple bonds; unsubstituted fluoroalkyl radical of the formula —[CH₂]ₓ—C_{f}H_{(2f+1-g)}F_{g}, aryl, heteroaryl, and (C₇-C₁₁)-aralkyl; one of D or M is —S—, and the other is ═C(R³⁴)—;
R³², R³³, and R³⁴ are identical or different and are selected from the group consisting of hydrogen, hydroxyl, halogen, cyano, trifluoromethyl, nitro, carboxyl; (C₁-C₂₀)-alkyl, (C₃-C₈)-cycloalkyl, (C₃-C₈)-cycloalkoxy, (C₆-C₁₂)-aryl, (C₇-C₁₆)-aralkyl, (C₇-C₁₆)-aralkenyl, (C₇-C₁₆)-aralkynyl, (C₂-C₂₀)-alkenyl, (C₂-C₂₀)-alkynyl, (C₁-C₂₀)-alkoxy, (C₂-C₂₀)-alkenyloxy, (C₂-C₂₀)-alkynyloxy, retinyloxy, (C₆-C₁₂)-aryloxy, (C₇-C₁₆)-aralkyloxy, (C₁-C₁₆)-hydroxyalkyl, —O—[CH₂]ₓCfH_{(2f+1-g)}F_{g}, —OCF₂Cl, —OCF₂—CHFCl, (C₁-C₂₀)-alkylcarbonyl, (C₃-C₈)-cycloalkylcarbonyl, (C₆-C₁₂)-arylcarbonyl, (C₇-C₁₆)-aralkylcarbonyl, cinnamoyl, (C₂-C₂₀)-alkenylcarbonyl, (C₂-C₂₀)-alkynylcarbonyl, (C₁-C₂₀)-alkoxycarbonyl, (C₆-C₁₂)-aryloxycarbonyl, (C₇-C₁₆)-aralkoxycarbonyl, (C₃-C₈)-cycloalkoxycarbonyl, (C₂-C₂₀)-alkenyloxycarbonyl, retinyloxycarbonyl, (C₂-C₂₀)-alkynyloxycarbonyl, (C₁-C₁₂)-alkylcarbonyloxy, (C₃-C₈)-cycloalkylcarbonyloxy, (C₆-C₁₂)-arylcarbonyloxy, (C₇-C₁₆)-aralkylcarbonyloxy, cinnamoyloxy, (C₂-C₁₂)-alkenylcarbonyloxy, (C₂-C₁₂)-alkynylcarbonyloxy, (C₁-C₁₂)-alkoxycarbonyloxy, (C₆-C₁₂)-aryloxycarbonyloxy, (C₇-C₁₆)-aralkyloxycarbonyloxy, (C₃-C₈)-cycloalkoxycarbonyloxy, (C₂-C₁₂)-alkenyloxycarbonyloxy, (C₂-C₁₂)-alkynyloxycarbonyloxy, carbamoyl, N—(C₁-C₁₂)-alkylcarbamoyl, N,N-di-(C₁-C₁₂)-alkylcarbamoyl, N—(C₃-C₈)-cycloalkylcarbamoyl, N,N-dicyclo-(C₃-C₈)-alkylcarbamoyl, N—(C₁-C₁₀)-alkyl-N—(C₃-C₈)-cycloalkylcarbamoyl, N—((C₃-C₈)-cycloalkyl-(C₁-C₆)-alkyl)-carbamoyl, N-(+)-dehydroabietylcarbamoyl, N—(C₁-C₆)-alkyl-N-(+)-dehydroabietylcarbamoyl, N—(C₆-C₁₂)-arylcarbamoyl, N—(C₇-C₁₆)-aralkylcarbamoyl, N—(C₁-C₁₀)-alkyl-N—(C₆-C₁₆)-arylcarbamoyl, N—(C₁-C₁₀)-alkyl-N—(C₇-C₁₆)-aralkylcarbamoyl, carbamoyloxy, N—(C₁-C₁₂)-alkylcarbamoyloxy, N,N-di-(C₁-C₁₂)-alkylcarbamoyloxy, N—(C₃-C₈)-cycloalkylcarbamoyloxy, N—(C₆-C₁₂)-arylcarbamoyloxy, N—(C₇-C₁₆)-aralkylcarbamoyloxy, N—(C₁-C₁₀)-alkyl-N—(C₆-C₁₂)-arylcarbamoyloxy, N—(C₁-C₁₀)-alkyl-N—(C₇-C₁₆)-aralkylcarbamoyloxy, N—((C₁-C₁₀)-alkyl)-carbamoyloxy, N—(C₁-C₁₀)-alkyl-N—((C₇-C₁₆)-aralkyloxy-(C₁-C₁₀)-alkyl)-carbamoyloxyamino, (C₁-C₁₂)-alkylamino, di-(C₁-C₁₂)-alkylamino, (C₃-C₈)-cycloalkylamino, (C₃-C₁₂)-alkenylamino, (C₃-C₁₂)-alkynylamino, N—(C₆-C₁₂)-arylamino, N—(C₇-C₁₁)-aralkylamino, N-alkyl-aralkylamino, N-alkyl-arylamino, (C₁-C₁₂)-alkoxyamino, (C₁-C₁₂)-alkoxy-N—(C₁-C₁₀)-alkylamino, (C₁-C₁₂)-alkanoylamino, (C₃-C₈)-cycloalkanoylamino, (C₆-C₁₂)-aroylamino, (C₇-C₁₆)-aralkanoylamino, (C₁-C₁₂)-alkanoyl-N—(C₁-C₁₀)-alkylamino, (C₃-C₈)-cycloalkanoyl-N—(C₁-C₁₀)-alkylamino, (C₆-C₁₂)-aroyl-N—(C₁-C₁₀)-alkylamino, (C₇-C₁₁)-aralkanoyl-N—(C₁-C₁₀)-alkylamino, amino-(C₁-C₁₀)-alkyl, (C₁-C₂₀)-alkylmercapto, (C₁-C₂₀)-alkylsulfinyl, (C₁-C₂₀)-alkylsulfonyl, (C₆-C₁₂)-arylmercapto, (C₆-C₁₂)-arylsulfinyl, (C₆-C₁₂)-arylsulfonyl, (C₇-C₁₆)-aralkylmercapto, (C₇-C₁₆)-aralkylsulfinyl, (C₇-C₁₆)-aralkylsulfonyl, sulfamoyl, N—(C₁-C₁₀)-alkylsulfamoyl, N,N-di-(C₁-C₁₀)-alkylsulfamoyl, (C₃-C₈)-cycloalkylsulfamoyl, N—(C₆-C₁₂)-arylsulfamoyl, N—(C₇-C₁₆)-aralkylsulfamoyl, N—(C₁-C₁₀)-alkyl-N—(C₆-C₁₂)-arylsulfamoyl, N—(C₁-C₁₀)-alkyl-N—(C₇-C₁₆)-aralkylsulfamoyl, (C₁-C₁₀)-alkylsulfonamido, (C₇-C₁₆)-aralkylsulfonamido, and N—((C₁-C₁₀)-alkyl-(C₇-C₁₆)-aralkylsulfonamido; where an aryl radical may be substituted by 1 to 5 substituents selected from hydroxyl, halogen, cyano, trifluoromethyl, nitro, carboxyl, (C₂-C₁₆)-alkyl, (C₃-C₈)-cycloalkyl, (C₃-C₈)-cycloalkoxy, (C₆-C₁₂)-aryl, (C₇-C₁₆)-aralkyl, (C₂-C₁₆)-alkenyl, (C₂-C₁₂)-alkynyl, (C₁-C₁₆)-alkoxy, (C₁-C₁₆)-alkenyloxy, (C₆-C₁₂)-aryloxy, (C₇-C₁₆)-aralkyloxy, (C₁-C₈)-hydroxyalkyl, —O—[CH₂]ₓ—C_{f}H_{(2f+1-g)}F_{g}, —OCF₂Cl, and —OCF₂—CHFCl;
x is 0 to 3;
f is 1 to 8; and
g is 0 or 1 to (2f+1);
including the physiologically active salts, esters, and prodrugs derived therefrom.

In certain embodiments, the compound is a compound of Formula II wherein
B is CO₂H;
R³⁰ and R³¹ are hydrogen;
R³² is selected from hydrogen, halo, aryl, substituted aryl, aryloxy, and substituted aryloxy;
R³³ is selected from hydrogen, halo, cyano, alkyl, alkynyl, and heteroaryl;
one of D or M is —S—, and the other is ═C(R³⁴)—; and
R³⁴ is hydrogen, aryl, or substituted aryl;
including the physiologically active salts, esters, and prodrugs derived therefrom.

As discussed herein, cyclic carboxamides are particularly suited for use in the present invention. However, use of other compounds that inhibit HIF prolyl hydroxylase activity is specifically contemplated. Such compounds are have been identified and are well-known in the art. For example, compounds according to the invention can include phenanthrolines and iron chelators, etc. In one embodiment, the compound for use in the present invention is an iron chelator, e.g., a hydroxamate. In particular embodiments, hydroxamates for use in the methods of the invention are selected from a compound of the formula (III)

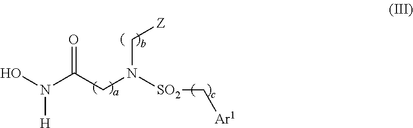

or pharmaceutically acceptable salts thereof, wherein:
a is an integer from 1 to 4;
b is an integer from 0 to 4;
c is an integer from 0 to 4;
Z is selected from the group consisting of (C₃-C₁₀) cycloalkyl, (C₃-C₁₀) cycloalkyl independently substituted with one or more Y¹, 3-10 membered heterocycloalkyl and 3-10 membered heterocycloalkyl independently substituted with one or more Y¹; (C₅-C₂₀) aryl, (C₅-C₂₀) aryl independently substituted with one or more Y¹, 5-20 membered heteroaryl and 5-20 membered heteroaryl independently substituted with one or more Y¹;
Ar¹ is selected from the group consisting of (C₅-C₂₀) aryl, (C₅-C₂₀) aryl independently substituted with one or more Y², 5-20 membered heteroaryl and 5-20 membered heteroaryl independently substituted with one or more Y²;
each Y¹ is independently selected from the group consisting of a lipophilic functional group, (C₅-C₂₀) aryl, (C₆-C₂₆) alkaryl, 5-20 membered heteroaryl and 6-26 membered alk-heteroaryl;
each Y² is independently selected from the group consisting of —R′, —OR′, —OR″, —SR′, —SR″, —NR′R′, —NO₂, —CN, -halogen, -trihalomethyl, trihalomethoxy, —C(O)R′, —C(O)OR′, —C(O)NR′R′, —C(O)NR′OR′, —C(NR′R′)═NOR′, —NR′—C(O)R′, —SO₂R′, —SO₂R″, —NR′—SO₂—R′, —NR′—C(O)—NR′R′, tetrazol-5-yl, —NR′—C(O)—OR′, —C(NR′R′)═NR′, —S(O)ₙ—R′, —S(O)ₙ—R″, and —NR′—C(S)—NR′R′; and
each R′ is independently selected from the group consisting of —H, (C₁-C₈) alkyl, (C₂-C₈) alkenyl, and (C₂-C₈) alkynyl; and
each R″ is independently selected from the group consisting of (C₅-C₂₀) aryl and (C₅-C₂₀) aryl independently substituted with one or more —OR′, —SR′, —NR′R′, —NO₂, —CN, halogen or trihalomethyl groups,
or wherein c is 0 and Ar′ is an N′ substituted urea-aryl, the compound has the structural formula (IIIa):

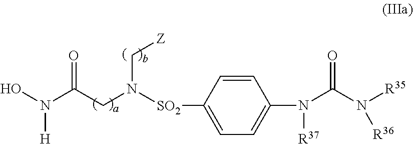

or pharmaceutically acceptable salts thereof, wherein:
   a, b, and Z are as defined above; and
   R³⁵ and R³⁶ are each independently selected from the group consisting of hydrogen, (C₁-C₈) alkyl, (C₂-C₈) alkenyl, (C₂-C₈) alkynyl, (C₃-C₁₀) cycloalkyl, (C₅-C₂₀) aryl, (C₅-C₂₀) substituted aryl, (C₆-C₂₆) alkaryl, (C₆-C₂₆) substituted alkaryl, 5-20 membered heteroaryl, 5-20 membered substituted heteroaryl, 6-26 membered alk-heteroaryl, and 6-26 membered substituted alk-heteroaryl; and
   R³⁷ is independently selected from the group consisting of hydrogen, (C₁-C₈) alkyl, (C₂-C₈) alkenyl, and (C₂-C₈) alkynyl.

As discussed previously, compounds according to the present invention include isoquinoline carboxamides. In certain embodiments, the compounds used in the present invention are as disclosed in International Publication No. WO 2004/108681, represented by formula (IV):

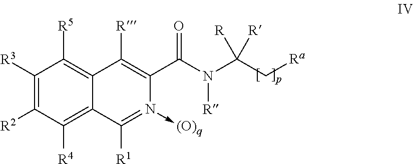

wherein:
q is zero or one;
p is zero or one;
R^{a} is —COOH or —WR⁸; provided that when R^{a} is —COOH then p is zero and when W is —WR⁸ then p is one;
W is selected from the group consisting of oxygen, —S(O)ₙ— and —NR⁹— where n is zero, one or two, R⁹ is selected from the group consisting of hydrogen, alkyl, substituted alkyl, acyl, aryl, substituted aryl, heteroaryl, substituted heteroaryl, heterocyclic and substituted heterocyclic and R⁸ is selected from the group consisting of hydrogen, alkyl, substituted alkyl, aryl, substituted aryl, heteroaryl, substituted heteroaryl, heterocyclic and substituted heterocyclic, or when W is —NR⁹— then R⁸ and R⁹, together with the nitrogen atom to which they are bound, can be joined to form a heterocyclic or a substituted heterocyclic group, provided that when W is —S(O)ₙ— and n is one or two, then R⁸ is not hydrogen;
R¹ is selected from the group consisting of hydrogen, alkyl, substituted alkyl, alkoxy, substituted alkoxy, amino, substituted amino, aminoacyl, aryl, substituted aryl, halo, heteroaryl, substituted heteroaryl, heterocyclic, substituted heterocyclic, and —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl or, when X is —NR⁷—, then R⁷ and R⁸, together with the nitrogen atom to which they are bound, can be joined to form a heterocyclic or substituted heterocyclic group;
R² and R³ are independently selected from the group consisting of hydrogen, alkyl, substituted alkyl, aryl, substituted aryl, heteroaryl, substituted heteroaryl, halo, hydroxy, cyano, —S(O)ₙN(R⁶)—R⁶ where n is 0, 1, or 2, —NR⁶C(O)NR⁶R⁶, —XR⁶ where X is oxygen, —S(O)ₙ— or —NR⁷— where n is zero, one or two, each R⁶ is independently selected from the group consisting of hydrogen, alkyl, substituted alkyl, aryl, substituted aryl, cycloalkyl, substituted cycloalkyl, heteroaryl, substituted heteroaryl, heterocyclic and substituted heterocyclic provided that when X is —SO— or —SO₂—, then R⁶ is not hydrogen, and R⁷ is selected from the group consisting of hydrogen, alkyl, aryl, or R², R³ together with the carbon atom pendent thereto, form an aryl substituted aryl, heteroaryl, or substituted heteroaryl;
R⁴ and R⁵ are independently selected from the group consisting of hydrogen, halo, alkyl, substituted alkyl, alkoxy, substituted alkoxy, aryl, substituted aryl, heteroaryl, substituted heteroaryl and —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl or, when X is —NR⁷—, then R⁷ and R⁸, together with the nitrogen atom to which they are bound, can be joined to form a heterocyclic or substituted heterocyclic group;
R is selected from the group consisting of hydrogen, deuterium and methyl;
R′ is selected from the group consisting of hydrogen, deuterium, alkyl and substituted alkyl; alternatively, R and R′ and the carbon pendent thereto can be joined to form cycloalkyl, substituted cycloalkyl, heterocyclic or substituted heterocyclic group;
R″ is selected from the group consisting of hydrogen and alkyl or R″ together with R′ and the nitrogen pendent thereto can be joined to form a heterocyclic or substituted heterocyclic group;

R′″ is selected from the group consisting of hydroxy, alkoxy, substituted alkoxy, acyloxy, cycloalkoxy, substituted cycloalkoxy, aryloxy, substituted aryloxy, heteroaryloxy, substituted heteroaryloxy, aryl, —S(O)ₙ—R¹⁰ wherein R¹⁰ is selected from the group consisting of alkyl, substituted alkyl, cycloalkyl, substituted cycloalkyl, aryl, substituted aryl, heteroaryl and substituted heteroaryl and n is zero, one or two;
and pharmaceutically acceptable salts, esters and prodrugs thereof.

In an alternative embodiment, the compounds of formula (IV) are represented by formula (IVA):

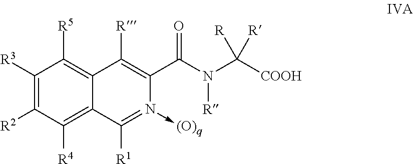

wherein R¹, R², R³, R⁴, R⁵, R, R′, R″, R′″ and q are as defined above; and
pharmaceutically acceptable salts, esters, prodrugs thereof.

In another alternative embodiment, the compounds of formula (IV) are represented by the formula (IVB):

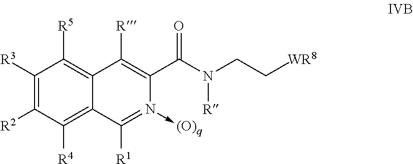

wherein R¹, R², R³, R⁴, R⁵, R″, R′″, WR⁸ and q are as defined above; and
pharmaceutically acceptable salts, esters, prodrugs thereof.

In another alternative embodiment, the invention is directed to compounds represented by the formula (IVC):

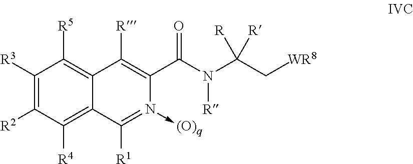

wherein R¹, R², R³, R⁴, R⁵, R, R′, R″, R′″, WR⁸ and q are as defined above; and
pharmaceutically acceptable salts, esters, prodrugs thereof.

In yet another alternative embodiment, the invention is directed to compounds represented by the formula (IVD):

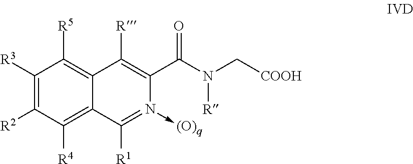

wherein R¹, R², R³, R⁴, R⁵, R, R′, R″, R′″ and q are as defined above; and
pharmaceutically acceptable salts, esters, prodrugs thereof.

In other embodiments, the invention is directed to isoquinoline carboxamide compounds represented by the formulae (VA), (VB), (VC), (V), wherein said formulae are defined below.

Formula VA:

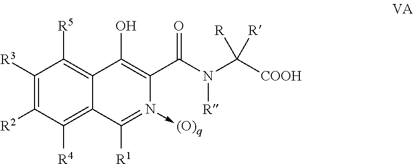

wherein:
q is zero or one;
R¹ is selected from the group consisting of hydrogen, alkyl, substituted alkyl, alkoxy, substituted alkoxy, aryl, substituted aryl, halo, heteroaryl, substituted heteroaryl, heterocyclic, substituted heterocyclic, and —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl;
R² and R³ are independently selected from the group consisting of hydrogen, alkyl, substituted alkyl, alkoxy, substituted alkoxy, aryl, substituted aryl, heteroaryl, substituted heteroaryl, halo, hydroxy, cyano, —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl;
R⁴ and R⁵ are independently selected from the group consisting of hydrogen, halo, alkyl, substituted alkyl, alkoxy, substituted alkoxy, aryl, substituted aryl, heteroaryl, substituted heteroaryl and —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl;
R is selected from the group consisting of hydrogen and methyl;
R′ is selected from the group consisting of alkyl and substituted alkyl; or R and R′ may be joined to form a cycloalkyl, substituted cycloalkyl, heterocyclic or substituted heterocyclic;
R″ is selected from the group consisting of hydrogen and alkyl or R″ together with R′ and the nitrogen pendent thereto forms a heterocyclic or substituted heterocyclic group;
or pharmaceutically acceptable salts and/or prodrugs thereof.

Formula VB:

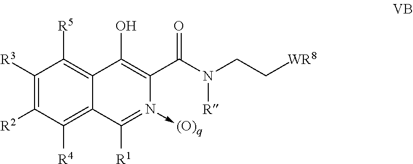

wherein:
q is zero or one;
W is selected from the group consisting of oxygen, —S(O)ₙ— and —NR⁹— where n is zero, one or two, R⁹ is selected from the group consisting of hydrogen, alkyl, substituted alkyl, acyl, aryl, substituted aryl, heteroaryl, substituted heteroaryl, heterocyclic and substituted heterocyclic, and R⁸ is selected from the group consisting of hydrogen, alkyl, substituted alkyl, aryl, substituted aryl, heteroaryl, substituted heteroaryl, heterocyclic and substituted heterocyclic;
R″ is selected from hydrogen and alkyl;
R¹ is selected from the group consisting of hydrogen, alkyl, substituted alkyl, alkoxy, substituted alkoxy, aryl, substituted aryl, halo, heteroaryl, substituted heteroaryl, heterocyclic, substituted heterocyclic, and —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl;
R² and R³ are independently selected from the group consisting of hydrogen, alkyl, substituted alkyl, alkoxy, substituted alkoxy, aryl, substituted aryl, heteroaryl, substituted heteroaryl, halo, hydroxy, cyano, —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl;
R⁴ and R⁵ are independently selected from the group consisting of hydrogen, halo, alkyl, substituted alkyl, alkoxy, substituted alkoxy, aryl, substituted aryl, heteroaryl, substituted heteroaryl and —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl; or
pharmaceutically acceptable salts and/or prodrugs thereof.

Formula VC:

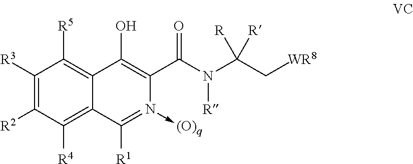

wherein:
q is zero or one;
R¹ is selected from the group consisting of hydrogen, alkyl, substituted alkyl, alkoxy, substituted alkoxy, aryl, substituted aryl, halo, heteroaryl, substituted heteroaryl, heterocyclic, substituted heterocyclic, and —XR⁶ where X is oxygen, —S(O)ₙ or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl, or aryl;
R² and R³ are independently selected from the group consisting of hydrogen, alkyl, substituted alkyl, alkoxy, substituted alkoxy, aryl, substituted aryl, heteroaryl, substituted heteroaryl, halo, hydroxy, cyano, —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl, or aryl;
R⁴ and R⁵ are independently selected from the group consisting of hydrogen, halo, alkyl, substituted alkyl, alkoxy, substituted alkoxy, aryl, substituted aryl, heteroaryl, substituted heteroaryl and —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl, or aryl;
R is selected from the group consisting of hydrogen and methyl;
R′ is selected from the group consisting of alkyl and substituted alkyl; or R and R′ can be joined to form cycloalkyl, substituted cycloalkyl, heterocyclic or substituted heterocyclic,
R″ is selected from the group consisting of hydrogen and alkyl or R″ together with R′ and the nitrogen pendent thereto forms a heterocyclic or substituted heterocyclic group;
W is selected from the group consisting of oxygen, —S(O)ₙ— and —NR⁹— where n is zero, one or two, R⁹ is selected from the group consisting of hydrogen, alkyl, substituted alkyl, aryl, substituted aryl, heteroaryl, substituted heteroaryl, heterocyclic and substituted heterocyclic, and R⁸ is selected from the group consisting of hydrogen, alkyl, substituted alkyl, aryl, substituted aryl, heteroaryl, substituted heteroaryl, heterocyclic and substituted heterocyclic; or
pharmaceutically acceptable salts and/or prodrugs thereof.

Formula VD:

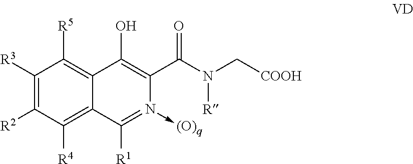

wherein:
q is zero or one;
R″ is selected from hydrogen and alkyl;
R¹ is selected from the group consisting of hydrogen, alkyl, substituted alkyl, alkoxy, substituted alkoxy, aryl, substituted aryl, halo, heteroaryl, substituted heteroaryl, heterocyclic, substituted heterocyclic, and —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl;
R² and R³ are independently selected from the group consisting of hydrogen, alkyl, substituted alkyl, aryl, substituted aryl, heteroaryl, substituted heteroaryl, halo, hydroxy, cyano, —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl;
R⁴ and R⁵ are independently selected from the group consisting of hydrogen, halo, alkyl, substituted alkyl, alkoxy, substituted alkoxy, aryl, substituted aryl, heteroaryl, substituted heteroaryl and —XR⁶ where X is oxygen, —S(O)ₙ— or —NR⁷— where n is zero, one or two, R⁶ is selected from the group consisting of alkyl, substituted alkyl, aryl, substituted aryl, heteroaryl, substituted heteroaryl, heterocyclic and substituted heterocyclic, and R⁷ is hydrogen, alkyl or aryl; or
pharmaceutically acceptable salts and/or prodrugs thereof.

In compounds of formulae (IV), (IVA), (IVB), (IVC), and (IVD), preferably R¹ is selected from the group consisting of hydrogen, alkyl, substituted alkyl, halo, alkoxy, aryloxy, substituted aryloxy, substituted aryl, alkylthio, aminoacyl, aryl, substituted amino, heteroaryl, heteroaryloxy, —S(O)ₙ-aryl, —S(O)ₙ-substituted aryl, —S(O)ₙ-heteroaryl, and —S(O)ₙ-substituted heteroaryl, where n is zero, one or two.

More preferably, R¹ is selected from the group consisting of: (3-methoxyphenyl)sulfanyl; (4-chlorophenyl)sulfanyl; (4-methylphenyl)sulfanyl; 2-fluorophenoxy; 2-methoxyphenoxy; (2-methoxyphenyl)sulfanyl 3-fluorophenoxy; 3-methoxyphenoxy; 4-(methylcarbonylamino)phenoxy; 4-(methylsulfonamido)phenoxy; 4-fluorophenoxy; 4-methoxyphenoxy; 4-methoxyphenylsulfanyl; 4-methylphenyl; bromo; chloro; dimethylaminomethyl; ethoxy; ethylsulfanyl; hydrogen; isopropyl; methoxy; methoxymethyl; methyl; N,N-dimethylaminocarbonyl; naphth-2-yloxy; naphthylsulfanyl; phenoxy; phenyl; phenylamino; phenylsulfinyl; phenylsulfanyl; pyridin-2-yloxy; pyridin-2-yl; and pyridin-2-ylsulfanyl.

In compounds of formulae (IV), (IVA), (IVB), (IVC), and (IVD), R² is preferably selected from the group consisting of substituted amino, aryloxy, substituted aryloxy, alkoxy, substituted alkoxy, halo, hydrogen, alkyl, substituted alkyl, aryl, —S(O)ₙ-aryl, —S(O)ₙ-substituted aryl, —S(O)ₙ-cycloalkyl, where n is zero, one or two, aminocarbonylamino, heteroaryloxy, and cycloalkyloxy. More preferably, R² is selected from the group consisting of: (4-methoxy)phenylsulfonylamino; 2,6-dimethylphenoxy; 3,4-difluorophenoxy; 3,5-difluorophenoxy; 3-chloro-4-fluorophenoxy; 3-methoxy-4-fluorophenoxy; 3-methoxy-5-fluorophenoxy; 4-(methylsulfonamido)phenoxy; 4-(phenylsulfonamido)phenoxy; 4-CF₃—O-phenoxy; 4-CF₃-phenoxy; 4-chlorophenoxy; 4-fluorophenoxy; 4-(4-fluorophenoxy)phenoxy; 4-methoxyphenoxy; 4-nitrophenoxy; benzyloxy; bromo; butoxy; CF₃; chloro; cyclohexyloxy; cyclohexylsulfanyl; cyclohexylsulfonyl; fluoro; hydrogen; iodo; isopropoxy; methyl; phenoxy; phenyl; phenylsulfanyl; phenylsulfinyl; phenylsulfonyl; phenylurea; pyridin-1-ylsulfanyl; pyridin-3-yloxy; and pyridin-4-ylsulfanyl.

In compounds of formulae (IV), (IVA), (IVB), (IVC), and (IVD), R³ is preferably selected from the group consisting of: substituted aryloxy, substituted alkoxy, alkoxy, substituted alkyl, alkyl, amino, cycloalkyloxy, hydrogen, halo, aryl, —S(O)ₙ-aryl, —S(O)ₙ-substituted aryl, —S(O)ₙ-heteroaryl, and —S(O)ₙ-substituted heteroaryl, where n is zero, one or two, aminocarbonylamino, and heteroaryloxy.

More preferably, R³ is selected from the group consisting of: amino; (4-methyl)phenylsulfonylaminophenoxy; 3,4-difluorophenoxy; 3,5-difluorophenoxy; 3-fluoro-5-methoxy-phenoxy; 3-chloro-4-fluorophenoxy; 4-CF₃—O-phenoxy; 4-CF₃-phenoxy; 4-chlorophenoxy; 4-fluorophenoxy; 4-(4-fluorophenoxy)phenoxy; 4-methoxyphenoxy; benzyloxy; bromo; butoxy; CF₃; chloro; cyclohexyloxy; hydrogen; iodo; isopropoxy; phenoxy; phenyl; phenylsulfanyl; phenylsulfonyl; phenylsulfinyl; phenylurea; pyridin-1-ylsulfanyl; pyridin-3-yloxy; and pyridin-4-ylsulfanyl.

Alternatively, Wand R³, combined with the carbon atoms pendent thereto, are joined to form an aryl group. Preferably, the aryl group is phenyl.

In compounds of formulae (IV), (IVA), (IVB), (IVC), and (IVD), R⁴ is preferably selected from the group consisting of: substituted arylthio, halo, hydrogen, substituted alkyl and aryl.

More preferably, R⁴ is selected from the group consisting of: 4-chlorophenyl sulfanyl; chloro; hydrogen; methoxymethyl; and phenyl.

In compounds of formulae (IV), (IVA), (IVB), (IVC), and (IVD), R⁵ is preferably hydrogen or aryl. More preferably R⁵ is hydrogen or phenyl.

In compounds of formulae (IV), (IVA) and (IVC), R is preferably selected from the group consisting of hydrogen, deuterium, aryl and alkyl. More preferably R is selected from the group consisting of phenyl, hydrogen, deuterium and methyl.

In compounds of formulae (IV), (IVA) and (IVC), R′ is selected from the group consisting of preferably hydrogen, deuterium, alkyl, substituted alkyl, and substituted amino. More preferably, R′ is selected from the group consisting of: 4-aminobutyl; 4-hydroxybenzyl; benzyl; carboxylmethyl; deuterium; hydroxymethyl; imidazol-4-ylmethyl; isopropyl; methyl; and propyl.

Alternatively, R, R′ and the carbon atom pendent thereto join to form a cycloalkyl and more preferably cyclopropyl.

In compounds of formulae (IV), (IVA) and (IVC), R″ is preferably hydrogen, alkyl or substituted alkyl. More preferably, R″ is hydrogen, methyl or carboxylmethyl (—CH₂C(O)OH). Alternatively, R′, R″ and the carbon atom and nitrogen atom respectively pendent thereto join to form a heterocyclic group and more preferably pyrrolidinyl.

In compounds of formulae (IV), (IVA), (IVB), (IVC) and (IVD), preferably R′″ is selected from the group consisting of hydrogen, hydroxy, alkoxy, substituted alkoxy, cycloalkoxy, substituted cycloalkoxy, thiol, acyloxy and aryl. Preferably, R′″ is selected from the group consisting of: hydroxy; benzyloxy; ethoxy; thiol; methoxy; methylcarbonyloxy; and phenyl.

In compounds of formulae (IV), (IVB) and (IVC), WR⁸ is preferably selected from the group consisting of amino, substituted amino, aminoacyl, hydroxy, and alkoxy. More preferably, WR⁸ is selected from the group consisting of: amino; dimethylamino; hydroxy; methoxy; and methylcarbonylamino.

In certain embodiments, the compounds for use in the invention are thiochromene-3-carboxamides. In one embodiment, a compound for use in the methods of the present invention is {[4-Hydroxy-7-(4-methoxy-phenyl)-2-oxo-2H-thiochromene-3-carbonyl]-amino}-acetic acid (Compound L) or [(7-Butoxy-4-hydroxy-2-oxo-2H-thiochromene-3-carbonyl)-amino]-acetic acid (Compound M). In particular embodiments, the thiochromene-3-carboxamide is a compound of formula VII:

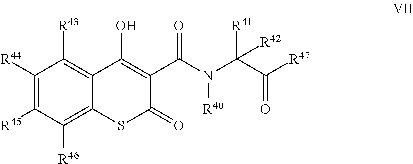

R⁴⁰ is selected from the group consisting of hydrogen, alkyl, and substituted alkyl;
R⁴¹ is selected from the group consisting of hydrogen, deuterium, alkyl, and substituted alkyl;
R⁴² is selected from the group consisting of hydrogen, deuterium, and methyl;
R⁴³, R⁴⁴, R⁴⁵ and R⁴⁶ are independently selected from the group consisting of hydrogen, hydroxy, cyano, halo, nitro, acyl, amino, substituted amino, acylamino, sulfonyl, substituted sulfonyl, alkyl, substituted alkyl, alkenyl, substituted alkenyl, alkynyl, substituted alkynyl, alkoxy, substituted alkoxy, cycloalkyloxy, substituted cycloalkyloxy, heterocyclyloxy, substituted heterocyclyloxy, aryl, substituted aryl, aryloxy, substituted aryloxy, heteroaryloxy, substituted heteroaryloxy, alkylthio, substituted alkylthio, cycloalkylthio, substituted cycloalkylthio, arylthio, substituted arylthio, heteroarylthio, substituted heteroarylthio, hetereocyclicthio, substituted heterocyclicthio, heteroaryl, and substituted heteroaryl;
or R⁴³ and R⁴⁴, R⁴⁴ and R⁴⁵, or R⁴⁵ and R⁴⁶ are taken together with the carbon atoms to which they are attached to form a 5- or 6-membered aryl or substituted aryl;
R⁴⁷ is —NR⁴⁸R⁴⁹ or —OR⁵⁰;
R⁴⁸ and R⁴⁹ are independently selected from the group consisting hydrogen, alkyl, alkylene-cycloalkyl, heterocyclic, and aryl;
or R⁴⁸ and R⁴⁹ are taken together with the nitrogen to which they are attached form a 5- or 6-membered heterocyclic or substituted heterocyclic; and
R⁵⁰ is selected from the group consisting of hydrogen, and alkyl which is unsubstituted or substituted with one or more substituents independently selected from the group consisting of cycloalkyl, heterocyclic, aryl, and heteroaryl;
or a pharmaceutically acceptable salt and/or prodrug thereof.

In some embodiments, the thiochromene-3-carboxamide is a compound of the formula VII wherein:
R⁴⁰ and R⁴² are hydrogen;
R⁴¹ is selected from the group consisting of hydrogen or methyl;
R⁴³, R⁴⁴ R⁴⁵ and R⁴⁶ are independently selected from the group consisting of hydrogen, halo, (C₁-C₄)-alkyl, aryl, (C₁-C₄)-alkoxy, and heteroaryl; wherein alkyl, aryl, and heteroaryl substituents are optionally substituted with 1 or 2 substituents independently selected from the group consisting of halo, (C₁-C₄)-alkyl, and (C₁-C₄)-alkoxy; and
R⁴⁷ is —OR⁵⁰ and R⁵⁰ is hydrogen.

As discussed, supra, compounds according the present invention are in some embodiments heterocyclic carboxamides; in particular, pyrrolopyridazine carboxamides. In one embodiment, a compound for use in the methods of the present invention is {[4-Hydroxy-2-oxo-1-(4-trifluoromethyl-benzyl)-1,2-dihydro-pyrrolo[1,2-b]pyridazine-3-carbonyl]-amino}-acetic acid (Compound G), (S)-2-{[6-Chloro-4-hydroxy-2-oxo-1-(4-trifluoromethyl-benzyl)-1,2-dihydro-pyrrolo[1,2-b]pyridazine-3-carbonyl]-amino}-propionic acid (Compound H), or {[6-Chloro-1-(4-chloro-benzyl)-4-hydroxy-2-oxo-1,2-dihydro-pyrrolo[1,2-b]pyridazine-3-carbonyl]-amino}-acetic acid (Compound I). In certain embodiments, compounds for use in the invention are pyrrolopyridazine-3-carboxamides. In one embodiment, the compound is a compound of formula VIII:

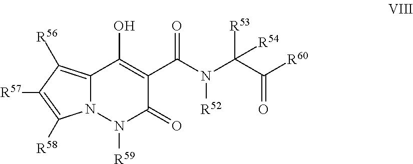

wherein
R⁵² is selected from the group consisting of hydrogen, alkyl, and substituted alkyl;
R⁵³ is selected from the group consisting of hydrogen, deuterium, alkyl, and substituted alkyl;
R⁵⁴ is selected from the group consisting of hydrogen, deuterium, alkyl, and substituted alkyl;
R⁵⁶, R⁵⁷ and R⁵⁸ independently are selected from the group consisting of hydrogen, hydroxy, cyano, halo, nitro, acyl, amino, substituted amino, acylamino, sulfonyl, substituted sulfonyl, alkyl, substituted alkyl, alkenyl, substituted alkenyl, alkynyl, substituted alkynyl, alkoxy, substituted alkoxy, cycloalkyl, substituted cycloalkyl, cycloalkyloxy, substituted cycloalkyloxy, heterocyclyloxy, substituted heterocyclyloxy, carboxyl, carboxyl ester, carboxylamide, oxycarbonylamino, aminocarbonyloxy, aminocarbonylamino, aryl, substituted aryl, aryloxy, substituted aryloxy, heteroaryloxy, substituted heteroaryloxy, alkylthio, substituted alkylthio, cycloalkylthio, substituted cycloalkylthio, arylthio, substituted arylthio, heteroarylthio, substituted heteroarylthio, hetereocyclicthio, substituted heterocyclicthio, heteroaryl, and substituted heteroaryl;
or wherein R⁵⁶ and R⁵⁷, or R⁵⁷ and R⁵⁸, together with the carbons to which they are attached, form a carbocyclic 5- or 6-membered aromatic ring, optionally substituted by one or two hydrogen, halogen, alkyl, substituted alkyl, alkoxy, substituted alkoxy, aryl, or substituted aryl;
R⁵⁹ is selected from the group consisting of hydrogen, alkyl, substituted alkyl, alkenyl, substituted alkenyl, alkynyl, substituted alkynyl, cycloalkyl, substituted cycloalkyl, heterocyclic, substituted heterocyclic, aryl, substituted aryl, heteroaryl, and substituted heteroaryl;
R⁶⁰ is —NR⁶¹R⁶² or —OR⁶³;
R⁶¹ and R⁶² independently are selected from the group consisting hydrogen, alkyl, alkylene-cycloalkyl, C₃-C₈ heterocyclic, aryl, and —C(O)(C₁-C₄ alkyl);
or R⁶¹ and R⁶² taken together with the nitrogen to which they are attached form a 5- or 6-membered heterocyclic or substituted heterocyclic; and
R⁶³ is selected from the group consisting of hydrogen and alkyl which is unsubstituted or substituted with one or more substituents independently selected from the group consisting of cycloalkyl, heterocyclic, aryl, and heteroaryl;
or a pharmaceutically acceptable salt, single stereoisomer, mixture of stereoisomers, ester, tautomer or prodrug thereof.

In certain embodiments, the pyrrolopyridazine-3-carboxamide is a compound of formula VIII wherein
R⁵² and R⁵³ are hydrogen;
R⁵⁴ is selected from the group consisting of hydrogen and methyl;
R⁵⁶, R⁵⁷, and R⁵⁸ independently are selected from the group consisting of hydrogen, halo, and aryl;
R⁵⁹ is selected from the group consisting of hydrogen, alkyl, —CH₂-aryl, —CH₂-substituted aryl, or —C₁₋₁₂-heteroaryl; and
R⁶⁰ is —OR⁶³; wherein R⁶³ is hydrogen or alkyl.

In particular embodiments, the pyrrolopyridazine-3-carboxamide is a compound of formula VIII wherein
R⁵², R⁵³, and R⁵⁴ are hydrogen;
R⁵⁶ and R⁵⁸ independently are selected from the group consisting of hydrogen and halo;
R⁵⁷ is selected from the group consisting of hydrogen, halo, and aryl;
R⁵⁹ is selected from the group consisting of hydrogen, alkyl, —CH₂-aryl, —CH₂-substituted aryl, or —CH₂-heteroaryl; and
R⁶⁰ is —OR⁶³; wherein R⁶³ is hydrogen or alkyl.

As discussed, supra, compounds according the present invention are in some embodiments heterocyclic carboxamides; in particular, pyrrolopyridine carboxamides. In one embodiment, a compound for use in the methods of the present invention is {[7-Cyano-1-(2-fluoro-benzyl)-4-hydroxy-1H-pyrrolo[2,3-c]pyridine-5-carbonyl]-amino}-acetic acid (Compound O), [(1-Biphenyl-4-ylmethyl-7-cyano-4-hydroxy-1H-pyrrolo[2,3-c]pyridine-5-carbonyl)-amino]-acetic acid (Compound P), or {[2,3-Dichloro-7-cyano-4-hydroxy-1-(4-methoxy-benzyl)-1H-pyrrolo[2,3-c]pyridine-5-carbonyl]-amino}-acetic acid (Compound Q). In certain embodiments, compounds for use in the invention are pyrrolopyridine-5-carboxamides or pyrrolopyridine-6-carboxamides. In one embodiment, the compound is a compound of formula IX:

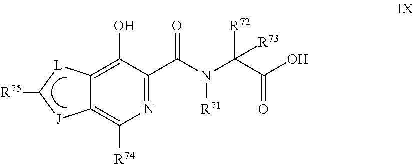

wherein
one of L or J is —N(R⁷⁶)—, and the other is ═C(R⁷⁷)—;
R⁷¹ is selected from the group consisting of hydrogen, alkyl, and substituted alkyl;
R⁷² is selected from the group consisting of hydrogen, deuterium, and methyl;
R⁷³ is selected from the group consisting of hydrogen, deuterium, alkyl, and substituted alkyl;
R⁷⁴ is selected from the group consisting of hydrogen, halo, cyano, hydroxyl, alkyl, substituted alkyl, cycloalkyl, substituted cycloalkyl, alkenyl, substituted alkenyl, alkynyl, substituted alkynyl, alkoxy, substituted alkoxy, cycloalkoxy, substituted cycloalkoxy, aryl, substituted aryl, aryloxy, substituted aryloxy, heteroaryl, substituted heteroaryl, heterocyclyl, substituted heterocyclyl, heterocyclyloxy, substituted heterocyclyloxy, heteroaryloxy, substituted heteroaryloxy, acyl, aminoacyl, nitro, amino, substituted amino, acylamino, sulfanyl, sulfonyl, thioether, arylthio, and substituted arylthio;
R⁷⁵ and R⁷⁷ are each independently selected from the group consisting of hydrogen, halo, cyano, hydroxyl, alkyl, substituted alkyl, cycloalkyl, substituted cycloalkyl, alkenyl, substituted alkenyl, alkynyl, substituted alkynyl, alkoxy, substituted alkoxy, cycloalkoxy, substituted cycloalkoxy, aryl, substituted aryl, aryloxy, substituted aryloxy, heteroaryl, substituted heteroaryl, heterocyclyl, substituted heterocyclyl, heterocyclyloxy, substituted heterocyclyloxy, heteroaryloxy, substituted heteroaryloxy, acyl, aminoacyl, nitro, amino, substituted amino, acylamino, sulfanyl, sulfonyl, thioether, arylthio, and substituted arylthio;
or where when L or J is ═C(R⁷⁷)—, then R⁷⁵ and R⁷⁷ together with the carbon atoms bound thereto join to form a cycloalkenyl, substituted cycloalkenyl, aryl, substituted aryl, heteroaryl, or substituted heteroaryl; and
R⁷⁶ is selected from the group consisting of hydrogen, hydroxyl, alkyl, substituted alkyl, alkenyl, substituted alkenyl, alkynyl, substituted alkynyl, aryl, substituted aryl, heteroaryl, and substituted heteroaryl;
or pharmaceutically acceptable salts, single stereoisomers, mixtures of stereoisomers, esters, or prodrugs thereof.

In certain embodiments, the pyrrolopyridine-3-carboxamide is a compound of formula IX wherein
L is ═C(R⁷⁷)—;
J is —N(R⁷⁶)—;
R⁷¹, R⁷², and R⁷³ are hydrogen;
R⁷⁴ is selected from the group consisting of hydrogen, halo, cyano, alkyl, and aryl;
R⁷⁵ is selected from the group consisting of hydrogen, halo, cyano, alkyl, aryl, substituted aryl, aryloxy, substituted amino, heteroaryl, and substituted heteroaryl;
R⁷⁶ is selected from the group consisting of alkyl, substituted alkyl, aryl, and substituted aryl;
R⁷⁷ is selected from the group consisting of hydrogen, halo, cyano, alkyl, aryl, and substituted aryl; and
   or pharmaceutically acceptable salts, single stereoisomers, mixtures of stereoisomers, esters, or prodrugs thereof.

In another embodiment, the pyrrolopyridine-3-carboxamide is a compound of formula IX wherein
L is —N(R⁷⁶)—;
J is ═C(R⁷⁷)—;
R⁷¹, R⁷², and R⁷³ are hydrogen;
R⁷⁴ is selected from hydrogen, cyano, and alkyl;
R⁷⁵ and R⁷⁷ are selected from hydrogen or halogen;
R⁷⁶ is selected from the group consisting of hydrogen, alkyl, substituted alkyl, and aryl;
or pharmaceutically acceptable salts, single stereoisomers, mixtures of stereoisomers, esters, or prodrugs thereof.

As discussed, supra, compounds according the present invention are in some embodiments heterocyclic carboxamides; in particular, chromene carboxamides. In one embodiment, a compound for use in the methods of the present invention is {[4-Hydroxy-2-oxo-7-(4-phenoxy-phenyl)-2H-chromene-3-carbonyl]-amino}-acetic acid (Compound J) or [(6-Hexyloxy-4-hydroxy-2-oxo-2H-chromene-3-carbonyl)-amino]-acetic acid (Compound K). In certain embodiments, compounds for use in the invention are chromene-3-carboxamides. In one embodiment, the compound is a compound of formula X:

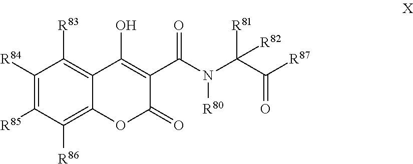

wherein
R⁸⁰ is selected from the group consisting of hydrogen, alkyl, and substituted alkyl;
R⁸¹ is selected from the group consisting of hydrogen, deuterium, alkyl, and substituted alkyl;
R⁸² is selected from the group consisting of hydrogen, deuterium, and methyl; and
R⁸³ and R⁸⁶ are independently selected from the group consisting of hydrogen, halo, alkyl, alkoxy, substituted alkoxy, cycloalkyloxy, substituted cycloalkyloxy, aryl, substituted aryl, heteroaryl, and substituted heteroaryl;
R⁸⁴ and R⁸⁵ are independently selected from the group consisting of hydrogen, hydroxy, cyano, halo, nitro, acyl, amino, substituted amino, acylamino, sulfonyl, substituted sulfonyl, alkyl, substituted alkyl, alkenyl, substituted alkenyl, alkynyl, substituted alkynyl, alkoxy, substituted alkoxy, cycloalkyloxy, substituted cycloalkyloxy, heterocyclyloxy, substituted heterocyclyloxy, aryl, substituted aryl, aryloxy, substituted aryloxy, heteroaryloxy, substituted heteroaryloxy, alkylthio, substituted alkylthio, cycloalkylthio, substituted cycloalkylthio, arylthio, substituted arylthio, heteroarylthio, substituted heteroarylthio, hetereocyclicthio, substituted heterocyclicthio, heteroaryl, and substituted heteroaryl;
or one of R⁸³ and R⁸⁴, R⁸⁴ and R⁸⁵, or R⁸⁵ and R⁸⁶ taken together with the carbon atoms to which they are attached optionally form an aryl, substituted aryl, cycloalkyl, substituted cycloalkyl, heterocyclic, substituted heterocyclic, heteroaryl or substituted heteroaryl;
R⁸⁷ is —NR⁸⁸R⁸⁹ or —OR⁹⁰;
R⁸⁸ and R⁸⁹ are independently selected from the group consisting hydrogen, alkyl, cycloalkyl-alkylene, (C₃-C₈)heterocyclic, aryl, —C(O)(C₁-C₄)alkyl, and alkyl-(C₃-C₈)cycloalkylene;
or R⁸⁸ and R⁸⁹ taken together with the nitrogen to which they are attached form a 5- or 6-membered heterocyclic or substituted heterocyclic; and
R⁹⁰ is selected from the group consisting of hydrogen, a cation, and alkyl which is unsubstituted or substituted with one or more substituents independently selected from the group consisting of cycloalkyl, heterocyclic, aryl, and heteroaryl;
or a pharmaceutically acceptable salt, single stereoisomer, mixture of stereoisomers, ester, or prodrug thereof.

In some embodiments, the chromene-3-carboxamide is a compound of Formula X wherein
R⁸⁰ and R⁸² are hydrogen;
R⁸¹ is selected from the group consisting of hydrogen, alkyl, and substituted alkyl;
R⁸³, R⁸⁴, R⁸⁵, and R⁸⁶ are independently selected from the group consisting of hydrogen, halo, alkyl, alkoxy, substituted alkoxy, cycloalkyloxy, substituted cycloalkyloxy, aryl, substituted aryl, heteroaryl, and substituted heteroaryl; and
R⁸⁷ is —OR⁹⁰; wherein R⁹⁰ is hydrogen, a cation or alkyl.

As discussed, supra, compounds according the present invention are in some embodiments heterocyclic carboxamides; in particular, naphthalene carboxamides. In one embodiment, a compound for use in the methods of the present invention is [(7-Chloro-1-hydroxy-4,4-dimethyl-3-oxo-3,4-dihydro-naphthalene-2-carbonyl)-amino]-acetic acid (Compound N). In certain embodiments, compounds for use in the invention are naphthalene-3-carboxamides. In one embodiment, the compound is a compound of formula XI:

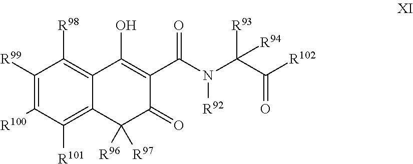

wherein
R⁹² is selected from the group consisting of hydrogen, alkyl, and substituted alkyl;
R⁹³ is selected from the group consisting of hydrogen, deuterium, alkyl, and substituted alkyl;
R⁹⁴ is selected from the group consisting of hydrogen, deuterium, and methyl;
R⁹⁶ and R⁹⁷ are independently selected from group consisting of hydrogen, alkyl, arylalkylene, and substituted arylalkylene;
or R⁹⁶ and R⁹⁷ together with the carbon atom attached thereto join to form a cycloalkyl or substituted cycloalkyl;
R⁹⁸ and R¹⁰¹ are independently selected from the group consisting of hydrogen, halo, and alkyl;
R⁹⁹ and R¹⁰⁰ are independently selected from the group consisting of hydrogen, hydroxy, cyano, halo, nitro, acyl, amino, substituted amino, acylamino, sulfonyl, substituted sulfonyl, alkyl, substituted alkyl, alkenyl, substituted alkenyl, alkynyl, substituted alkynyl, alkoxy, substituted alkoxy, aryl, substituted aryl, aryloxy, substituted aryloxy, alkylthio, substituted alkylthio, cycloalkylthio, substituted cycloalkylthio, arylthio, substituted arylthio, heteroarylthio, substituted heteroarylthio, hetereocyclicthio, substituted heterocyclicthio, heteroaryl, and substituted heteroaryl;
R¹⁰² is —NR¹⁰³R¹⁰⁴ or —OR¹⁰⁵;
R¹⁰³ and R¹⁰⁴ are independently selected from the group consisting hydrogen, alkyl, alkylene-cycloalkyl, C₃-C₈ heterocyclic, aryl, —C(O)(C₁-C₄ alkyl), and C₃-C₈ cycloalkylene-alkyl;
or R¹⁰³ and R¹⁰⁴ taken together with the nitrogen to which they are attached form a 5- or 6-membered heterocyclic or substituted heterocyclic; and
R¹⁰⁵ is selected from the group consisting of hydrogen, a cation, and alkyl which is unsubstituted or substituted with one or more substituents independently selected from the group consisting of cycloalkyl, heterocyclic, aryl, and heteroaryl;
   or a pharmaceutically acceptable salt, single stereoisomer, mixture of stereoisomers, ester, or prodrug thereof.

In some embodiments, the naphthalene-3-carboxamide is a compound of Formula XI wherein
R⁹² and R⁹³ are hydrogen;
R⁹⁴ is hydrogen or methyl;
R⁹⁵ is selected from the group consisting of hydrogen, hydroxy, halo, substituted alkyl, alkoxy, aryl, substituted aryl, aryloxy and substituted aryloxy;
R⁹⁶ and R⁹⁷ are independently selected from group consisting of hydrogen, alkyl, arylalkylene, and substituted arylalkylene; and
R¹⁰² is —OR¹⁰⁵; wherein R¹⁰⁵ is hydrogen, a cation or alkyl.

The terms “hydroxy” or “hydroxyl” refer to the group —OH.

The term “halo” or “halogen” refers to fluoro, chloro, bromo, and iodo.

The term “cyano” refers to the group —CN.

The term “nitro” refers to the group —NO₂.

The term “carboxyl” refers to —COOH or salts thereof.

The term “alkyl” refers to saturated monovalent hydrocarbyl groups having from 1 to 10 carbon atoms; more particularly, from 1 to 5 carbon atoms; and, even more particularly, 1 to 3 carbon atoms. This term is exemplified by groups such as methyl, ethyl, n-propyl, iso-propyl, n-butyl, t-butyl, n-pentyl, and the like.

The term “cycloalkyl” refers to a saturated or an unsaturated, but nonaromatic, cyclic alkyl groups of from 3 to 10, 3 to 8, or 3 to 6 carbon atoms having single or multiple cyclic rings including, by way of example, adamantyl, cyclopropyl, cyclobutyl, cyclopentyl, cyclooctyl, cyclohexenyl, and the like.

The term “cycloalkoxy” refers to an —O-cycloalkyl group.

The term “aryl” refers to a monovalent aromatic carbocyclic group of from 6 to 14 carbon atoms having a single ring (e.g., phenyl) or multiple condensed rings (e.g., naphthyl or anthryl), which condensed rings may or may not be aromatic (e.g., 2-benzoxazolinone, 2H-1,4-benzoxazin-3(4H)-one-7-yl, and the like) provided that the point of attachment is the aryl group. Preferred aryls include phenyl and naphthyl.

The terms “heterocyclic” or “heterocyclyl” refer to a saturated or unsaturated ring system having a single ring or multiple condensed rings, from 1 to 10 carbon atoms, and from 1 to 4 hetero atoms selected from the group consisting of nitrogen, sulfur, or oxygen within the ring.

The term “heteroaryl” refers to an aromatic heterocyclic group of from 1 to 15 carbon atoms, preferably from 1 to 10 carbon atoms, and 1 to 4 heteroatoms within the ring selected from the group consisting of oxygen, nitrogen, and sulfur. Such heteroaryl groups can have a single ring (e.g., pyridinyl, furyl, or thienyl) or multiple condensed rings (e.g., indolizinyl or benzothienyl), which condensed rings may or may not be aromatic provided the point of attachment is through a ring containing the heteroatom and that ring is aromatic. The nitrogen can optionally be oxidized to provide for the N-oxide, and/or the sulfur ring atoms can optionally be oxidized to provide for the sulfoxide and sulfone derivatives.

Examples of heterocycles and heteroaryls include, but are not limited to, azetidine, pyrrole, imidazole, pyrazole, pyridine, pyrazine, furan, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), piperidinyl, pyrrolidine, tetrahydrofuranyl, and the like.

The term “alkenyl” refers to a vinyl unsaturated monovalent hydrocarbyl group having from 2 to 6, preferably from 2 to 4, carbon atoms, and having at least 1, preferably from 1 to 2, sites of vinyl (>C═C<) unsaturation. Such groups are exemplified by vinyl (ethen-1-yl), allyl, but-3-enyl, and the like.

The term “alkynyl” refers to acetylinic unsaturated monovalent hydrocarbyl groups having from 2 to 6, preferably from 2 to 3, carbon atoms and having at least 1, preferably from 1 to 2, sites of acetylenic (—C≡C—) unsaturation. This group is exemplified by ethyn-1-yl, propyn-1-yl, propyn-2-yl, and the like.

The term “alkoxy” refers to the group “alkyl-O—,” which includes, by way of example, methoxy, ethoxy, n-propoxy, iso-propoxy, n-butoxy, t-butoxy, sec-butoxy, n-pentoxy, and the like.

The term “alkenyloxy” refers to the group “alkenyl-O—.”

The term “alkynyloxy” refers to the group “alkynyl-O—.”

The term “aryloxy” refers to the group aryl-O— that includes, by way of example, phenoxy, naphthoxy, and the like.

The term “aralkyloxy” refers to the group aralkyl-O— that includes, by way of example, benzyloxy, and the like.

The term “carbonyl” refers to C═O.

The term “carbonyloxy” refers to —C(═O)O—.

The terms “aminoacyl” or “amide”, or the prefixes “carbamoyl” or “carboxamide,” refer to the group —C(O)NR^{q}R^{q} where each R^{q} is independently selected from the group consisting of hydrogen, alkyl, alkenyl, alkynyl, aryl, cycloalkyl, heteroaryl, and heterocyclic; or where each R^{q} is joined to form together with the nitrogen atom a heterocyclic wherein alkyl, alkenyl, alkynyl, cycloalkyl, aryl, heteroaryl, and heterocyclic are as defined herein.

The term “amino” refers to the group —NH₂.

The terms “thio” or “mercapto” refer to the group —SH.

The terms “alkylsulfanyl,” “alkylthio,” or “thioether” refer to the groups —S-alkyl where alkyl is as defined above.

The term “sulfinyl” refers to the group —S(O)—.

The term “sulfonyl” refers to the group —S(O)₂—.

The term “heterocyclyloxy” refers to the group —O-heterocyclic.

The term “cycloalkylene” refers to divalent cycloalkyl groups as defined above. The terms “cycloalkylthio” or “cycloalkylsulfanyl” refer to the groups —S-cycloalkyl where cycloalkyl is as defined herein.

The terms “arylthio” or “arylsulfanyl” refer to the group —S-aryl, where aryl is as defined herein.

The terms “heteroarylthio” or “heteroarylsulfanyl” refer to the group —S-heteroaryl, where heteroaryl is as defined herein.

The terms “heterocyclicthio” or “heterocyclicsulfanyl” refer to the group —S-heterocyclic, where heterocyclic is as defined herein.

The term “alkyl alcohol” refers to the group “alkyl-OH”. “Alkyl alcohol” is meant to include methanol, ethanol, 2-propanol, 2-butanol, butanol, etc.

The term “acyl” refers to the groups H—C(O)—, alkyl-C(O)—, alkenyl-C(O)—, alkynyl-C(O)—, cycloalkyl-C(O)—, aryl-C(O)—, heteroaryl-C(O)—, and heterocyclic-C(O)—, provided that a nitrogen atom of the heterocyclic is not bound to the —C(O)— group, wherein alkyl, alkenyl, alkynyl, cycloalkyl, aryl, heteroaryl, and heterocyclic are as defined herein.

The term “acyloxy” refers to the groups alkyl-C(O)O—, alkenyl-C(O)O—, alkynyl-C(O)O—, aryl-C(O)O—, cycloalkyl-C(O)O—, heteroaryl-C(O)O—, and heterocyclic-C(O)O—, wherein alkyl, alkenyl, alkynyl, cycloalkyl, aryl, heteroaryl, and heterocyclic are as defined herein.

The term “alkenyl” refers to a vinyl unsaturated monovalent hydrocarbyl group having from 2 to 6 carbon atoms, and preferably 2 to 4 carbon atoms, and having at least 1, and preferably from 1 to 2 sites of vinyl (>C═C<) unsaturation. Such groups are exemplified by vinyl (ethen-1-yl), allyl, but-3-enyl and the like.

The term “alkynyl” refers to acetylinic unsaturated monovalent hydrocarbyl groups having from 2 to 6, preferably from 2 to 3, carbon atoms and having at least 1, preferably from 1 to 2, sites of acetylenic (—C≡C—) unsaturation. This group is exemplified by ethyn-1-yl, propyn-1-yl, propyn-2-yl, and the like.

The term “acylamino” refers to the groups —NR^{t}C(O)alkyl, —NR^{t}C(O)cycloalkyl, —NR^{t}C(O)alkenyl, —NR^{t}C(O)alkynyl, —NR^{t}C(O)aryl, —NR^{t}C(O)heteroaryl, and —NR^{t}C(O)heterocyclic where R^{t} is hydrogen or alkyl, and wherein alkyl, alkenyl, alkynyl, cycloalkyl, aryl, heteroaryl, and heterocyclic are defined herein.

The term “carbonyloxyamino” refers to the groups —NR^{u}C(O)O-alkyl, —NR^{u}C(O)O-alkenyl, —NR^{u}C(O)O-alkynyl, —NR^{u}C(O)O-cycloalkyl, —NR^{u}C(O)O-aryl, —NR^{u}C(O)O-heteroaryl, and —NR^{u}C(O)O-heterocyclic, where R^{u} is hydrogen or alkyl and wherein alkyl, alkenyl, alkynyl, cycloalkyl, aryl, heteroaryl, and heterocyclic are as defined herein.

The term “oxycarbonylamino” refers to the groups —NR^{u}C(O)O-alkyl, —NR^{u}C(O)O-alkenyl, —NR^{u}C(O)O-alkynyl, —NR^{u}C(O)O-cycloalkyl, —NR^{u}C(O)O-aryl, —NR^{u}C(O)O-heteroaryl, and —NR^{u}C(O)O-heterocyclic, where R^{u} is hydrogen or alkyl, and wherein alkyl, alkenyl, alkynyl, cycloalkyl, aryl, heteroaryl, and heterocyclic are as defined herein.

The term “oxythiocarbonylamino” refers to the groups —NR^{u}C(S)O-alkyl, —NR^{u}C(S)O-alkenyl, —NR^{u}C(S)O-alkynyl, —NR^{u}C(S)O-cycloalkyl, —NR^{u}C(S)O-aryl, —NR^{u}C(S)O-heteroaryl, and —NR^{u}C(S)O-heterocyclic, where R^{u} is hydrogen or alkyl, and wherein alkyl, alkenyl, alkynyl, cycloalkyl, aryl, heteroaryl, and heterocyclic are as defined herein.

The term “aminocarbonyloxy” or the prefix “carbamoyloxy” refer to the groups —OC(O)NR^{v}R^{v} where each R^{v} is independently selected from the group consisting of hydrogen, alkyl, alkenyl, alkynyl, cycloalkyl, aryl, heteroaryl, and heterocyclic; or where each R^{v} is joined to form, together with the nitrogen atom, a heterocyclic, and wherein alkyl, alkenyl, alkynyl, cycloalkyl, aryl, heteroaryl, substituted heteroaryl, and heterocyclic are as defined herein.

The term “aminocarbonylamino” refers to the group —NR^{w}C(O)N(R^{w})₂ where each R^{w} is independently selected from the group consisting of hydrogen and alkyl.

The term “aminothiocarbonylamino” refers to the group —NR^{w}C(S)N(R^{w})₂ where each R^{w} is independently selected from the group consisting of hydrogen and alkyl.

The term “aryloxyaryl” refers to the group -aryl-O-aryl.

The term “carboxyl ester” refers to the groups —C(O)O-alkyl, —C(O)O-alkenyl, —C(O)O-alkynyl, —C(O)O-cycloalkyl, —C(O)O-aryl, —C(O)O-substituted aryl, —C(O)O-heteroaryl, —C(O)O-substituted heteroaryl, —C(O)O-heterocyclic, and —C(O)O-substituted heterocyclic.

The term “cycloalkylene” refers to divalent cycloalkyl groups as defined above.

The term “heteroaryloxy” refers to the group —O-heteroaryl.

The term “sulfonyl” refers to the group —S(O)₂—, and may be included in the groups —S(O)₂H, —SO₂-alkyl, —SO₂-alkenyl, —SO₂-alkynyl, —SO₂-cycloalkyl, —SO₂-cycloalkenyl, —SO₂-aryl, —SO₂-substituted aryl, —SO₂-heteroaryl, and —SO₂-heterocyclic, wherein alkyl, alkenyl, alkynyl, cycloalkyl, cycloalkenyl, aryl, heteroaryl, and heterocyclic are as defined herein.

The term “heterocyclyloxy” refers to the group —O-heterocyclic.

The terms “arylthio” or “arylsulfanyl” refer to the group —S-aryl.

The terms “heteroarylthio” or “heteroarylsulfanyl” refer to the group —S-heteroaryl.

The terms “heterocyclicthio” or “heterocyclicsulfanyl” refer to the group —S-heterocyclic.

Conjugated terms refer to a linear arrangement of the separate substituents as each separate term is defined herein. For example, the term “aralkyl” refers to an aryl-alkyl group and includes, by way of example, benzyl; the term “aralkylcarbamoyl” refers to an aryl-alkyl-carbomoyl substituent wherein each term is as defined herein, etc.

It is understood that in all substituted and conjugated groups as defined herein, polymers arrived at by defining substituents with further substituents to themselves (e.g., aryl having a substituted aryl group as a substituent which is itself substituted with a substituted aryl group, etc.) are not intended for inclusion herein. Also not included are infinite numbers of substituents, whether the substituents are the same or different. In such cases, the maximum number of such substituents is three.

Similarly, it is understood that the above definitions are not intended to include impermissible substitution patterns (e.g., methyl substituted with 5 fluoro groups or a hydroxyl group alpha to ethenylic or acetylenic unsaturation). Such impermissible substitution patterns are well known to the skilled artisan.

The term “pharmaceutically acceptable salt” refers to pharmaceutically acceptable salts of a compound, which salts are derived from a variety of organic and inorganic counter ions well known in the art, and include, by way of example only, sodium, potassium, calcium, magnesium, ammonium, tetraalkylammonium, and the like; and, when the molecule contains a basic functionality, salts of organic or inorganic acids, such as hydrochloride, hydrobromide, tartrate, mesylate, acetate, maleate, oxalate, and the like.

The terms “stereoisomer” or “stereoisomers” refer to compounds that differ in the chirality of one or more stereocenters. Stereoisomers include enantiomers (compounds are non-superimposable mirror images) and diastereomers (compounds having more than one stereogenic center that are non-mirror images of each other and wherein one or more stereogenic center differs between the two stereoisomers). The compounds of the invention can be present as a mixture of stereoisomers or as a single stereoisomer.

The term “tautomer” refers to alternate forms of a compound that differ in the position of a proton, such as enol, keto, and imine enamine tautomers, or the tautomeric forms of heteroaryl groups containing a ring atom attached to both a ring NH moiety and a ring ═N moiety such as pyrazoles, imidazoles, benzimidazoles, triazoles, and tetrazoles.

The term “prodrug,” as used herein, refers to compounds that include chemical groups which, in vivo, can be converted into the carboxylate group and/or can be split off from the amide N-atom and/or can be split off from the R atom to provide for the active drug, a pharmaceutically acceptable salt thereof, or a biologically active metabolite thereof. Suitable groups are well known in the art and particularly include: for the carboxylic acid moiety, a prodrug selected from, e.g., esters including, but not limited to, those derived from alkyl alcohols, substituted alkyl alcohols, hydroxy substituted aryls and heteroaryls and the like; amides, particularly amides derived from amines of the Formula HNR²⁰⁰R²¹⁰ where R²¹⁰ independently hydrogen, alkyl, substituted alkyl, aryl, substituted aryl, and the like; hydroxymethyl, aldehyde and derivatives thereof. The term “ester” refers to compounds that include the group —COOR where R is alkyl, substituted alkyl, alkoxy, or substituted alkoxy.

### Pharmaceutical Formulations and Routes of Administration

The compositions of the present invention can be delivered directly or in pharmaceutical compositions containing excipients, as is well known in the art. The present methods of treatment involve administration of an effective amount of a compound of the present invention to a subject in need, wherein the subject has MS.

An effective amount, e.g., dose, of compound or drug can readily be determined by routine experimentation, as can an effective and convenient route of administration and an appropriate formulation. Various formulations and drug delivery systems are available in the art. (See, e.g., Gennaro, ed. (2000) Remington's Pharmaceutical Sciences, supra; and Hardman, Limbird, and Gilman, eds. (2001) The Pharmacological Basis of Therapeutics, supra.)

Suitable routes of administration may, for example, include oral, rectal, topical, nasal, pulmonary, ocular, intestinal, and parenteral administration. Primary routes for parenteral administration include intravenous, intramuscular, and subcutaneous administration. Secondary routes of administration include intraperitoneal, intra-arterial, intra-articular, intracardiac, intracisternal, intradermal, intralesional, intraocular, intrapleural, intrathecal, intrauterine, and intraventricular administration. The indication to be treated, along with the physical, chemical, and biological properties of the drug, dictate the type of formulation and the route of administration to be used, as well as whether local or systemic delivery would be preferred.

In specific embodiments, the compounds of the present invention are administered orally. For example, in certain embodiments, the invention provides for oral administration of [(1-Cyano-4-hydroxy-5-phenoxy-isoquinoline-3-carbonyl)-amino]-acetic acid (Compound A), [(1-Cyano-4-hydroxy-5-p-tolyloxy-isoquinoline-3-carbonyl)-amino]-acetic acid (Compound B), [(4-Hydroxy-1-pyridin-3-yl-8-p-tolyloxy-isoquinoline-3-carbonyl)-amino]-acetic acid (Compound C), {[7-(3-Fluoro-5-methoxy-phenoxy)-4-hydroxy-isoquinoline-3-carbonyl]amino}-acetic acid (Compound D), {[4-Hydroxy-8-(3-methoxy-phenoxy)-isoquinoline-3-carbonyl]-amino}-acetic acid (Compound E), [(4-Hydroxy-7-phenoxy-isoquinoline-3-carbonyl)-amino]-acetic acid (Compound F), {[4-Hydroxy-2-oxo-1-(4-trifluoromethyl-benzyl)-1,2-dihydro-pyrrolo[1,2-b]pyridazine-3-carbonyl]-amino}-acetic acid (Compound G), (S)-2-{[6-Chloro-4-hydroxy-2-oxo-1-(4-trifluoromethyl-benzyl)-1,2-dihydro-pyrrolo[1,2-b]pyridazine-3-carbonyl]-amino}-propionic acid (Compound H), {[6-Chloro-1-(4-chloro-benzyl)-4-hydroxy-2-oxo-1,2-dihydro-pyrrolo[1,2-b]pyridazine-3-carbonyl]-amino}-acetic acid (Compound I), {[4-Hydroxy-2-oxo-7-(4-phenoxy-phenyl)-2H-chromene-3-carbonyl]-amino}-acetic acid (Compound J), [(6-Hexyloxy-4-hydroxy-2-oxo-2H-chromene-3-carbonyl)-amino]-acetic acid (Compound K), {[4-Hydroxy-7-(4-methoxy-phenyl)-2-oxo-2H-thiochromene-3-carbonyl]-amino}-acetic acid (Compound L), [(7-Butoxy-4-hydroxy-2-oxo-2H-thiochromene-3-carbonyl)-amino]-acetic acid (Compound M), [(7-Chloro-1-hydroxy-4,4-dimethyl-3-oxo-3,4-dihydro-naphthalene-2-carbonyl)-amino]-acetic acid (Compound N), {[7-Cyano-1-(2-fluoro-benzyl)-4-hydroxy-1H-pyrrolo[2,3-c]pyridine-5-carbonyl]-amino}-acetic acid (Compound O), [(1-Biphenyl-4-ylmethyl-7-cyano-4-hydroxy-1H-pyrrolo[2,3-c]pyridine-5-carbonyl)-amino]-acetic acid (Compound P), or {[2,3-Dichloro-7-cyano-4-hydroxy-1-(4-methoxy-benzyl)-1H-pyrrolo[2,3-c]pyridine-5-carbonyl]-amino}-acetic acid (Compound Q).

Pharmaceutical dosage forms of a compound of the invention may be provided in an instant release, controlled release, sustained release, or target drug-delivery system. Commonly used dosage forms include, for example, solutions and suspensions, (micro-) emulsions, ointments, gels and patches, liposomes, tablets, dragees, soft or hard shell capsules, suppositories, ovules, implants, amorphous or crystalline powders, aerosols, and lyophilized formulations. Depending on route of administration used, special devices may be required for application or administration of the drug, such as, for example, syringes and needles, inhalers, pumps, injection pens, applicators, or special flasks. Pharmaceutical dosage forms are often composed of the drug, an excipient(s), and a container/closure system. One or multiple excipients, also referred to as inactive ingredients, can be added to a compound of the invention to improve or facilitate manufacturing, stability, administration, and safety of the drug, and can provide a means to achieve a desired drug release profile. Therefore, the type of excipient(s) to be added to the drug can depend on various factors, such as, for example, the physical and chemical properties of the drug, the route of administration, and the manufacturing procedure. Pharmaceutically acceptable excipients are available in the art, and include those listed in various pharmacopoeias. (See, e.g., USP, JP, EP, and BP, FDA web page (www.fda.gov), Inactive Ingredient Guide 1996, and Handbook of Pharmaceutical Additives, ed. Ash; Synapse Information Resources, Inc. 2002.)

Pharmaceutical dosage forms of a compound of the present invention may be manufactured by any of the methods well-known in the art, such as, for example, by conventional mixing, sieving, dissolving, melting, granulating, dragee-making, tabletting, suspending, extruding, spray-drying, levigating, emulsifying, (nano/micro-) encapsulating, entrapping, or lyophilization processes. As noted above, the compositions of the present invention can include one or more physiologically acceptable inactive ingredients that facilitate processing of active molecules into preparations for pharmaceutical use.

Proper formulation is dependent upon the desired route of administration. For intravenous injection, for example, the composition may be formulated in aqueous solution, if necessary using physiologically compatible buffers, including, for example, phosphate, histidine, or citrate for adjustment of the formulation pH, and a tonicity agent, such as, for example, sodium chloride or dextrose. For transmucosal or nasal administration, semisolid, liquid formulations, or patches may be preferred, possibly containing penetration enhancers. Such penetrants are generally known in the art. For oral administration, the compounds can be formulated in liquid or solid dosage forms and as instant or controlled/sustained release formulations. Suitable dosage forms for oral ingestion by a subject include tablets, pills, dragees, hard and soft shell capsules, liquids, gels, syrups, slurries, suspensions, and emulsions. The compounds may also be formulated in rectal compositions, such as suppositories or retention enemas, e.g., containing conventional suppository bases such as cocoa butter or other glycerides.

Solid oral dosage forms can be obtained using excipients, which may include, fillers, disintegrants, binders (dry and wet), dissolution retardants, lubricants, glidants, antiadherants, cationic exchange resins, wetting agents, antioxidants, preservatives, coloring, and flavoring agents. These excipients can be of synthetic or natural source. Examples of such excipients include cellulose derivatives, citric acid, dicalcium phosphate, gelatine, magnesium carbonate, magnesium/sodium lauryl sulfate, mannitol, polyethylene glycol, polyvinyl pyrrolidone, silicates, silicium dioxide, sodium benzoate, sorbitol, starches, stearic acid or a salt thereof, sugars (i.e. dextrose, sucrose, lactose, etc.), talc, tragacanth mucilage, vegetable oils (hydrogenated), and waxes. Ethanol and water may serve as granulation aides. In certain instances, coating of tablets with, for example, a taste-masking film, a stomach acid resistant film, or a release-retarding film is desirable. Natural and synthetic polymers, in combination with colorants, sugars, and organic solvents or water, are often used to coat tablets, resulting in dragees. When a capsule is preferred over a tablet, the drug powder, suspension, or solution thereof can be delivered in a compatible hard or soft shell capsule.

In one embodiment, the compounds of the present invention can be administered topically, such as through a skin patch, a semi-solid or a liquid formulation, for example a gel, a (micro)-emulsion, an ointment, a solution, a (nano/micro)-suspension, or a foam. The penetration of the drug into the skin and underlying tissues can be regulated, for example, using penetration enhancers; the appropriate choice and combination of lipophilic, hydrophilic, and amphiphilic excipients, including water, organic solvents, waxes, oils, synthetic and natural polymers, surfactants, emulsifiers; by pH adjustment; and use of complexing agents. Other techniques, such as iontophoresis, may be used to regulate skin penetration of a compound of the invention. Transdermal or topical administration would be preferred, for example, in situations in which local delivery with minimal systemic exposure is desired.

For administration by inhalation, or administration to the nose, the compounds for use according to the present invention are conveniently delivered in the form of a solution, suspension, emulsion, or semisolid aerosol from pressurized packs, or a nebuliser, usually with the use of a propellant, e.g., halogenated carbons derived from methane and ethane, carbon dioxide, or any other suitable gas. For topical aerosols, hydrocarbons like butane, isobutene, and pentane are useful. In the case of a pressurized aerosol, the appropriate dosage unit may be determined by providing a valve to deliver a metered amount. Capsules and cartridges of, for example, gelatin, for use in an inhaler or insufflator, may be formulated. These typically contain a powder mix of the compound and a suitable powder base such as lactose or starch.

Compositions formulated for parenteral administration by injection are usually sterile and, can be presented in unit dosage forms, e.g., in ampoules, syringes, injection pens, or in multi-dose containers, the latter usually containing a preservative. The compositions may take such forms as suspensions, solutions, or emulsions in oily or aqueous vehicles, and may contain formulatory agents, such as buffers, tonicity agents, viscosity enhancing agents, surfactants, suspending and dispersing agents, antioxidants, biocompatible polymers, chelating agents, and preservatives. Depending on the injection site, the vehicle may contain water, a synthetic or vegetable oil, and/or organic co-solvents. In certain instances, such as with a lyophilized product or a concentrate, the parenteral formulation would be reconstituted or diluted prior to administration. Depot formulations, providing controlled or sustained release of a compound of the invention, may include injectable suspensions of nano/micro particles or nano/micro or non-micronized crystals. Polymers such as poly(lactic acid), poly(glycolic acid), or copolymers thereof, can serve as controlled/sustained release matrices, in addition to others well known in the art. Other depot delivery systems may be presented in form of implants and pumps requiring incision.

Suitable carriers for intravenous injection for the molecules of the invention are well-known in the art and include water-based solutions containing a base, such as, for example, sodium hydroxide, to form an ionized compound, sucrose or sodium chloride as a tonicity agent, for example, the buffer contains phosphate or histidine. Co-solvents, such as, for example, polyethylene glycols, may be added. These water-based systems are effective at dissolving compounds of the invention and produce low toxicity upon systemic administration. The proportions of the components of a solution system may be varied considerably, without destroying solubility and toxicity characteristics. Furthermore, the identity of the components may be varied. For example, low-toxicity surfactants, such as polysorbates or poloxamers, may be used, as can polyethylene glycol or other co-solvents, biocompatible polymers such as polyvinyl pyrrolidone may be added, and other sugars and polyols may substitute for dextrose.

For composition useful for the present methods of treatment, a therapeutically effective dose can be estimated initially using a variety of techniques well-known in the art. Initial doses used in animal studies may be based on effective concentrations established in cell culture assays. Dosage ranges appropriate for human subjects can be determined, for example, using data obtained from animal studies and cell culture assays.

A therapeutically effective dose or amount of a compound, agent, or drug of the present invention refers to an amount or dose of the compound, agent, or drug that results in amelioration of symptoms or a prolongation of survival in a subject. Toxicity and therapeutic efficacy of such molecules can be determined by standard pharmaceutical procedures in cell cultures or experimental animals, e.g., by determining the LD50 (the dose lethal to 50% of the population) and the ED50 (the dose therapeutically effective in 50% of the population). The dose ratio of toxic to therapeutic effects is the therapeutic index, which can be expressed as the ratio LD50/ED50. Agents that exhibit high therapeutic indices are preferred.

The effective amount or therapeutically effective amount is the amount of the compound or pharmaceutical composition that will elicit the biological or medical response of a tissue, system, animal, or human that is being sought by the researcher, veterinarian, medical doctor, or other clinician, e.g., treatment of cancer, including induction of anti-tumor effects, etc.

Dosages preferably fall within a range of circulating concentrations that includes the ED50 with little or no toxicity. Dosages may vary within this range depending upon the dosage form employed and/or the route of administration utilized. The exact formulation, route of administration, dosage, and dosage interval should be chosen according to methods known in the art, in view of the specifics of a subject's condition.

Dosage amount and interval may be adjusted individually to provide plasma levels of the active moiety that are sufficient to achieve the desired effects, i.e., minimal effective concentration (MEC). The MEC will vary for each compound but can be estimated from, for example, in vitro data and animal experiments. Dosages necessary to achieve the MEC will depend on individual characteristics and route of administration. In cases of local administration or selective uptake, the effective local concentration of the drug may not be related to plasma concentration.

In some embodiments of the present invention, effective doses for compounds of the invention include doses of 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, and 30 mg/kg, respectively.

In additional embodiments, effective treatment regimes for compounds of the invention include administration two or three times weekly.

The amount of agent or composition administered may be dependent on a variety of factors, including the sex, age, and weight of the subject being treated, the severity of the affliction, the manner of administration, and the judgment of the prescribing physician.

The present compositions may, if desired, be presented in a pack or dispenser device containing one or more unit dosage forms containing the active ingredient. Such a pack or device may, for example, comprise metal or plastic foil, such as a blister pack, or glass and rubber stoppers such as in vials. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

These and other embodiments of the present invention will readily occur to those of ordinary skill in the art in view of the disclosure herein.

### EXAMPLES

The invention is further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description and accompanying figures. Such modifications fall within the scope of the appended claims.

### Example 1

### Compounds and Methods of the Invention Reduce Disease Severity in EAE Animal Model of Multiple Sclerosis

Experimental allergic encephalomyelitis (EAE, also referred to as experimental autoimmune encephalomyelitis) is currently the best animal model of human multiple sclerosis (MS). In this model, induction of EAE occurs following, for example, injection of spinal cord homogenate or central nervous system (CNS) antigens, such as myelin basic protein (MBP), myelin olidgodendrocyte glycoprotein, or proteolipid protein. Approximately 10 days following injection of CNS antigens, susceptible rats (e.g., Lewis rats) develop a progressive paralysis similar to that observed in humans.

Female Lewis rats of approximately 220 grams were used in this study (Janvier; Le Genest St Isle, France). Compounds of the invention were formulated in water containing 10% cremophor and 60 mM L-histidine to achieve a final concentration of either 2 mg/ml or 6 mg/ml. Dexamethasone was dissolved in saline at a concentration of 0.1 mg/ml.

Induction of EAE by injection with myelin basic protein (MBP) antigen was performed as follows. Prior to injection with the MBP antigen (day 0), rats were anaesthetized by injection of 60 mg/kg of ketamine (Imalgene500®, Rhone Mérieux, Lyon, France) plus 4 mg/kg of xylazine (Rompum 2%, Bayer Pharma, Kiel, Germany). Rats were then injected in the hind footpad with a total volume of 100 μl of MBP inoculum solution per paw containing 100 μg of MBP antigen (Sigma, L'Isle d'Abeau Chesnes, France) emulsified (1:1) containing incomplete Freund's adjuvant (IFA) (Sigma, L'Isle d'Abeau Chesnes, France) and 500 μg of heat-inactivated mycobacterium tuberculosis (strain H 37 RA from Difco). Control rats were similarly injected with an equivalent volume of inoculum without MBP.

Compound of the invention (10 mg/kg or 30 mg/kg) or vehicle control was administered p.o. daily from day 3 to day 21 following MBP injection. Dexamethasone (1 mg/kg) was administered subcutaneously at 1 mg/kg from day 8 to day 12 following MBP injection. Table 1 below shows the treatment schedule used for this experiment.

TABLE 1

| | | Concentration | Treatment |
|---|---|---|---|
| Group Description/Name | Dose Level | (mg/ml) | Days |
| Control/vehicle (p.o.) | 0 mg/kg/d | 0 | 3-21 |
| EAE/vehicle (p.o.) | 0 mg/kg/d | 0 | 3-21 |
| EAE/Cmpd A (10 mg/kg; p.o.) | 10 mg/kg/d | 2 | 3-21 |
| EAE/Cmpd A (30 mg/kg; p.o.) | 30 mg/kg/d | 6 | 3-21 |
| EAE/Dexamethasone | 1 mg/kg/d | 0.1 | 8-12 |
| (1 mg/kg; s.c.) | | | |

Neurological scoring was performed daily from day 7 to day 21 following BP injection. Grading of disease severity (i.e., disease score) was based on the following observations performed by two different observers: 0=no abnormality; 1=distal weakness of the tail; 2=complete weakness of the tail; 3=mild weakness in one or two hindlimbs; 4=moderate paraparesia of one or two hindlimbs; 5=total paraplegia. Analysis of variance (ANOVA) was performed on data from the measured (clinical score) or the calculated (severity score) parameters. Fisher's Protected Least Significant Difference was used for pairwise comparisons. p value≦0.05 were considered significant.

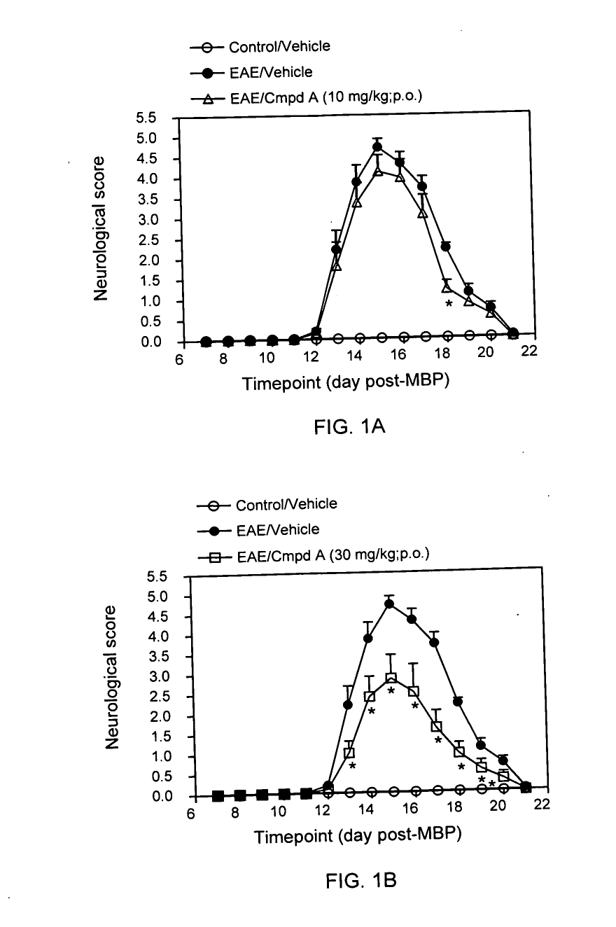

As shown in FIGS. 1A, 1B, and 1C, symptoms of EAE were first observed at approximately day 12 post MBP injection, and peak disease activity occurred from day 14 to day 16, followed by progressive remission. Dexamethasone treatment, used as a positive control treatment in this study, prevented the development of EAE disease. (See FIG. 1C.)

Administration of Compound A resulted in reduced disease severity compared to that observed in vehicle-treated control EAE animals. In particular, animals administered Compound A at 10 mg/kg or 30 mg/kg showed a 17% and 46% (p<0.05) reduction in disease severity, respectively, compared to that observed in vehicle-treated control EAE animals. (See FIGS. 1A and 1B.) These results showed that Compound A reduced clinical severity of disease in an EAE animal model of MS.

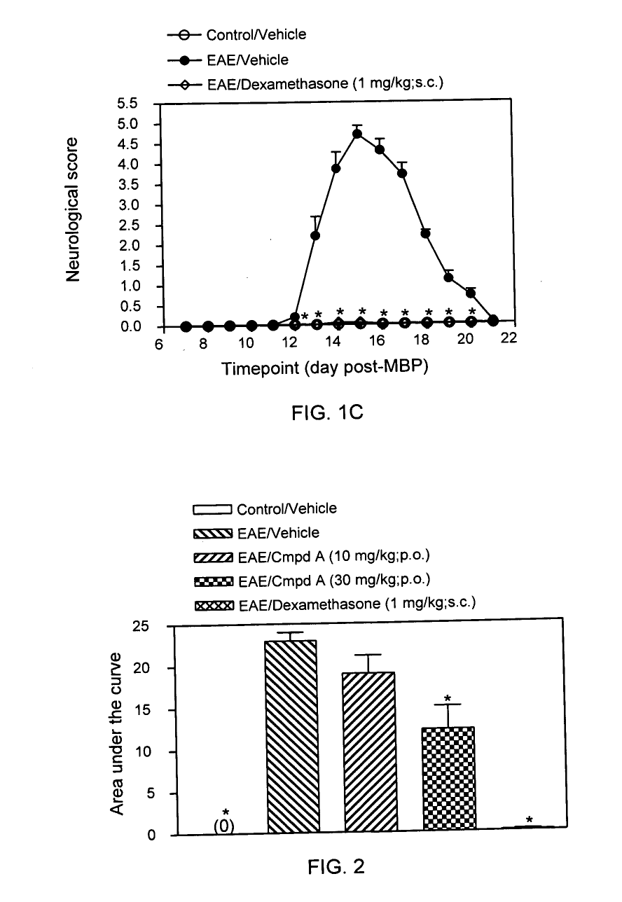

Cumulative EAE disease score, used as a measure of overall disease severity, was determined by adding all disease/neurological scores for each animal over the time course of the experiment. As shown in FIG. 2, cumulative disease score (presented as area under the curve (AUC) in FIG. 2) was reduced in EAE animals administered Compound A at either 10 mg/kg or 30 mg/kg. Taken together, these results showed that administration of Compound A in a rat EAE model of MS resulted in a reduction of clinical disease severity. Collectively, these results indicated that methods and compounds of the present invention are useful for treating MS, reducing the severity of MS symptoms, etc.

In another series of experiments, the effectiveness of compounds and methods of the present invention on treating established multiple sclerosis in an EAE model was examined as follows. EAE was induced in female Lewis rats (200-220 g) as described above. Compound of the invention (30 mg/kg) or vehicle control was administered p.o. daily from day 3 to day 21 following MBP injection (prophylactic group) or from the day first symptoms appeared to day 21 (treatment group). Dexamethasone (1 mg/kg) was administered subcutaneously at 1 mg/kg from day 8 to day 12 following MBP injection. Table 2 below shows the treatment schedule used for this experiment.

TABLE 2

| | | Concentration | Treatment |
|---|---|---|---|
| Group Description/Name | Dose Level | (mg/ml) | Days |
| Control/vehicle (p.o.) | 0 mg/kg/d | 0 | 3-21 |
| EAE/vehicle (p.o.) | 0 mg/kg/d | 0 | 3-21 |
| EAE/Cmpd A (30 mg/kg; p.o.) | 30 mg/kg/d | 6 | 3-21 |
| EAE/vehicle (p.o.) | 0 mg/kg/d | 0 | first symptom |
| | | | to day 21 |
| EAE/Cmpd A (30 mg/kg; p.o.) | 30 mg/kg/d | 6 | first symptom |
| | | | to day 21 |
| EAE/Dexamethasone | 1 mg/kg/d | 0.1 | 8-12 |
| (1 mg/kg; s.c.) | | | |

Neurological scoring was performed daily from day 7 to day 21 following BP injection. Grading of disease severity (i.e., disease score) was based on the following observations performed by two different observers: 0=no abnormality; 1=distal weakness of the tail; 2=complete weakness of the tail; 3=mild weakness in one or two hindlimbs; 4=moderate paraparesia of one or two hindlimbs; 5=total paraplegia. Analysis of variance (ANOVA) was performed on data from the measured (clinical score) or the calculated (severity score) parameters. Fisher's Protected Least Significant Difference was used for pairwise comparisons. p value≦0.05 were considered significant.

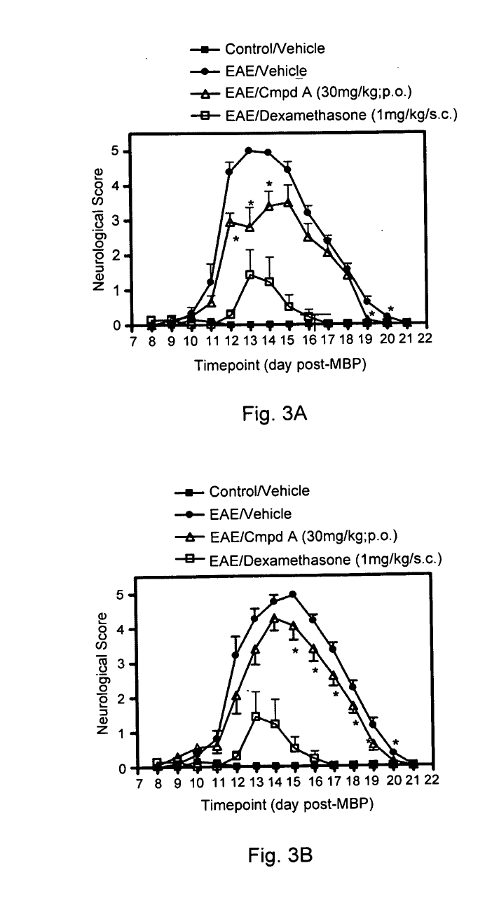

As shown in FIGS. 3A and 3B, symptoms of EAE were first observed at approximately day 11 post MBP injection, and peak disease activity occurred from day 13 to day 15, followed by progressive remission. Dexamethasone treatment, used as a positive control treatment in this study, significantly decreased the development of EAE disease. (See FIGS. 3A and 3B.)

Administration of Compound A from day 3 to day 21 resulted in reduced disease severity in the prophylactic group (FIG. 3A) compared to that observed in vehicle-treated control EAE animals (See FIG. 3A). Similarly, administration of Compound A from the first day symptoms appeared to day 21 resulted in reduced disease severity in the treatment group (FIG. 3B) compared to that observed in vehicle-treated control EAE animals (See FIG. 3B). In particular, prophylactic and treatment animals administered Compound A showed a 31% and 19% (p<0.05) reduction in disease severity, respectively, compared to that observed in vehicle-treated control EAE animals. (See FIGS. 3A and 3B.) These results showed that Compound A reduced clinical severity of disease in an EAE animal model of MS. These results further showed that compounds and methods of the present invention are useful for preventing or treating multiple sclerosis (e.g., reducing the onset of MS or decreasing disease severity of MS).

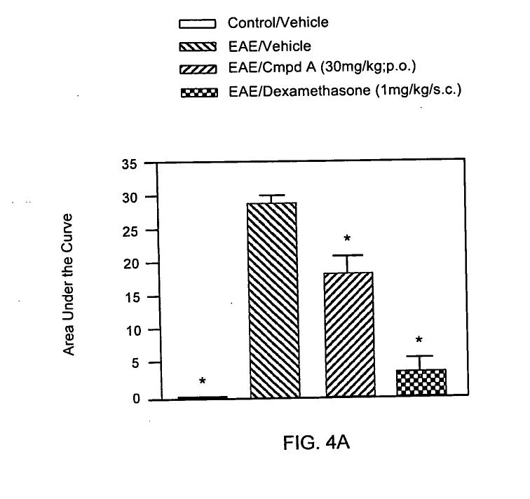

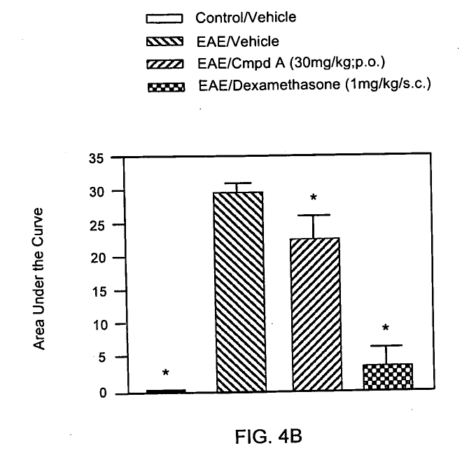

Cumulative EAE disease score, used as a measure of overall disease severity, was determined by adding all disease/neurological scores for each animal over the time course of the experiment. As shown in FIGS. 4A and 4B, cumulative disease score (presented as area under the curve (AUC) in FIGS. 4A and 4B) was reduced in EAE animals administered Compound A in both the prophylactic group (FIG. 4A) and treatment group (FIG. 4B). Taken together, these results showed that administration of Compound A in a rat EAE model of MS resulted in a reduction of clinical disease severity. Collectively, these results indicated that methods and compounds of the present invention are useful for preventing or treating MS. These results further showed that methods and compounds of the present invention are useful for reducing or ameliorating symptoms of MS (e.g., weakness or diminished dexterity in one or more limbs, muscle weakness, difficulty in moving (e.g., disturbance of gait), difficulties with coordination and balance (ataxia), fatigue, etc.).

### Example 2

### Compounds and Methods of the Invention Reduce Disease Severity in an EAE Animal Model of Chronic Progressive Multiple Sclerosis

A chronic progressive model of EAE is an accepted animal model of human chronic progressive multiple sclerosis (MS). In this model, induction of chronic progressive EAE occurs following injections of myelin oligodendrocyte glycoprotein (MOG). Approximately 8 to 14 days after injection of MOG, animals develop chronic progressive paralysis similar to that observed in humans with chronic progressive MS.

Female C57BL/6 mice (8-10 weeks old) were used in this study (Taconic Farm, CA). Compounds of the present invention were formulated in 0.5% carboxymethyl cellulose (CMC) with 0.1% Polysorbate 80 to achieve a final concentration of either 1.2 mg/ml or 4.0 mg/ml. FTY-720 was also formulated in 0.5% carboxymethyl cellulose (CMC) with 0.1% Polysorbate 80 to achieve a final concentration of 2.0 mg/ml.

Induction of chronic progressive EAE by injection with myelin oligodendrocyte glycoprotein (MOG) was performed as follows. Mice were injected subcutaneously with 200 μg of myelin oligodendrocyte glycoprotein (MOG35-55) peptide emulsion in both sides of their pectoral regions (50 μl each side). At 8 hours and 48 hr after immunization with MOG, animals were injected intraperitoneally with 400 ng of pertussis toxin (PT) in 100 μl of saline. Control mice received both injections with an equivalent volume of saline without MOG or PT.

Compound of the invention (6 mg/kg or 20 mg/kg) or vehicle control was administered p.o. three times a week from day 0 to day 35. FTY-720 (10 mg/kg) was used in this study as a positive control and was administered daily from day 0 to day 35. Table 2 below shows the treatment schedule used for this experiment.

TABLE 3

| Group Description/ | | Dosing | Treatment |
|---|---|---|---|
| Name | Dose Level | Frequency | Days |
| Control/vehicle | 0 mg/kg/d | 3 times a week | 0-35 |
| EAE/vehicle | 0 mg/kg/d | 3 times a week | 0-35 |
| EAE/Cmpd A | 6 mg/kg/d | 3 times a week | 0-35 |
| EAE/Cmpd A | 20 mg/kg/d | 3 times a week | 0-35 |
| EAE/FTY-720 | 10 mg/kg/d | daily | 0-35 |

Neurological scoring was performed daily from day 7 to day 35 following MOG injection. Grading of disease severity (i.e., disease score) was based on the following observations performed by two different observers: 0=no abnormality; 1=distal weakness of the tail (limp tail); 2=limp tail and weakness of hind legs (mild paraparesis); 3=limp tail and complete paralysis of hind legs or paralysis in one of one front leg and one hind leg (moderate paraparesis); 4=limp tail, complete hind leg and partial front leg paralysis (moderate quadraparesis); 5=complete hind and front leg paralysis (moribund). Analysis of variance (ANOVA) was performed on data from the measured (neurological score) parameters. p value≦0.05 were considered significant.

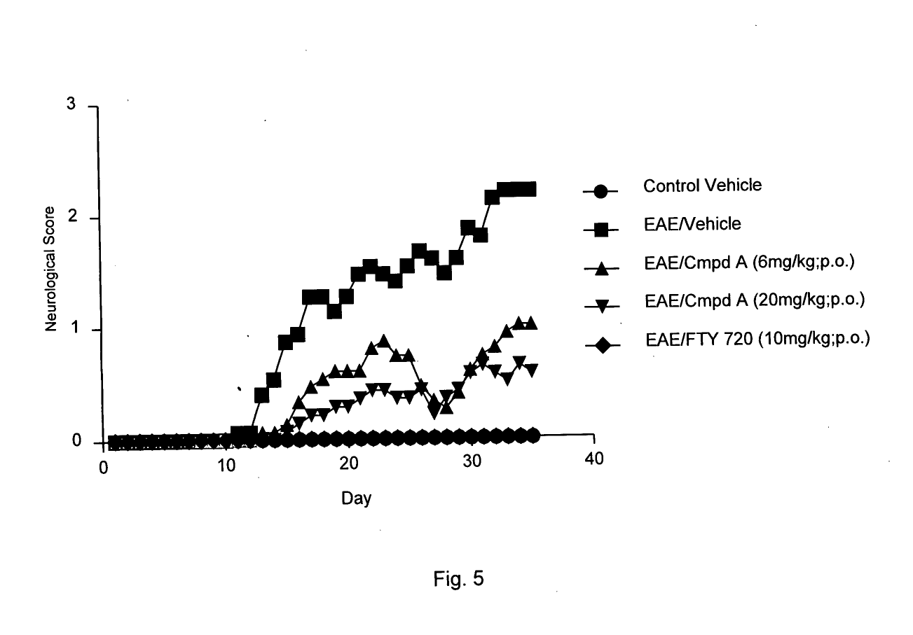

As shown in FIG. 5, symptoms of EAE were first observed at approximately day 11 post MOG injection with disease activity progressing through day 35 in the vehicle-treated control EAE animals. Treatment with FTY-720, used as a positive control treatment in this study, prevented the development of EAE disease. (See FIG. 5.)

Administration of Compound A resulted in reduced disease severity compared to that observed in vehicle-treated control EAE animals. In particular, animals administered Compound A at 10 mg/kg or 30 mg/kg showed a reduction in disease severity at all timepoints compared to that observed in vehicle-treated control EAE animals. (See FIG. 5.) These results showed that Compound A reduced clinical severity of disease in an EAE animal model of chronic progressive MS. These results further showed that methods and compounds of the present invention are useful for reducing or ameliorating symptoms of MS (e.g., weakness or diminished dexterity in one or more limbs, muscle weakness, difficulty in moving (e.g., disturbance of gait), difficulties with coordination and balance (ataxia), fatigue, etc.).

Table 4 below reports the number of animals in each group that showed no disease activity through day 35. Administration of Compound A at 10 mg/kg or 30 mg/kg prevented the development of EAE disease in 40% and 43% (p<0.05) of treated animals, respectively, compared to that observed in vehicle-treated control EAE animals. (See Table 4.)

TABLE 4

| Group Description/Name | No. of Animals Showing No Disease Activity |
|---|---|
| Control/vehicle | 10/10 |
| EAE/vehicle | 1/15 |
| EAE/Cmpd A | 6/15 |
| EAE/Cmpd A | 6/14 |
| EAE/FTY-720 | 7/7 |

Taken together, these results showed that administration of Compound A in a rat EAE model of chronic progressive MS resulted in a reduction of clinical disease severity. Collectively, these results indicated that methods and compounds of the present invention are useful for treating MS (e.g., reducing the severity of MS symptoms, etc.). In particular, these results indicated that methods and compounds of the present invention are useful for treating chronic progressive MS. These results further showed that methods and compounds of the present invention are useful for reducing or ameliorating symptoms of MS in subjects having chronic progressive MS (e.g., weakness or diminished dexterity in one or more limbs, muscle weakness, difficulty in moving (e.g., disturbance of gait), difficulties with coordination and balance (ataxia), fatigue, etc.).

### Example 3

### Compounds and Methods of the Invention Improve Oligodendrocyte Viability in an In-Vitro Model of Multiple Sclerosis

Oligodendrocyte apoptosis has been shown to contribute to the pathology of multiple sclerosis (MS) (See, e.g., Boccaccio and Steinman (1996) J Neurosci Res. 45:647-654; Lucchinetti et al. (1996) Brain Pathol. 6:259-274.) Accordingly, ceramide-induced cell death of oligodendrocytes in culture has been used as an in-vitro model of multiple sclerosis. (See e.g., Craighead et al. (2000) Neurosci Lett. 278:125-8; Jana et al. (2007) J Neuroimmune Pharmacol. 2:184-93; Jana et al. (2009) J Neurol Sci. 278:5-15; Singh et al. (1998) J Biol. Chem. 273:20354-62.) In this model, addition of ceramide to cultured oligodendrocytes results in oxidative stress-induced cell death (i.e. apoptosis). The effect of compounds and methods of the present invention on the ability to improve oligodendrocyte viability following ceramide challenge was examined as follows. Oligodendrocytes (MO3.13 cells; Cedarlane Laboratories, Burlington, N.C.) were plated in 96-well culture dishes at a density of 4000 cells per well and cultured at 37° C., 10% CO2 in DMEM containing L-glutamine, sodium pyruvate, and 25 mM glucose (Mediatech Inc., Manassas, Va.) and supplemented with 10% FBS (Invitrogen Corporation, Carlsbad, Calif.). The next day, the plating media was removed and replaced with DMEM containing L-glutamine (Invitrogen Corporation, Carlsbad, Calif.) and supplemented with 5.5 mM glucose. Cells were then treated with vehicle (0.015% DMSO) or ceramide (Sigma-Aldrich, St. Louis, Mo.) plus vehicle (0.05% DMSO) or a compound of the invention (20 μM).

For cells treated longer than 3 days, half the media was removed after the third day of treatment and replaced with an equal volume of fresh treatment media containing vehicle (0.015% DMSO) or ceramide plus vehicle (0.05% DMSO) or a compound of the invention.

Cell viability was assessed 3, 4, and 5 days after treatment using the following protocol. Treatment media was removed from the cells and 100 ul of 1.67 uM Calcein AM reagent (Invitrogen Corporation, Carlsbad, Calif.) prepared in DMEM containing L-glutamine (Invitrogen Corporation, Carlsbad, Calif.) and supplemented with 5.5 mM glucose was added to each well. Cells were returned to 37° C. in a 10% CO2 incubator for approximately 30 minutes before assessing fluorescence (Ex 485 nM/Em 538 nM) using a microplate reader (Molecular Devices, Sunnyvale, Calif.). Images were taken of representative wells using a fluorescent microscope equipped with a camera (Nikon, Melville, N.Y.) and imaging software (Nikon, Melville, N.Y.).

As shown below in Table 5, addition of ceramide for 3, 4 or 5 days reduced the viability of expanded oligodendrocytes to that observed in non-ceramide-treated control cells. Cells treated with compounds of the present invention showed increased viability compared to that observed in vehicle treated cells. In particular, ceramide-challenged oligodendrocytes treated with Compound A showed a 3-fold increase in viability at day 5 compared to cell viability observed in vehicle treated oligodendrocytes. (See Table 5.)

TABLE 5

| | Cera- | Com- | % Viable | % Viable | % Viable |
|---|---|---|---|---|---|
| | mide | pound | at | at | at |
| Group | (μM) | (μM) | Day 3 | Day 4 | Day 5 |
| Control/vehicle | 0 | 0 | 100 | 100 | 100 |
| Ceramide/vehicle | 2.25 | 20 | 78 | 64 | 11 |
| Ceramide/Cmpd A | 2.25 | 20 | 82 | 46 | 33 |
| Ceramide/Cmpd B | 2.25 | 20 | 76 | 54 | 16 |
| Ceramide/Cmpd F | 2.25 | 20 | 85 | 66 | 62 |
| Control/vehicle | 0 | 0 | 100 | 100 | 100 |
| Ceramide/vehicle | 2.25 | 20 | 80 | 42 | 32 |
| Ceramide/Cmpd C | 2.25 | 20 | 61 | 51 | 16 |
| Ceramide/Cmpd P | 2.25 | 20 | 79 | 80 | 77 |
| Ceramide/Cmpd G | 2.25 | 20 | 94 | 86 | 80 |
| Control/vehicle | 0 | 0 | 100 | 100 | 100 |
| Ceramide/vehicle | 2.75 | 20 | 49 | 16 | 10 |
| Ceramide/Cmpd F | 2.75 | 20 | 72 | 53 | 51 |
| Ceramide/Cmpd D | 2.75 | 20 | 79 | 74 | 61 |
| Ceramide/Cmpd E | 2.75 | 20 | 84 | 74 | 71 |
| Control/vehicle | 0 | 0 | 100 | 100 | 100 |
| Ceramide/vehicle | 2.75 | 20 | 43 | 7 | 6 |
| Ceramide/Cmpd Q | 2.75 | 20 | 70 | 59 | 50 |
| Ceramide/Cmpd O | 2.75 | 20 | 74 | 53 | 52 |
| Ceramide/Cmpd J | 2.75 | 20 | 70 | 36 | 22 |
| Control/vehicle | 0 | 0 | 100 | 100 | 100 |
| Ceramide/vehicle | 2.75 | 20 | 45 | 15 | 28 |
| Ceramide/Cmpd N | 2.75 | 20 | 82 | 67 | 67 |
| Ceramide/Cmpd L | 2.75 | 20 | 75 | 59 | 64 |
| Ceramide/Cmpd M | 2.75 | 20 | 74 | 57 | 59 |
| Control/vehicle | 0 | 0 | 100 | 100 | 100 |
| Ceramide/vehicle | 2.75 | 20 | 57 | 7 | 7 |
| Ceramide/Cmpd H | 2.75 | 20 | 73 | 74 | 77 |
| Ceramide/Cmpd I | 2.75 | 20 | 76 | 66 | 64 |
| Ceramide/Cmpd K | 2.75 | 20 | 72 | 43 | 56 |

Taken together, these results showed that methods and compounds of the present invention improve oligodendrocyte viability following ceramide challenge. In vitro assays of ceramide-induced cell death of oligodendrocytes have been used as a model for multiple sclerosis (MS). Thus, these results suggested that methods and compounds of the present invention would be useful for treating MS and reducing or ameliorating symptoms of MS.

Various modifications of the invention, in addition to those shown and described herein, will become apparent to those skilled in the art from the foregoing description. Such modifications are intended to fall within the scope of the appended claims.

All references cited herein are hereby incorporated by reference herein in their entirety.

What is claimed is:

1. A method for treating multiple sclerosis in a subject, the method comprising administering to the subject a therapeutically effective amount of an agent that inhibits hypoxia inducible factor (HIF) prolyl hydroxylase activity.

2. The method of claim 1, wherein the subject has relapsing-remitting multiple sclerosis.

3. The method of claim 1, wherein the subject has secondary progressive multiple sclerosis.

4. The method of claim 1, wherein the subject has primary progressive multiple sclerosis.

5. The method of claim 1, wherein the subject has progressive relapsing multiple sclerosis.

6. The method of claim 1, wherein the agent is a cyclic carboxamide.

7. The method of claim 6, wherein the cyclic carboxamide is a carbocyclic carboxamide or a heterocyclic carboxamide.

8. The method of claim 1, wherein the method further comprises administering to the subject an additional therapeutic agent.
